SEQUENCE LISTING (44 sequences)

SEQ ID NO: 1 - protein, 725 aa, Homo sapiens
  misc_feature: Incyte ID No 7497502CD1
MGLWWVTVQPPARRMGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKHRGRVRESEMCTEGLLAPVGACEGDYGGPLACFTHNCWVLEGIIIPNRVCARSRWPAVFTRVSVFVDWIHKVMRLG

SEQ ID NO: 2 - protein, 919 aa, Homo sapiens
  misc_feature: Incyte ID No 7103532CD1
MGVAGRNRPGAAWAVLLLLLLLPPLLLLAGAVPPGRGRAAGPQEDVDECAQGLDDCHADALCQNTPTSYKCSCKPGYQGEGRQCEDIDECGNELNGGCVHDCLNIPGNYRCTCFDGFMLAHDGHNCLDVDECLENNGGCQHTCVNVMGSYECCCKEGFFLSDNQHTCIHRSEEGLSCMNKDHGCSHICKEAPRGSVACECRPGFELAKNQRDCILTCNHGNGGCQHSCDDTADGPECSCHPQYKMHTDGRSCLEREDTVLEVTESNTTSVVDGDKRVKRRLLMETCAVNNGGCDRTCKDTSTGVHCSCPVGFTLQLDGKTCKDIDECQTRNGGCDHFCKNIVGSFDCGCKKGFKLLTDEKSCQDVDECSLDRTCDHSCINHPGTFACACNRGYTLYGFTHCGDVTTIRTSVTFKLNEGKCSLKNAELFPEGLRPALPEKHSSVKESFRYVNLTCSSGKQVPGAPGRPSTPKEMFITVEFELETNQKEVTASCDLSCIVKRTEKRLRKAIRTLRKAVHREQFHLQLSGMNLDVAKKPPRTSERQAESCGVGQGHAENQCVSCRAGTYYDGARERCILCPNGTFQNEEGQMTCEPCPRPGNSGALKTPEAWNMSECGGLCQPGEYSADGFAPCQLCALGTFQPEAGRTSCFPCGGGLATKHQGATSFQDCETRVQCSPGHFYNTTTHRCIRCPVGTYQPEFGKNNCVSCPGNSTTDFDGSTNITQCKNRRCGGELGDFTGYIESPNYPGNYPANTECTWTINPPPKRRILIVVPEIFLPIEDDCGDYLVMRKTSSSNSVTTYETCQTYERPIAFTSRSKKLWIQFKSNEGNSARGFQVPYVTYDEDYQELIEDIVRDGRLYASENHQEILKDKKLIKVLFDVLAHPQNYFKYTAQESREMFPRSFIRLLRPKVSRFLRPYK

SEQ ID NO: 3 - protein, 350 aa, Homo sapiens
  misc_feature: Incyte ID No 7500108CD1
MDTKLMCLLFFFSLPPLLVSNHTGRIKVVFTPSICKVTCTKGSCQNSCENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 4 - protein, 381 aa, Homo sapiens
  misc_feature: Incyte ID No 7500665CD1
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 5 - protein, 991 aa, Homo sapiens
  misc_feature: Incyte ID No 3569792CD1
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPGTWEEGAGELWRRKEEGLAVQAAPSFPLDSSSQRGLGRQAAVLSIKQRASFKIKDAKCRLHLRNKGKTEEAGSGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGGFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 6 - protein, 306 aa, Homo sapiens
  misc_feature: Incyte ID No 7500100CD1
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKGRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 7 - protein, 1668 aa, Homo sapiens
  misc_feature: Incyte ID No 5201851CD1
MAGAWLRWGLLLWAGLLASSAHGRLRRITYVVHPGPGLAAGALPLSGPPRSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 8 - protein, 504 aa, Homo sapiens
  misc_feature: Incyte ID No 7500667CD1
MAEAKTHWLGAALSLIPLIFLISGAEAASFQRNQLLQKEPDLRLENVQKFPSPEMIRALEYIENPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 9 - protein, 317 aa, Homo sapiens [sequence omitted]

SEQ ID NO: 10 - protein, 1721 aa, Homo sapiens
  misc_feature: Incyte ID No 7502082CD1
MAGAWLRWGLLLWAGLLASSAHGRLRRITYVVHPGPGLAAGALPLSGPPRSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 11 - protein, 1679 aa, Homo sapiens
  misc_feature: Incyte ID No 7502084CD1
MAGAWLRWGLLLWAGLLASSAHGRLRRITYVVHPGPGLAAGALPLSGPPRSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 12 - protein, 1626 aa, Homo sapiens
  misc_feature: Incyte ID No 7502085CD1
MAGAWLRWGLLLWAGLLASSAHGRLRRITYVVHPGPGLAAGALPLSGPPRSRTFNVALNARYSRSSAAAGAPSRASPGVPSERTRRTSKPGGAALQGLRPPPPPPPEPARPAVPGGQLHPNPGGHPAAAPFTKQGRQVVRSKVPQETQSGGGSRLQVHQKQQLQGVNVCGGRCCHGWSKAPGSQRCTKPSCVPPCQNGGMCLRPQLCVCKPGTKGKACETIAAQDTSSPVFGGQSPGAASSWGPPEQAAKHTSSKKADTLPRVSPVAQMTLTLKPKPSVGLPQQIHSQVTPLSSQSVVIHHGQTQEYVLKPKYFPAQKGISGEQSTEGSFPLRYVQDQVAAPFQLSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 13 - protein, 1300 aa, Homo sapiens
  misc_feature: Incyte ID No 7502093CD1
MDTKLMCLLFFFSLPPLLVSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 14 - protein, 1353 aa, Homo sapiens
  misc_feature: Incyte ID No 7502097CD1
MDTKLMCLLFFFSLPPLLVSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 15 - protein, 1342 aa, Homo sapiens
  misc_feature: Incyte ID No 7502108CD1
MDTKLMCLLFFFSLPPLLVSNHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDVNECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 16 - protein, 98 aa, Homo sapiens
  misc_feature: Incyte ID No 7500668CD1
MAEAKTHWLGAALSLIPLIFLISGAEAASFQRNQLLQKEPDLRLENVQKFPSPEMIRALEYIENLRQQAHKKESLSTCNSLLCMKRIPGITPLNAQMK

SEQ ID NO: 17 - protein, 133 aa, Homo sapiens
  misc_feature: Incyte ID No 7505114CD1
MFHVSFRYIFGLPPLILVLLPVASSDCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 18 - protein, 167 aa, Homo sapiens
  misc_feature: Incyte ID No 7506452CD1
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNVHPETKENEIYPVWSGLPSLQMADEESRLSAYYNLLHCLRRDSHKIDNYLKLLKCRIIHNNNC

SEQ ID NO: 19 - protein, 142 aa, Homo sapiens
  misc_feature: Incyte ID No 7506730CD1
MQLTRCCFVFLVQGSLYLVICGQDDGPPGSEDPERDDHEGQPRPRVPRKRGHISSKSRPMANSTLLGLLAPPGEAWGILGQPPNRPNHSPPPSAKVKKIFGWGDFYSNIKTVALNLLVTRNSRSSSKPRPPKSSTAGWSGRR

SEQ ID NO: 20 - protein, 212 aa, Homo sapiens
  misc_feature: Incyte ID No 7505046CD1
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIWIMLYKASIFFFFLKTGYEDKVPELYLILSGIKGKSITFANCPLHQLTSWVTTGRKSRSCSSWPINCIGPFGYAERRRKGGNQPSPVCPLGPSSHLSLDHISPWTLG

SEQ ID NO: 21 - protein, 75 aa, Homo sapiens
  misc_feature: Incyte ID No 7506453CD1
MNIKGSPWKGSLLLLLVSNLLLCQSVAPLPICPGGAARCQLPHFFPCHPRRQGASPTDESKRLSEPDSQHIAILE

SEQ ID NO: 22 - protein, 173 aa, Homo sapiens
  misc_feature: Incyte ID No 7509967CD1
MNIKGSPWKGSLLLLLVSNLLLCQSVAPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQLERTRTYKY

SEQ ID NO: 23 - DNA, 2598 bp, Homo sapiens
  misc_feature: Incyte ID No 7497502CB1
tggcaaaaattccccatcacaggaaacccgaaatcagaaaagttaagtcacccagggctggacccagactcttgcagctctcactttcacaatgcccttgggctgactaggctgcagaggggtttcacccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccgaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatccctcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcc

SEQ ID NO: 24 - DNA, 2914 bp, Homo sapiens
  misc_feature: Incyte ID No 7103532CB1
gggaccgtgtgaaaatgaggccggggctcggggggcgggcggggccgggccgggggtggcagcggcagcgggcaggcgtccgcgcacacctccccgcgccgccgccgccaccgcccgcactccgccgcctctgcccgcaaccgctgagccatccatgggggtcgcgggccgcaaccgtcccggggcggcctgggcggtgctgctgctgctgctgctgctgccgccactgctgctgctggcgggggccgtcccgccgggtcggggccgtgccgcggggccgcaggaggatgtagatgagtgtgcccaagggctagatgactgccatgccgacgccctgtgtcagaacacacccacctcctacaagtgctcctgcaagcctggctaccaaggggaaggcaggcagtgtgaggacatcgatgaatgtggaaatgagctcaatggaggctgtgtccatgactgtttgaatattccaggcaattatcgttgcacttgttttgatggcttcatgttggctcatgacggtcataattgtcttgatgtggacgagtgcctggagaacaatggcggctgccagcatacctgtgtcaacgtcatggggagctatgagtgctgctgcaaggaggggtttttcctgagtgacaatcagcacacctgcattcaccgctcggaagagggcctgagctgcatgaataaggatcacggctgtagtcacatctgcaaggaggccccaaggggcagcgtcgcctgtgagtgcaggcctggttttgagctggccaagaaccagagagactgcatcttgacctgtaaccatgggaacggtgggtgccagcactcctgtgacgatacagccgatggcccagagtgcagctgccatccacagtacaagatgcacacagatgggaggagctgccttgagcgagaggacactgtcctggaggtgacagagagcaacaccacatcagtggtggatggggataaacgggtgaaacggcggctgctcatggaaacgtgtgctgtcaacaatggaggctgtgaccgcacctgtaaggatacttcgacaggtgtccactgcagttgtcctgttggattcactctccagttggatgggaagacatgtaaagatattgatgagtgccagacccgcaatggaggttgtgatcatttctgcaaaaacatcgtgggcagttttgactgcggctgcaagaaaggatttaaattattaacagatgagaagtcttgccaagatgtggatgagtgctctttggataggacctgtgaccacagctgcatcaaccaccctggcacatttgcttgtgcttgcaaccgagggtacaccctgtatggcttcacccactgtggagatgtcaccaccatcaggacaagtgtaacctttaagctaaatgaaggcaagtgtagtttgaaaaatgctgagctgtttcccgagggtctgcgaccagcactaccagagaagcacagctcagtaaaagagagcttccgctacgtaaaccttacatgcagctctggcaagcaagtcccaggagcccctggccgaccaagcacccctaaggaaatgtttatcactgttgagtttgagcttgaaactaaccaaaaggaggtgacagcttcttgtgacctgagctgcatcgtaaagcgaaccgagaagcggctccgtaaagccatccgcacgctcagaaaggccgtccacagggagcagtttcacctccagctctcaggcatgaacctcgacgtggctaaaaagcctcccagaacatctgaacgccaggcagagtcctgtggagtgggccagggtcatgcagaaaaccaatgtgtcagttgcagggctgggacctattatgatggagcacgagaacgctgcattttatgtccaaatggaaccttccaaaatgaggaaggacaaatgacttgtgaaccatgcccaagaccaggaaattctggggccctgaagaccccagaagcttggaatatgtctgaatgtggaggtctgtgtcaacctggtgaatattctgcagatggctttgcaccttgccagctctgtgccctgggcacgttccagcctgaagctggtcgaacttcctgcttcccctgtggaggaggccttgccaccaaacatcagggagctacttcctttcaggactgtgaaaccagagttcaatgttcacctggacatttctacaacaccaccactcaccgatgtattcgttgcccagtgggaacataccagcctgaatttggaaaaaataattgtgtttcttgcccaggaaatagtacgactgactttgatggctccacaaacataacccagtgtaaaaacagaagatgtggaggggagctgggagatttcactgggtacattgaatccccaaactacccaggcaattacccagccaacaccgagtgtacgtggaccatcaacccaccccccaagcgccgcatcctgatcgtggtccctgagatcttcctgcccatagaggacgactgtggggactatctggtgatgcggaaaacctcttcatccaattctgtgacaacatatgaaacctgccagacctacgaacgccccatcgccttcacctccaggtcaaagaagctgtggattcagttcaagtccaatgaagggaacagcgctagagggttccaggtcccatacgtgacatatgatgaggactaccaggaactcattgaagacatagttcgagatggcaggctctatgcatctgagaaccatcaggaaatacttaaggataagaaacttatcaaggttctgtttgatgtcctggcccatccccagaactatttcaagtacacagcccaggagtcccgagagatgtttccaagatcgttcatccgattgctacgtcccaaagtgtccaggtttttgagaccttacaaatga

SEQ ID NO: 25 - DNA, 1458 bp, Homo sapiens
  misc_feature: Incyte ID No 7500108CB1
cctcttgctcctttcttttctttttttctgtttttttaaaccttccaaggcaagttcatggatactaagctgatgtgtttgttgttctttttctccctgcctccgctcctagtgagtaaccacactggccgcatcaaggtggtctttactccgagcatctgtaaagtgacctgcaccaagggcagctgtcagaacagctgtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacggaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaaaaaaaaaaaa

SEQ ID NO: 26 - DNA, 1703 bp, Homo sapiens
  misc_feature: Incyte ID No 7500665CB1
aagcagaggagctgtccgtgtgctgaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatataaaaaaaaaaaaaaaaaaaaagg

SEQ ID NO: 27 - DNA, 3202 bp, Homo sapiens
  misc_feature: Incyte ID No 3569792CB1
tccagccatgggctcggggcgcgtacccgggctctgcctgcttgtcctgctggtccacgcccgcgccgcccagtacagcaaagccgcgcaagatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaattgacatgcaactatggtaacggcggctgccagcacacgtgtgatgacacagagcagggtccccggtgcggctgccatatcaagtttgtgctccataccgacgggaagacatgcatcgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccaggtacatgggaggagggtgctggagagctttggaggagaaaagaggaaggactggccgttcaggcagctccttcattccccctggattcctccagccagcgggggttgggaaggcaggctgcagtgctgtccattaaacaacgggcctccttcaagatcaaggatgccaaatgccgtttgcacctgcgaaacaaaggcaaaacagaggaggctggcagtggtgccccctgctctgaatgccaggtcaccttcatccaccttaagtgtgactcctctcggaagggcaagggccgacgggcccggacccctccaggcaaagaggtcacaaggctcaccctggaactggaggcagaggtcagagccgaagaaaccacagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtcagctgcccgcagggaacgtattaccacggccagacggagcagtgtgtgccatgcccagcgggcaccttccaggagagagaagggcagctctcctgcgacctttgccctgggagtgatgcccacgggcctcttggagccaccaacgtcaccacgtgtgcaggtcagtgcccacctggccaacactctgtagatgggttcaagccctgtcagccatgcccacgtggcacctaccaacctgaagcaggacggaccctatgcttcccttgtggtgggggcctcaccaccaagcatgaaggggccatttccttccaagactgtgacaccaaagtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgggttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatagtaaccctaggctcagagacccaattttttaagcccccagactccttagccctcagagccggcagccccctaccctcagacaaggaactctctcctctctttttggagggaaaaaaaaaatatcactacacaaaccagcactctccctttctgtcacaggggcaaacaacgagaaacacaaaagaacccacaacaacaaccaaccacagaggagacaagc

SEQ ID NO: 28 - DNA, 1530 bp, Homo sapiens
  misc_feature: Incyte ID No 7500100CB1
gccactgcagtgctcgagccccgtgcaggggagcttgcgggaggatcgaccgacagacggacgcacgccgaggcactgcgccccccagccccgcgccggtgccaccgcagcccgaccccggccgccagtccagccgcccctcgcccggtgcctaggtgcccggccccacaccgccagctgctcggcgcccgggtccgccatgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaaggtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggatgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctgtatgaactttatctaaagaaaaataaatctgttctgggctcaaaaaaaaaaaa

SEQ ID NO: 29 - DNA, 5894 bp, Homo sapiens
  misc_feature: Incyte ID No 5201851CB1
atggcgggggcctggctcaggtgggggctcctgctctgggcagggctcctcgcgtcctcggcgcacggccggctgcggaggatcacctacgtggtgcacccgggccccggcctggcagccggcgccttgcccctgagcgggcccccgcgttcgcggacattcaacgtcgcgctcaacgccaggtacagccgcagctcggcggctgccggcgcccccagccgtgcctcccccggggtcccctcggagaggacccggcgcacgagcaagccgggcggcgcggccctgcaggggctcagaccgccgccgccgccgccgccggagcctgcgcgtcccgcggtccccggcgggcagctccaccccaatcccggcggccacccggcagccgccccgttcaccaaacaaggcaggcaagttgtgcgctccaaggtgccgcaggagacccagagcggcggaggctctaggctgcaggttcaccagaagcagcagctgcagggggtcaatgtctgtggagggcggtgctgtcatggctggagtaaggcccctggctcccagaggtgcaccaaacctagctgtgttccgccatgtcagaatggagggatgtgtctccggccacaactctgtgtgtgtaaaccagggaccaagggcaaagcctgtgaaacaatagctgcccaggacacctcgtcaccagtctttggagggcagagtcctggggctgcttcctcgtggggccctcctgagcaagcagcaaagcatacttcatctaagaaggcagacactctaccaagagtcagccctgtggcccagatgaccttaaccctcaagccgaagccttcagtgggactcccccagcagatacattctcaagtgactcctctttcttcccagagtgtggtgattcaccatggccagacccaggaatacgtgctcaagcccaagtactttccagcccagaaggggatttcaggagagcagtccactgaaggttctttccctttaagatatgtgcaggatcaagttgcggcaccttttcagctgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcgttggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatattgatgaatgtgtaaacaacactgtttgtgacagtcacgggttttgtgacaatacagctggctccttccgctgcctctgttatcagggctttcaagccccacaggatgggcaagggtgtgtggatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaagtactttatattttcagtgttttgtctgattttaatgtccgttcttagccaagctgctagcaggtgttaattggatccctttccttcactgaaatggaagagtttataagcttacgttagtattgtaatatgtaaagtaagcccaacaaaaatttttaaaaatttgatgatccccaatatatctaccattgtatgttaaataaatcaccatttttgtagaaaaaaattctacctgagagtaattgtcaatgagtacatgtgtataagttgtatcccactctccccacttttatc

SEQ ID NO: 30 - DNA, 2031 bp, Homo sapiens
  misc_feature: Incyte ID No 7500667CB1
gaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatataaaaaaaaaaaaaaaaaa

SEQ ID NO: 31 - DNA, 1617 bp, Homo sapiens
  misc_feature: Incyte ID No 7744055CB1
  unsure 1526: a, t, c, g, or other
gcacatgcccggcagaagtccgggcgcgcaacttcgcagaacctcactgcccgtccctcctcgcctcagtctcctctgtcctctcccaggcaagaggaccggcggaggcacctctctcgagtcttaggctgcggaatctaagactcagcgagaggagcccgggaggagacagaactttccccttttttcccatcccttcttcttgctcagagaggcaagcaaggcgcggagctttagaaagttcttaagtggtcaggaaggtaggtgcttccctttttctcctcacaaggaggtgaggctgggacctccgggccagcttctcacctcatagggtgtacctttcccggctccagcagccaatgtgcttcggagccgctctctgcagagccagagggcaggccggcttctcggtgtgtgcctaagaggatggatcggaggtcccgggctcagcagtggcgccgagctcgccataattacaacgacctgtgcccgcccataggccgccgggcagccaccgcgctcctctggctctcctgctccatcgcgctcctccgcgcccttgccacctccaacgcccgtgcccagcagcgcgcggctgcccaacagcgccggagcttccttaacgcccaccaccgctccggcgcccaggtattccctgagtcccccgaatcggaatctgaccacgagcacgaggaggcagaccttgagctgtccctccccgagtgcctagagtacgaggaagagttcgactacgagaccgagagcgagaccgagtccgaaatcgagtccgagaccgacttcgagaccgagcctgagaccgcccccaccactgagcccgagaccgagcctgaagacgatcgcggcccggtggtgcccaagcactccaccttcggccagtccctcacccagcgtctgcacgctctcaagttgcgaagccccgacgcctccccaagtcgcgcgccgcccagcactcaggagccccagagccccagggaaggggaggagctcaagcccgaggacaaagatccaagggaccccgaagagtcgaaggagcccaaggaggagaagcagcggcgtcgctgcaagccaaagaagcccacccgccgtgacgcgtccccggagtccccttccaaaaagggacccatcccccatccggcgtcactaatggaggacgccgtccagattctccttgttttcatggattcaggtgctggagaatctggtaaaagcaccattgtgaagcagatgaggatcctgcatgttaatgggtttaatggagagggcggcgaagaggacccgcaggctgcaaggagcacagcgatggcagtgagaaggcaacccaagtgcaggacatcaaaacaacctgaaagaggcgattgaaaccattgtggccgccatgagcaacctggtgccccccgtggagctggccaaccccgagaaccagttcagagtggactacattctgagtgtgatgaacgtgcctgactttgacttccctcccgaattctatgagcatgccaggctcntgtgggaggatgaaggagtgcgtgcctgctacgaacgctcccacgaggtaccagctgattgactgtgcccacaaccttccggggacaagat

SEQ ID NO: 32 - DNA, 5758 bp, Homo sapiens
  misc_feature: Incyte ID No 7502082CB1
atggcgggggcctggctcaggtgggggctcctgctctgggcagggctcctcgcgtcctcggcgcacggccggctgcggaggatcacctacgtggtgcacccgggccccggcctggcagccggcgccttgcccctgagcgggcccccgcgttcgcggacattcaacgtcgcgctcaacgccaggtacagccgcagctcggcggctgccggcgcccccagccgtgcctcccccggggtcccctcggagaggacccggcgcacgagcaagccgggcggcgcggccctgcaggggctcagaccgccgccgccgccgccgccggagcctgcgcgtcccgcggtccccggcgggcagctccaccccaatcccggcggccacccggcagccgccccgttcaccaaacaaggcaggcaagttgtgcgctccaaggtgccgcaggagacccagagcggcggaggctctaggctgcaggttcaccagaagcagcagctgcagggggtcaatgtctgtggagggcggtgctgtcatggctggagtaaggcccctggctcccagaggtgcaccaaacctagctgtgttccgccatgtcagaatggagggatgtgtctccggccacaactctgtgtgtgtaaaccagggaccaagggcaaagcctgtgaaacaatagctgcccaggacacctcgtcaccagtctttggagggcagagtcctggggctgcttcctcgtggggccctcctgagcaagcagcaaagcatacttcatctaagaaggcagacactctaccaagagtcagccctgtggcccagatgaccttaaccctcaagccgaagccttcagtgggactcccccagcagatacattctcaagtgactcctctttcttcccagagtgtggtgattcaccatggccagacccaggaatacgtgctcaagcccaagtactttccagcccagaaggggatttcaggagagcagtccactgaaggttctttccctttaagatatgtgcaggatcaagttgcggcaccttttcagctgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcgttggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagctgcttttaaggaaatctgtcctggtggaatgggttatacggtttctggcgttcatagacgcaggccaatccatcaccatgtaggtaaaggacctgtatttgtcaagccaaagaacactcaacctgttgctaaaagtactcatcctccacctctcccagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccgcaaggggagtgcctaaacacagagggttctttccattgtgtctgccagcagggtttctcaatctctgcagatggccgtacgtgtgaagatattgatgaatgtgtaaacaacactgtttgtgacagtcacgggttttgtgacaatacagctggctccttccgctgcctctgttatcagggctttcaagccccacaggatgggcaagggtgtgtggatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacggaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcagattgtctgtcctgtggagcaagcagtgattttgttccaaactttgtataccatttggaggaaag

SEQ ID NO: 33 - DNA, 5292 bp, Homo sapiens
  misc_feature: Incyte ID No 7502084CB1
atggcgggggcctggctcaggtgggggctcctgctctgggcagggctcctcgcgtcctcggcgcacggccggctgcggaggatcacctacgtggtgcacccgggccccggcctggcagccggcgccttgcccctgagcgggcccccgcgttcgcggacattcaacgtcgcgctcaacgccaggtacagccgcagctcggcggctgccggcgcccccagccgtgcctcccccggggtcccctcggagaggacccggcgcacgagcaagccgggcggcgcggccctgcaggggctcagaccgccgccgccgccgccgccggagcctgcgcgtcccgcggtccccggcgggcagctccaccccaatcccggcggccacccggcagccgccccgttcaccaaacaaggcaggcaagttgtgcgctccaaggtgccgcaggagacccagagcggcggaggctctaggctgcaggttcaccagaagcagcagctgcagggggtcaatgtctgtggagggcggtgctgtcatggctggagtaaggcccctggctcccagaggtgcaccaaacctagctgtgttccgccatgtcagaatggagggatgtgtctccggccacaactctgtgtgtgtaaaccagggaccaagggcaaagcctgtgaaacaatagctgcccaggacacctcgtcaccagtctttggagggcagagtcctggggctgcttcctcgtggggccctcctgagcaagcagcaaagcatacttcatctaagaaggcagacactctaccaagagtcagccctgtggcccagatgaccttaaccctcaagccgaagccttcagtgggactcccccagcagatacattctcaagtgactcctctttcttcccagagtgtggtgattcaccatggccagacccaggaatacgtgctcaagcccaagtactttccagcccagaaggggatttcaggagagcagtccactgaaggttctttccctttaagatatgtgcaggatcaagttgcggcaccttttcagctgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcgttggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagctgcttttaaggaaatctgtcctggtggaatgggttatacggtttctggcgttcatagacgcaggccaatccatcaccatgtaggtaaaggacctgtatttgtcaagccaaagaacactcaacctgttgctaaaagtactcatcctccacctctcccagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctgaaaatacggaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtg

SEQ ID NO: 34 - DNA, 5549 bp, Homo sapiens
  misc_feature: Incyte ID No 7502085CB1
atggcgggggcctggctcaggtgggggctcctgctctgggcagggctcctcgcgtcctcggcgcacggccggctgcggaggatcacctacgtggtgcacccgggccccggcctggcagccggcgccttgcccctgagcgggcccccgcgttcgcggacattcaacgtcgcgctcaacgccaggtacagccgcagctcggcggctgccggcgcccccagccgtgcctcccccggggtcccctcggagaggacccggcgcacgagcaagccgggcggcgcggccctgcaggggctcagaccgccgccgccgccgccgccggagcctgcgcgtcccgcggtccccggcgggcagctccaccccaatcccggcggccacccggcagccgccccgttcaccaaacaaggcaggcaagttgtgcgctccaaggtgccgcaggagacccagagcggcggaggctctaggctgcaggttcaccagaagcagcagctgcagggggtcaatgtctgtggagggcggtgctgtcatggctggagtaaggcccctggctcccagaggtgcaccaaacctagctgtgttccgccatgtcagaatggagggatgtgtctccggccacaactctgtgtgtgtaaaccagggaccaagggcaaagcctgtgaaacaatagctgcccaggacacctcgtcaccagtctttggagggcagagtcctggggctgcttcctcgtggggccctcctgagcaagcagcaaagcatacttcatctaagaaggcagacactctaccaagagtcagccctgtggcccagatgaccttaaccctcaagccgaagccttcagtgggactcccccagcagatacattctcaagtgactcctctttcttcccagagtgtggtgattcaccatggccagacccaggaatacgtgctcaagcccaagtactttccagcccagaaggggatttcaggagagcagtccactgaaggttctttccctttaagatatgtgcaggatcaagttgcggcaccttttcagctgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcattggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggaattgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaagtactttatattttcagtgttttgtctgattttaatgtccgttcttagccaaagctgctagcaggtgttaattgg

SEQ ID NO: 35 - DNA, 4741 bp, Homo sapiens
  misc_feature: Incyte ID No 7502093CB1
gtgcagcattgtggttagtaatcccactccagtgactcgacttcaaatgtggttttggagtgcatcccagagttctgtttggtaagcttcctactcctgtttcagagacaccactgaatacagagcagcgagcactgaaggcttccctctttccttaaacctgtcgggttgtgggctctctcttttcccctcttgctcctttcttttctttttttctgtttttttaaaccttccaaggcaagttcatggatactaagctgatgtgtttgttgttctttttctccctgcctccgctcctagtgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcattggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaa

SEQ ID NO: 36 - DNA, 4900 bp, Homo sapiens
  misc_feature: Incyte ID No 7502097CB1
gtgcagcattgtggttagtaatcccactccagtgactcgacttcaaatgtggttttggagtgcatcccagagttctgtttggtaagcttcctactcctgtttcagagacaccactgaatacagagcagcgagcactgaaggcttccctctttccttaaacctgtcgggttgtgggctctctcttttcccctcttgctcctttcttttctttttttctgtttttttaaaccttccaaggcaagttcatggatactaagctgatgtgtttgttgttctttttctccctgcctccgctcctagtgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcattggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagctgcttttaaggaaatctgtcctggtggaatgggttatacggtttctggcgttcatagacgcaggccaatccatcaccatgtaggtaaaggacctgtatttgtcaagccaaagaacactcaacctgttgctaaaagtactcatcctccacctctcccagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaa

SEQ ID NO: 37 - DNA, 4942 bp, Homo sapiens
  misc_feature: Incyte ID No 7502108CB1
gtgcagcattgtggttagtaatcccactccagtgactcgacttcaaatgtggttttggagtgcatcccagagttctgtttggtaagcttcctactcctgtttcagagacaccactgaatacagagcagcgagcactgaaggcttccctctttccttaaacctgtcgggttgtgggctctctcttttcccctcttgctcctttcttttctttttttctgtttttttaaaccttccaaggcaagttcatggatactaagctgatgtgtttgttgttctttttctccctgcctccgctcctagtgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcattggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgcaccctctgtctgttcacctcaccaagcagctctgctgttgtagtgtgggcaaggcctggggcccacactgtgagaaatgtccccttccaggcacagccaaggaagagccagtggaggccctgaccttctcccgggaacacgggccaggagtggcggagccagaagtggcaactgcaccccctgaaaaggaaataccttcattggatcaagagaaaaccaaacttgagcctggtcaaccccagctgtctccaggcatttccactattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccacaaggggagtgcctaaacacagagggttctttccattgtgtctgccagcagggtttctcaatctctgcagatggccgtacgtgtgaagatgtgaatgaatgtgtaaacaacactgtttgtgacagtcacgggttttgtgacaatacagctggctccttccgctgcctctgttatcagggctttcaagccccacaggatgggcaagggtgtgtggatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgctttgatgggtatcacttggatacggccaagatgacctgtgtcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggaattgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgtttttttactattgcttgattaaatttggcatttaaatagtggtggaaatattttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaagtactttatattttcagtgttttgtctgattttaatgtccgttcttagccaaagctgctagcaggtgttaattgg

SEQ ID NO: 38 - DNA, 2144 bp, Homo sapiens
  misc_feature: Incyte ID No 7500668CB1
tcggctcgaggctgtccgtgtgctgaaacggcccgagaagctcgcccggagaacggggaggaatatgctgtggagctcctctgccatataaacaaaaagaggaaatctttcaaacatggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagtgttttgtggagtactggaccaaaaaaataaagcattataaatataaaaaaaaaaaaaaa

SEQ ID NO: 39 - DNA, 1216 bp, Homo sapiens
  misc_feature: Incyte ID No 7505114CB1
gtaagaggaaccagctgcagagatcaccctgcccaacacagactcggcaactccgcggaagaccagggtcctgggagtgactatgggcggtgagagcttgctcctgctccagttgcggtcatcatgactacgcccgcctcccgcagaccatgttccatgtttcttttaggtatatctttggacttcctcccctgatccttgttctgttgccagtagcatcgtctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactgaaaaatatggagtggcaatatagaaacacgaactttagctgcatcctccaagaatctatctgcttatgcagtttttcagagtggaatgcttcctagaagttactgaatgcaccatggtcaaaacggattagggcatttgagaaatgcatattgtattactagaagatgaatacaaacaatggaaactgaatgctccagtcaacaaactatttcttatatatgtgaacatttatcaatcagtataattctgtactgatttttgtaagacaatccatgtaaggtatcagttgcaataatacttctcaaacctgtttaaatatttcaagacattaaatctatgaagtatataatggtttcaaagattcaaaattgacattgctttactgtcaaaataattttatggctcactatgaatctattatactgtattaagagtgaaaattgtcttcttctgtgctggagatgttttagagttaacaatgatatatggataatgccggtgagaataagagagtcataaaccttaagtaagcaacagcataacaaggtccaagatacctaaaagagatttcaagagatttaattaatcatgaatgtgtaacacagtgccttcaataaatggtatagcaaatgttttgacatgaaaaaaggacaatttcaaaaaaa

SEQ ID NO: 40 - DNA, 818 bp, Homo sapiens
  misc_feature: Incyte ID No 7506452CB1
gcattcggctcgagcaaagacagagacaccaagaagaatcggaacatacaggctttgatatcaaaggtttataaagccaatatctgggaaagagaaaaccgtgagacttccagatcttctctggtgaagtgtgtttcctgcaacgatcacgaacatgaacatcaaaggatcgccatggaaagggtccctcctgctgctgctggtgtcaaacctgctcctgtgccagagcgtggcccccttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatgttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgctaagcccacatccatttcatctatttctgagaaggtccttaatgatccgttccattgcaagcttcttttagttgtatctcttttgaatccatgcttgggtgtaacaggtctcctcttaaaaaataaaaactgactccttagagacatcaaaatctaaaaaaaa

SEQ ID NO: 41 - DNA, 833 bp, Homo sapiens
  misc_feature: Incyte ID No 7506730CB1
ggggactggagcatgggacggcgcgcctgaaggagcaggaaggggaaggaggcctgggaccccgaaaagagaaggggagagcgaggggacgagagcggaggaggaagatgcaactgactcgctgctgcttcgtgttcctggtgcagggtagcctctatctggtcatctgtggccaggatgatggtcctcccggctcagaggaccctgagcgtgatgaccacgagggccagccccggccccgggtgcctcggaagcggggccacatctcatctaagtcccgccccatggccaattccactctcctagggctgctggccccgcctggggaggcttggggcattcttgggcagccccccaaccgcccgaaccacagccccccaccctcagccaaggtgaagaaaatctttggctggggcgacttctactccaacatcaagacggtggccctgaacctgctcgtcaccaggaacagcagatcttcatcgaagccaaggcctccaaaatcttcaactgccggatggagtgggagaaggtagaacggggccgccggacctcgctttgcacccacgacccagccaagatctgctcccgagaccacgctcagagctcagccacctggagctgctcccagcccttcaaagtcgtctgtgtctacatcgccttctacagcacggactatcggctggtccagaaggtgtgcccagattacaactaccatagtgataccccctactacccatctgggtgacccggggcaggccacagaggccaggccagggctggaaggacaggcctgcccatgcaggagaccatctggacaccgggcagggaag

SEQ ID NO: 42 - DNA, 1505 bp, Homo sapiens
  misc_feature: Incyte ID No 7505046CB1
cccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatctggattatgttatacaaggcaagcattttttttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggttcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtgtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggggggggggggcccacaatcagaagatacatccgtcaaaacgagggaaaatatatcaacaagggacagaactaaaaaagggagcggggataaaaaaataaaaaaaaacaataaaacaaaaaaacaacaagaagggaggcgagaaacaaaaaaaacaacaataaggaaataaaaaacaacaacaagagaagtatacaaaaaacgtagtagaaaca

SEQ ID NO: 43 - DNA, 889 bp, Homo sapiens
  misc_feature: Incyte ID No 7506453CB1
gtacctcaaagacagagacaccaagaagaatcggaacatacaggctttgatatcaaaggtttataaagccaatatctgggaaagagaaaaccgtgagacttccagatcttctctggtgaagtgtgtttcctgcaacgatcacgaacatgaacatcaaaggatcgccatggaaagggtccctcctgctgctgctggtgtcaaacctgctcctgtgccagagcgtggcccccttgcccatctgtcccggcggggctgcccgatgccagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgctaagcccacatccatttcatctatttctgagaaggtccttaatgatccgttccattgcaagcttcttttagttgtatctcttttgaatccatgcttgggtgtaacaggtctcctcttaaaaaataaaaactgactccttagagacatcaaaatctaaaaaaaaaaaaaaagcggccgctcgcgatctagaactagc

SEQ ID NO: 44 - DNA, 1066 bp, Homo sapiens
  misc_feature: Incyte ID No 7509967CB1
gtacctcaaagacagagacaccaagaagaatcggaacatacaggctttgatatcaaaggtttataaagccaatatctgggaaagagaaaaccgtgagacttccagatcttctctggtgaagtgtgtttcctgcaacgatcacgaacatgaacatcaaaggatcgccatggaaagggtccctcctgctgctgctggtgtcaaacctgctcctgtgccagagcgtggcccccttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccagttagaaagaacaaggacatacaaatactaataatatgaagaataagtcactctttttttgtgtgatgaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgctaagcccacatccatttcatctatttctgagaaggtccttaatgatccgttccattgcaagcttcttttagttgtatctcttttgaatccatgcttgggtgtaacaggtctcctcttaaaaaataaaaactgactccttagagacatcaaaatctaaaaaaaaaaaaaaa

(19) United States
(12) Patent Application Publication
(10) Pub. No.: US 2007/0225218 A1
(43) Pub. Date: Sep. 27, 2007

(54) Extracellular Messengers

(76) Inventors: Angelo M Delegeane, Milpitas, CA (US); Mark L Borowsky, Northampton, MA (US); Farrah A. Khan, Canton, MI (US); Liam Kearney, San Francisco, CA (US); Jayalaxmi Ramkumar, Fremont, CA (US); Narinder K. Chawla, Union City, CA (US); Yan Lu, Mountain View, CA (US); Cynthia D. Honchell, San Francisco, CA (US); Deborah A kallick, Galveston, TX (US); Brooke M Emerling, Chicago, IL (US); Ann E. Gorvard, Bellingham, WA (US); Jennifer A Griffin, Fremont, CA (US); Bridget A. Warren, San Marcos, CA (US); Henry Yue, Sunnvale, CA (US); Kavitha Thangavelu, Sunnyvale, CA (US); William W. Sprague, Sacramento, CA (US); Craig H Ison, San Jose, CA (US); Vicki S Elliott, San Jose, CA (US); Patricia M Lehr-Mason, Morgan Hill, CA (US); Thomas W Richardson, Redwood City, CA (US); Uyen K Tran, San Jose, CA (US); Anita Swarnakar, San Francisco, CA (US); Pei Jin, Palo Alto, CA (US); Amy E Kable, Silver Spring, MD (US)

Correspondence Address: FOLEY AND LARDNER LLP;SUITE 500, 3000 K STREET NW, WASHINGTON, DC, 20007, US

(21) Appl. No.: 10/481,700

(22) PCT Filed: Jun. 26, 2002
(86) PCT No.: PCT/US02/20430

§ 371 Date: Oct. 19, 2004

Related U.S. Application Data
(60) Provisional application No. 60/301,789, filed on Jun. 29, 2001.
(60) Provisional application No. 60/324,149, filed on Sep. 21, 2001.
(60) Provisional application No. 60/327,713, filed on Oct. 5, 2001.
(60) Provisional application No. 60/329,215, filed on Oct. 12, 2001.
(60) Provisional application No. 60/340,218, filed on Dec. 14, 2001.
(60) Provisional application No. 60/370,761, filed on Apr. 5, 2002.
(60) Provisional application No. 60/373,824, filed on Apr. 19, 2002.

(51) Int. Cl. C12P 21/00; A61K 38/00; C07H 21/04; C07K 14/00; C12Q 1/68; G01N 33/53; C07K 16/18; C12N 15/00; C12N 5/06

(52) U.S. Cl. 514/12; 435/320.1; 435/325; 435/6; 435/69.1; 435/7.1; 530/350; 530/387.1; 536/23.1

(57) ABSTRACT

Various embodiments of the invention provide human extracellular messengers (EXMES) and polynucleotides which identify and encode EXMES. Embodiments of the invention also provide expression vectors, host cells, antibodies, agonists, and antagonists. Other embodiments provide methods for diagnosing, treating, or preventing disorders associated with aberrant expression of EXMES.

### TECHNICAL FIELD

The invention relates to novel nucleic acids, extracellular messengers encoded by these nucleic acids, and to the use of these nucleic acids and proteins in the diagnosis, treatment, and prevention of autoimmune/inflammatory disorders, neurological disorders; endocrine disorders; developmental disorders; cell proliferative disorders including cancer; reproductive disorders; cardiovascular disorders; and infections. The invention also relates to the assessment of the effects of exogenous compounds on the expression of nucleic acids and extracellular messengers.

### BACKGROUND OF THE INVENTION

Intercellular communication is essential for the growth and survival of multicellular organisms, and in particular, for the function of the endocrine, nervous, and immune systems. In addition, intercellular communication is critical for developmental processes such as tissue construction and organogenesis, in which cell proliferation, cell differentiation, and morphogenesis must be spatially and temporally regulated in a precise and coordinated manner. Cells communicate with one another through the secretion and uptake of diverse types of signaling molecules such as hormones, growth factors, neuropeptides, and cytokines.

Hormones

Hormones are signaling molecules that coordinately regulate basic physiological processes from embryogenesis throughout adulthood. These processes include metabolism, respiration, reproduction, excretion, fetal tissue differentiation and organogenesis, growth and development, homeostasis, and the stress response. Hormonal secretions and the nervousss are tightly integrated and interdependent. Hormones are secreted by endocrine glands, primarily the hypothalamus and pituitary, the thyroid and parathyroid, the pancreas, the adrenal glands, and the ovaries and testes.

The secretion of hormones into the circulation is tightly controlled. Hormones are often secreted in diurnal, pulsatile, and cyclic patterns. Hormone secretion is regulated by perturbations in blood biochemistry, by other upstream-acting hormones, by neural impulses, and by negative feedback loops. Blood hormone concentrations are constantly monitored and adjusted to maintain optimal, steady-state levels. Once secreted, hormones act only on those target cells that express specific receptors.

Most disorders of the endocrine system are caused by either hyposecretion or hypersecretion of hormones. Hyposecretion often occurs when a hormone's gland of origin is damaged or otherwise impaired. Hypersecretion often results from the proliferation of tumors derived from hormone-secreting cells. Inappropriate hormone levels may also be caused by defects in regulatory feedback loops or in the processing of hormone precursors. Endocrine malfunction may also occur when the target cell fails to respond to the hormone.

Hormones can be classified biochemically as polypeptides, steroids, eicosanoids, or amines. Polypeptides, which include diverse hormones such as insulin and growth hormone, vary in size and function and are often synthesized as inactive precursors that are processed intracellularly into mature, active forms. Amines, which include epinephrine and dopamine, are amino acid derivatives that function in neuroendocrine signaling. Steroids, which include the cholesterol-derived hormones estrogen and testosterone, function in sexual development and reproduction. Eicosanoids, which include prostaglandins and prostacyclins, are fatty acid derivatives that function in a variety of processes. Most polypeptides and some amines are soluble in the circulation where they are highly susceptible to proteolytic degradation within seconds after their secretion. Steroids and lipids are insoluble and must be transported in the circulation by carrier proteins. The following discussion will focus primarily on polypeptide hormones.

Hormones secreted by the hypothalamus and pituitary gland play a critical role in endocrine function by regulating hormonal secretions from other endocrine glands in response to neural signals. Hypothalamic hormones include thyrotropin-releasing hormone, gonadotropin-releasing hormone, somatostatin, growth-hormone releasing factor, corticotropin-releasing hormone, substance P, dopamine, and prolactin-releasing hormone. These hormones directly regulate the secretion of hormones from the anterior lobe of the pituitary. Hormones secreted by the anterior pituitary include adrenocorticotropic hormone (ACTH), melanocyte-stimulating hormone, somatotropic hormones such as growth hormone and prolactin, glycoprotein hormones such as thyroid-stimulating hormone, luteinizing hormone (LH), and follicle-stimulating hormone (FSH), β-lipotropin, and β endorphins. These hormones regulate hormonal secretions from the thyroid, pancreas, and adrenal glands, and act directly on the reproductive organs to stimulate ovulation and spermatogenesis. The posterior pituitary synthesizes and secretes antidiuretic hormone (ADH, vasopressin) and oxytocin.

Disorders of the hypothalamus and pituitary often result from lesions such as primary brain tumors, adenomas, infarction associated with pregnancy, hypophysectomy, aneurysms, vascular malformations, thrombosis, infections, immunological disorders, and complications due to head trauma. Such disorders have profound effects on the function of other endocrine glands. Disorders associated with hypopituitarism include hypogonadism, Sheehan syndrome, diabetes insipidus, Kallman's disease, Hand-Schuller-Christian disease, Letterer-Siwe disease, sarcoidosis, empty sella syndrome, and dwarfism. Disorders associated with hyperpituitarism include acromegaly, giantism, and syndrome of inappropriate ADH secretion (SIADH), often caused by benign adenomas.

Hormones secreted by the thyroid and parathyroid primarily control metabolic rates and the regulation of serum calcium levels, respectively. Thyroid hormones include calcitonin, somatostatin, and thyroid hormone. The parathyroid secretes parathyroid hormone. Disorders associated with hypothyroidism include goiter, myxedema, acute thyroiditis associated with bacterial infection, subacute thyroiditis associated with viral infection, autoimmune thyroiditis (Hashimoto's disease), and cretinism. Disorders associated with hyperthyroidism include thyrotoxicosis and its various forms, Grave's disease, pretibial myxedema, toxic multinodular goiter, thyroid carcinoma, and Plummer's disease. Disorders associated with hyperparathyroidism include Conn disease (chronic hypercalemia) leading to bone resorption and parathyroid hyperplasia.

Hormones secreted by the pancreas regulate blood glucose levels by modulating the rates of carbohydrate, fat, and protein metabolism. Pancreatic hormones include insulin, glucagon, amylin, γ-aminobutyric acid, gastrin, somatostatin, and pancreatic polypeptide. The principal disorder associated with pancreatic dysfunction is diabetes mellitus caused by insufficient insulin activity. Diabetes mellitus is generally classified as either Type I (insulin-dependent, juvenile diabetes) or Type II (non-insulin-dependent, adult diabetes). The treatment of both forms by insulin replacement therapy is well known. Diabetes mellitus often leads to acute complications such as hypoglycemia (insulin shock), coma, diabetic ketoacidosis, lactic acidosis, and chronic complications leading to disorders of the eye, kidney, skin, bone, joint, cardiovascular system, nervous system, and to decreased resistance to infection.

The anatomy, physiology, and diseases related to hormonal function are reviewed in McCance, K. L. and S. E. Huether (1994) Pathophysiology: The Biological Basis for Disease in Adults and Children, Mosby-Year Book, Inc., St. Louis, Mo.; Greenspan, F. S. and J. D. Baxter (1994) Basic and Clinical Endocrinology, Appleton and Lange, East Norwalk, Conn.

Growth Factors

Growth factors are secreted proteins that mediate intercellular communication. Unlike hormones, which travel great distances via the circulatory system, most growth factors are primarily local mediators that act on neighboring cells. Most growth factors contain a hydrophobic N-terminal signal peptide sequence which directs the growth factor into the secretory pathway. Most growth factors also undergo post-translational modifications within the secretory pathway. These modifications can include proteolysis, glycosylation, phosphorylation, and intramolecular disulfide bond formation. Once secreted, growth factors bind to specific receptors on the surfaces of neighboring target cells, and the bound receptors trigger intracellular signal transduction pathways. These signal transduction pathways elicit specific cellular responses in the target cells. These responses can include the modulation of gene expression and the stimulation or inhibition of cell division, cell differentiation, and cell motility.

Growth factors fall into at least two broad and overlapping classes. The broadest class includes the large polypeptide growth factors, which are wide-ranging in their effects. These factors include epidermal growth factor (EGF), fibroblast growth factor (FGF), transforming growth factor-β (TGF-β), insulin-like growth factor (IGF), nerve growth factor (NGO), and platelet-derived growth factor (PDGF), each defining a family of numerous related factors. The large polypeptide growth factors, with the exception of NGF, act as mitogens on diverse cell types to stimulate wound healing, bone synthesis and remodeling, extracellular matrix synthesis, and proliferation of epithelial, epidermal, and connective tissues. Members of the TGF-β, EGF, and FGF families also function as inductive signals in the differentiation of embryonic tissue. NGF functions specifically as a neurotrophic factor, promoting neuronal growth and differentiation.

Some of the large polypeptide growth factors carry out specific functions on a restricted set of target tissues. For example, mouse growth/differentiation factor 9 (GDF-9) is a TGF-β family member that is expressed solely in the ovary (McPherron, A. C. and S.-J. Lee (1993) J. Biol. Chem. 268:3444-3449). NGF functions specifically as a neurotrophic factor, promoting neuronal growth and differentiation. Scubel (signal peptide-CUB domain-EGF-related 1) may play roles in the development of several organ systems. The protein, which contains ten EGF repeats and a CUB domain, is expressed in the developing central nervous system, gonads, somites, surface ectoderm, and limb buds (Grimmond et al. (2000) Genomics 70:74-81).

Hepatocyte growth factor (HGF) promotes cell growth, cell motility and mophogenesis in various target tissues (Michalopoulos, G. K. and Zarnegar, R. (1992) Hepatology 15:149-155; Michalopoulos and DeFrances, M. C. (1997) Science 276:60-66). HGF is required for liver and placental development in mice, and stimulates the renewal of cells in adult organs, including liver, lung, and kidney (Schmidt, C. et al. (1995) Nature 373:699-702). HGF contains four kringle domains followed by a serine protease-like domain, and mediates its effects through binding and activation of c-met, a tyrosine kinase receptor.

Follistatin (FS) is a protein that specifically binds and inhibits activin, a member of the transforming growth factor-β family of growth and differentiation factors. Activin performs a variety of functions associated with growth and differentiation, including induction of mesoderm in the developing embryo and regulation of female sex hormone secretion in the adult (de Krester, D. M. (1998) J. Reprod. Immunol. 39:1-12). Both activin and FS are found in many types of cells. The interaction of FS and activin influences a variety of cellular processes in the gonadal tissues, the pituitary gland, membranes associated with pregnancy, the vascular tissues, and the liver (reviewed in Phillips, D. J. and D. M. de Krester (1998) Front. Neuroendocrinol. 19:287-322). FS may also play a direct role in the neuralization of embryonic tissue (Hemnmati-Brivanlou et al. (1994) Cell 77:283-295).

FS is conserved among diverse species such as frog, chicken, and human. Variants of human FS include a 288 amino acid and a 315 amino acid isoform (McConnell, D. S. et al. (1998) J. Clin. Endocrinol. Metab. 83:851-858). Most follistatins contain a conserved domain with ten regularly spaced cysteine residues. These residues are likely involved in disulfide bond formation and the binding of cations. Similar domains are observed in Kazal protease inhibitors and osteonectin (also called SPARC or BM-40), an extracellular matrix-associated glycoprotein expressed in a variety of tissues during embryogenesis and repair (reviewed in Lane, T. F. and E. H. Sage (1994) FASEB J. 8:163-173). Osteonectin contains not only an FS-like polycysteine domain, but also other modular domains that can function independently to bind cells and matrix components and can change cell shape by selectively disrupting cellular contacts with matrix. High levels of osteonectin are associated with developing bones and teeth, principally osteoblasts, odontoblasts, and perichondrial fibroblasts of embryos. Osteonectin modulation of cell adhesion and proliferation may also function in tissue remodeling and angiogenesis (Kupprion et al. (1998) J. Biol. Chem. 45:29635-29640).

FS is associated with a variety of cell proliferative, reproductive, and developmental disorders. Transgenic mice lacking FS have multiple musculoskeletal defects and die shortly after birth (Matzuk, M. M. et al. (1995) Nature 374:360-363). Abnormal expression and localization of FS have been implicated in benign prostatic hyperplasia and prostate cancer (Thomas, T. Z. et al. (1998) Prostate 34:3443). The Follistatin-Related Gene, which encodes a protein with a FS-like polycysteine domain, is associated with chromosomal translocations that may play a role in leukemogenesis (Hayette, S. (1998) Oncogene 16:2949-2954). In the inflammatory response, FS increases the macrophage foam cell formation characteristic of early atherosclerosis (Kozaki, K. et al. (1997) Arterioscler. Thromb. Vasc. Biol. 17:2389-2394).

The bone morphogenetic proteins (BMPs) are bone-derived factors capable of inducing ectopic bone formation (Wozney, J. M. et al. (1988) Science 242:1528-1534). BMPs are hydrophobic glycoproteins involved in bone generation and regeneration, several of which are related to the TGF-beta superfamily. BMP-1, for example, appears to have a regulatory role in bone formation and is characterized by procollagen C-proteinase activity and the presence of an extracellular “CUB” domain. The CUB domain is composed of some 110 residues containing four cysteines which probably form two disulfide bridges, and is found in a variety of functionally diverse, mostly developmentally regulated proteins (ExPASy PROSHIE document PR00908).

Another class of growth factors includes the hematopoietic growth factors, which are narrow in their target specificity. These factors stimulate the proliferation and differentiation of blood cells such as B-lymphocytes, T-lymphocytes, erythrocytes, platelets, eosinophils, basophils, neutrophils, macrophages, and their stem cell precursors. These factors include the colony-stimulating factors (G-CSF, M-CSF, GM-CSF, and CSF1-3), erythropoietin, and the cytokines. The cytokines are specialized hematopoietic factors secreted by cells of the immune system and are discussed in detail below.

Growth factors play critical roles in neoplastic transformation of cells in vitro and in tumor progression in vivo. Overexpression of the large polypeptide growth factors promotes the proliferation and transformation of cells in culture. Inappropriate expression of these growth factors by tumor cells in vivo may contribute to tumor vascularization and metastasis. Inappropriate activity of hematopoietic growth factors can result in anemias, leukemias, and lymphomas. Moreover, growth factors are both structurally and functionally related to oncoproteins, the potentially cancer-causing products of proto-oncogenes. Certain FGF and PDGF family members are themselves homologous to oncoproteins, whereas receptors for some members of the EGF, NGF, and FGF families are encoded by proto-oncogenes. Growth factors also affect the transcriptional regulation of both proto-oncogenes and oncosuppressor genes. (Reviewed in Pimentel, E. (1994) Handbook of Growth Factors, CRC Press, Ann Arbor, Mich.; McKay, I. and I. Leigh, eds. (1993) Growth Factors: A Practical Approach, Oxford University Press, New York, N.Y.; Habenicht, A., ed. (1990) Growth Factors, Differentiation Factors, and Cytokines, Springer-Verlag, New York, N.Y.)

In addition, some of the large polypeptide growth factors play crucial roles in the induction of the primordial germ layers in the developing embryo. This induction ultimately results in the formation of the embryonic mesoderm, ectoderm, and endoderm which in turn provide the framework for the entire adult body plan. Disruption of this inductive process would be catastrophic to embryonic development.

Small Pevtide Factors—Neuropeptides and Vasomediators

Neuropeptides and vasomediators (NP/VM) comprise a family of small peptide factors, typically of 20 amino acids or less. These factors generally function in neuronal excitation and inhibition of vasoconstriction/vasodilation, muscle contraction, and hormonal secretions from the brain and other endocrine tissues. Included in this family are neuropeptides and neuropeptide hormones such as bombesin, neuropeptide Y, neurotensin, neuromedin N, melanocortins, opioids, galanin, somatostatin, tachykinins, urotensin II and related peptides involved in smooth muscle stimulation, vasopressin, vasoactive intestinal peptide, and circulatory system-borne signaling molecules such as angiotensin, complement, calcitonin, endothelins, formyl-methionyl peptides, glucagon, cholecystokinin, gastrin, and many of the peptide hormones discussed above. NP/VMs can transduce signals directly, modulate the activity or release of other neurotransmitters and hormones, and act as catalytic enzymes in signaling cascades. The effects of NP/VMs range from extremely brief to long-lasting. (Reviewed in Martin, C. R. et al. (1985) Endocrine Physiology, Oxford University Press, New York, N.Y., pp. 57-62.)

Cytokines

Cytokines comprise a family of signaling molecules that modulate the immune system and the inflammatory response. Cytokines are usually secreted by leukocytes, or white blood cells, in response to injury or infection. Cytokines function as growth and differentiation factors that act primarily on cells of the immune system such as B- and T-lymphocytes, monocytes, macrophages, and granulocytes. Like other signaling molecules, cytokines bind to specific plasma membrane receptors and trigger intracellular signal transduction pathways which alter gene expression patterns. There is considerable potential for the use of cytokines in the treatment of inflammation and immune system disorders.

Cytokine structure and function have been extensively characterized in vitro. Most cytokines are small polypeptides of about 30 kilodaltons or less. Over 50 cytokines have been identified from human and rodent sources. Examples of cytoline subfamilies include the interferons (IFN-α, -β, and -γ), the interleukins (IL1-IL13), the tumor necrosis factors (TNF-α and -β), and the chemokines. Many cytokines have been produced using recombinant DNA techniques, and the activities of individual cytokines have been determined in vitro. These activities include regulation of leukocyte proliferation, differentiation, and motility.

Cytokines interact with a target through receptors expressed on the surface of the responsive cell. Cytokines bind with hemopoietin receptors, receptor kinases, and tumor necrosis factor (TNF)/nerve growth factor (NGF) receptors by bringing together two receptor subunits. This dimerization of receptor subunits transmits a signal through the plasma membrane to the cell cytoplasm. In the case of protein kinase receptors, such as the receptors for epidermal growth factor (EGF) and insulin, the juxtaposition of the two receptor subunit cytoplasmic domains activates their intrinsic tyrosine kinase activity. As a result, the subunits phosphorylate each other. The resulting phosphorylated tyrosine residues then interact with cytoplasmic proteins containing src homology 2 (SH2) domains. SH2-containing proteins that interact with phosphorylated receptor molecules include phosphatidylinositol 3′-kinase, src kinase family members, GRB2, and shc. These SH2 containing proteins are often associated with other cytoplasmic proteins, such as members of the small, monomeric GTP-binding protein families Ras and Rho, and phosphatases, such as the phosphotyrosine phosphatase SHP-2. The signaling complexes formed by these interactions can initiate signal cascades, such as the kinase cascade involving raf and mitogen activated protein (MAP) kinase, which result in transcriptional regulation and cytoskeleton reorganization. Hemopoietin and TNF/NGF receptors, though they have no intrinsic kinase activity, still activate many of the same signal cascades within responding cells.

Many of the kinases involved in cytokine signaling cascades were first identified as products of oncogenes in cancer cells in which kinase activation was no longer subject to normal cellular controls. In fact, about one third of the known oncogenes encode protein kinases. Furthermore, cellular transformation (oncogenesis) is often accompanied by increased tyrosine phosphorylation activity (Charbonneau, H. and N. K. Tonks (1992) Annu. Rev. Cell Biol. 8:463-493). Thus, the cell must have regulatory systems which keep the cytokine signaling cascades under appropriate control.

Eps8 is a protein which associates with and is phosphorylated by the EGF receptor. Human tumor cell lines contain high constitutive levels of tyrosine-phosphorylated Eps8, and overexpression of Eps8 in NIH3T3 cells expressing the EGF receptor (EGFR) leads to an enhanced mitogenic response and cell overgrowth (Provenzano, C. et al. (1998) Exp. Cell Res. 242:186-200). A family of molecules, which include ABI (Ab1 interactor protein)-1 and ABI-2/e3B1, interact with tyrosine kinases, such as the src-like kinase Ab1, and Eps8. Overexpression of ABI-2/e3B1 in NIH3T3 cells expressing EGFR inhibits the mitogenic response and cell growth. Thus, the ABI family of proteins function as negative regulators of cytokine signaling (Ziemnicka-Kotula, D. et al. (1998) J. Biol. Chem. 273:13681-13692).

The SH2-containing phosphotyrosine phosphatases, SHP-1 and SHP-2, are involved in cytokine signaling. SHP-1, the hemopoietic cell phosphatase, is a potent inhibitor of signaling, whereas SHP-2 is a positive signal transducer for several cytokines. A family of transmembrane glycoproteins, called SIRPs (signal regulatory proteins), are substrates of tyrosine kinases. Phosphorylated SIRPs bind to SHP-2 and have a negative effect on cell response induced by cytokines, including an inhibition of growth factor-induced DNA synthesis. This inhibition correlates with reduced MAP kinase activation in SIRP-transfected NIH3T3 cells stimulated with insulin or EGF. SIRP overexpression also suppressed transformation of NIH3T3 cells by a retrovirus carrying the v-fms oncogene (Kharitonenkov, A. et al. (1997) Nature 386:181-186).

The activity of an individual cytokine in vitro may not reflect the full scope of that cytokine's activity in vivo. Cytokines are not expressed individually in vivo but are instead expressed in combination with a multitude of other cytokines when the organism is challenged with a stimulus. Together, these cytokines collectively modulate the immune response in a manner appropriate for that particular stimulus. Therefore, the physiological activity of a cytokine is determined by the stimulus itself and by complex interactive networks among co-expressed cytokines which may demonstrate both synergistic and antagonistic relationships.

Recently, a unique cytokine has been isolated that appears to have anti-tumor activity in vitro (Ridge, R. J. and N. J. Sloane (1996) Cytokine 8:1-5). This cytokine, anti-neoplastic urinary protein (ANUP), was originally purified as a dimer from human urine. ANUP was later classified as a cytokine when localization studies demonstrated that it was expressed in human granulocytes. ANUP inhibits the growth of cell lines derived from tumors of the breast, skin, lung, bladder, pancreas, and cervix. However, ANUP does not affect the growth of human non-tumor cell lines. The N-terminal 22 amino acids of ANUP comprise a signal peptide which is cleaved from the mature protein. The first nine amino acids of the mature protein retain about 10% of the anti-tumor activity. In addition, ANUP contains a Ly-6/u-PAR sequence motif that is typical of certain cell surface glycoproteins. This motif is characterized by a distinct pattern of six cysteine residues within a 50-residue consensus sequence. The Ly-6/u-PAR motif is found in the Ly-6 T-lymphocyte surface antigen and in the receptor (u-PAR) for urokinase-type plasminogen activator, an extracellular serine protease.

Chemokines comprise a cytokine subfamily with over 30 members. (Reviewed in Wells, T. N. C. and M. C. Peitsch (1997) J. Leukoc. Biol. 61:545-550.) Chemokines were initially identified as chemotactic proteins that recruit monocytes and macrophages to sites of inflammation. Recent evidence indicates that chemokines may also play key roles in hematopoiesis and HIV-1 infection. Chemokines are small proteins which range from about 6-15 kilodaltons in molecular weight. Chemokines are further classified as C, CC, CXC, or CX₃C based on the number and position of certain cysteine residues. The CC chemokines, for example, each contain a conserved motif consisting of two consecutive cysteines followed by two additional cysteines which occur downstream at 24- and 16-residue intervals, respectively (ExPASy PROSITE database, documents PS00472 and PDOC00434). The presence and spacing of these four cysteine residues are highly conserved, whereas the intervening residues diverge significantly. However, a conserved tyrosine located about 15 residues downstream of the cysteine doublet seems to be important for chemotactic activity. Most of the human genes encoding CC chemokines are clustered on chromosome 17, although there are a few examples of CC chemokine genes that map elsewhere. Other chemokines include lymphotactin (C chemokine); macrophage chemotactic and activating factor (MCAF/MCP-1; CC chemokine); platelet factor 4 and IL-8 (CXC chemokines); and fractalkine and neurotractin (CX₃C chemokines). (Reviewed in Luster, A. D. (1998) N. Engl. J. Med. 338:436-445.)

Recently, a novel CC chemokine has been identified in mouse and human thymus (Vicari, A. P. et al. (1997) Immunity 7:291-301). This protein, called thymus-expressed chemokine (TECK), is also expressed at lower levels in the small intestine. TECK likely plays a role in T-lymphocyte development for two reasons. First, TECK is most abundantly expressed in the thymus, which is the major lymphoid organ where T-lymphocyte maturation occurs. Second, the primary source of TECK in the thymus is dendritic cells, which are leukocytic cells that help establish self-tolerance in developing T-lymphocytes. In addition, TECK demonstrates chemotactic activity for activated macrophages, dendritic cells, and thymic T-lymphocytes. The cDNA encoding human TECK (hTECK) contains an open reading frame of 453 base pairs which predicts a protein of 151 amino acids. hTECK retains the conserved features of CC chemokines described above, including four conserved cysteines at C30, C31, C58, and C75. However, the spacing between C31 and C58 is increased by three residues, and the spacing between C58 and C75 is increased by one residue. In addition, hTECK lacks the conserved tyrosine found in most CC chemokines.

Chromogranins and secretogranins are acidic proteins present in the secretory granules of endocrine and neuro-endocrine cells (Huttner, W. B. et al. (1991) Trends Biochem. Sci. 16 27-30) (Simon, J.-P. et al. (1989) Biochem.J. 262 1-13.) Granins may be precursors of biologically-active peptides, or they may be helper proteins in the packaging of peptide hormones and neuropeptides—their precise role is unclear.

Alzheimer's disease (AD) is a progressive dementia characterized neuropathologically by the presence of amyloid β-peptide-containing plaques and neurofibrillary tangles in specific brain regions. In addition, neurons and synapses are lost and inflammatory responses are activated in microglia and astrocytes.

Human Suppressors of Cvtokine Signaling (SOCS) Homologs

Signal transduction is a general process in which cells respond to extracellular signals (hormones, neurotransmitters, growth and differentiation factors, etc.) through a cascade of biochemical reactions beginning with the binding of the signal molecule to a cell membrane receptor and ending with an effect on an intracellular target molecule. Intermediate steps in this process involve the activation of various cytoplasmic proteins by phosphorylation via protein kinases and the translocation of some of these activated proteins to the cell nucleus, where the transcription of specific genes is affected. The signal transduction process regulates all types of cell functions, including cell proliferation, differentiation, and gene transcription.

Many of the cytokine receptors, including those for the growth factors EGF, PDGF, and FGF exhibit intrinsic protein kinase activity. Binding of the cytokine to its receptor triggers the autophosphorylation of a tyrosine residue on the receptor. It is believed that these phosphorylated residues are recognition sites for the binding of other cytoplasmic signaling proteins which link the initial receptor activation at the cell surface to the activation of a specific intracellular target molecule. These signaling proteins contain a src homology 2 (SH2) domain that is a recognition and binding site for the phosphotyrosine residue. SH2 domains are found in a variety of signaling molecules and oncogenic proteins, such as phospholipase C-g, Ras GTP-ase activating protein, and GRB2 (Lowenstein, E. J. et al. (1992) Cell 70:431-442).

While much is known about key events in the activation of signaling pathways, less is known about how they are switched off. Recently, several SH2-containing proteins have been identified that are induced in murine lymphoid cells by various cytokines, including IL-2, IL-3, IL-6, Interferon-γ, and EPO (Yoshimura, A. et al. (1995) EMBO Journal 14:2816-2826; Starr, R. et al. (1997) Nature 387:917-921; and Naka, T. et al. (1997) Nature 387:924-929). A common property of these proteins is the ability to suppress growth and differentiation in murine cells. The induction of these SH2-containing proteins in cytokine stimulated cells suggests that they may function as negative regulators of cytokine signaling. Transcription of the genes encoding four of these proteins, CIS (cytokine-inducible SH2-containing protein), and SOCS-1, -2, and -3 (suppressor of cytokine signaling), is induced by IL-6 both in vitro and in vivo (Starr et al., supra).

The four proteins share little sequence homology in their N-termiinal regions, but all contain a central SH2 domain and a conserved C-terminal region designated the “SOCS box.” The function of the SOCS box is unknown. However, a conserved core triplet sequence (K/R) (D/E) (Y/F) within the SOCS box is similar to the tyrosine phosphorylation site recognized by the JAK kinase family. This similarity suggests that the SOCS box may provide a site for interaction with, and inhibition of, JAK kinases. The finding that SOCS-1 interacts with the catalytic region of JAK kinases supports this hypothesis (Endo, T. A. et al. (1997) Nature 387:921-24). Constitutive expression of SOCS-1 in M1 murine lymphoid cells also inhibits the phosphorylation of certain cell signaling components (gp130 and Stat3) in response to IL-6 (Starr et al., supra). CIS binds to tyrosine-phosphorylated residues in the beta-chain of the IL-3 and EPO receptors and provides another possible mechanism for suppressing cell signaling by preventing the binding of other signaling proteins (Yoshimura et al., supra).

Recently, sixteen additional proteins have been identified containing the SOCS box domain (Hilton, D. J. et al. (1998) Proc. Natl. Acad. Sci. USA 95:114-119). Like the SH2-containing proteins described above, each of the proteins contains a C-terminal SOCS box and a distinctive motif N-terminal of the SOCS box. In addition to four new SOCS proteins containing the SH2 domain, three additional classes of SOCS proteins were found containing WD40 repeats (WSB-1 and -2), SPRY domains (SSB-1 to -3), or ankyrin repeats (ASB-1 to -3). A class of small GTPases (Rar proteins) that contain the SOCS box were also identified. The function of WSB, SSB, and ASB proteins are as yet unknown. However, like SH2 domains, WD-40 repeats, ankyrin repeats, and SPRY domains have been implicated in protein-protein interactions (Hilton et al., supra).

Defects or alterations in the activity of signaling proteins such as CIS may play a role in the development of various proliferative disorders and diseases such as cancer. Loss or rearrangement of the putative human gene encoding CIS is associated with the development of renal cell carcinomas and lung cancer (Yoshimura et al., supra). This association suggests that CIS may function as a tumor suppressor gene.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Culture medium and other growth conditions can influence epithelial cell phenotypes including expression of the cytokeratin markers. In most cases, primary human mammary epithelial cells (HMECs) and immortalized breast cell lines have been grown in monolayer culture on plastic in media containing serum or pituitary extract. The undefined growth factors and hormones contained in serum and pituitary extract can have profound effects on gene expression patterns and cell morphology. Since epithelial cells under physiological conditions are never exposed to serum, these artifact conditions are not ideal for studying the cell biology of normal and malignant cells. MDA-mb-231 is a breast tumor cell line isolated from the pleural effusion of a 51-year old female. It forms poorly differentiated adenocarcinoma in nude mice and ALS treated BALB/c mice. It also expresses the Wnt3 oncogene, EGF, and tumor necrosis factor alpha (TGF-α).

Human aortic endothelial cells (HAECs) are primary cells derived from the endothelium of a human aorta. Human umbilical artery endothelial cells (HUAECs) are primary cells derived from the endothelium of an umbilical artery. HAECs and HUAECs have been used as an experimental model for investigating the role of the endothelium in human vascular biology in vitro. Activation of the vascular endothelium is considered to be a central event in a wide range of both physiological and pathophysiological processes, such as vascular tone regulation, coagulation and thrombosis, atherosclerosis, inflammation, and some infectious diseases.

TNF-α is a pleiotropic cytokine that is known to play a central role in the mediation of inflammatory responses through activation of multiple signal transduction pathways. TNF-α is produced by activated lymphocytes, macrophages, and other white blood cells, and is known to activate endothelial cells.

Lung cancer is the leading cause of cancer death for men and the second leading cause of cancer death for women in the U.S. The vast majority of lung cancer cases are attributed to smoking tobacco, and increased use of tobacco products in third world countries is projected to lead to an epidemic of lung cancer in these countries. Exposure of the bronchial epithelium to tobacco smoke appears to result in changes in tissue morphology, which are thought to be precursors of cancer. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for ˜70% of cases while SCLCs account for ˜18% of cases. The molecular and cellular biology underlying the development and progression of lung cancer are incompletely understood. Deletions on chromosome 3 are common in this disease and are thought to indicate the presence of a tumor suppressor gene in this region. Activating mutations in K-ras are commonly found in lung cancer and are the basis of one of the mouse models for the disease.

Most normal eukaryotic cells, after a certain number of divisions, enter a state of senescence in which cells remain viable and metabolically active but no longer replicate. A number of phenotypic changes such as increased cell size and pH-dependent beta-galactosidase activity, and molecular changes such as the upregulation of particular genes, occur in senescent cells (Shelton (1999) Current Biology 9:939-945). When senescent cells are exposed to mitogens, a number of genes are upregulated, but the cells do not proliferate. Evidence indicates that senescent cells accumulate with age in vivo, contributing to the aging of an organism. In addition, senescence suppresses tumorigenesis, and many genes necessary for senescence also function as tumor suppressor genes, such as p53 and the retinoblastoma susceptibility gene. Most tumors contain cells that have surpassed their replicative limit, i.e. they are immortalized. Many oncogenes immortalize cells as a first step toward tumor formation.

A variety of challenges, such as oxidative stress, radiation, activated oncoproteins, and cell cycle inhibitors, induce a senescent phenotype, indicating that senescence is influenced by a number of proliferative and anti-proliferative signals (Shelton supra). Senescence is correlated with the progressive shortening of telomeres that occurs with each cell division. Expression of the catalytic component of telomerase in cells prevents telomere shortening and imnmortalizes cells such as fibroblasts and epithelial cells, but not other types of cells, such as CD8+ T cells (Migliaccio et al. (2000) J Immmunol 165:4978-4984). Thus, senescence is controlled by telomere shortening as well as other mechanisms depending on the type of cell.

A number of genes that are differentially expressed between senescent and presenescent cells have been identified as part of ongoing studies to understand the role of senescence in aging and tumorigenesis. Most senescent cells are growth arrested in the G1 stage of the cell cycle. While expression of many cell cycle genes is similar in senescent and presenescent cells (Cristofalo (1992) Ann N Y Acad Sci 663:187-194), expression of others genes such as cyclin-dependent kinases p21 and p16, which inhibit proliferation, and cyclins D1 and E is elevated in senescent cells. Other genes that are not directly involved in the cell cycle are also upregulated such as extracellular matrix proteins fibronectin, procollagen, and osteonectin; and proteases such as collagenase, stromelysin, and cathepsin B (Chen (2000) Ann NY Acad Sci 908:111-125). Genes underexpressed in senescent cells include those that encode heat shock proteins, c-fos, and cdc-2 (Chen supra).

The potential application of gene expression profiling is particularly relevant to measuring the toxic response to potential therapeutic compounds and of the metabolic response to therapeutic agents. Diseases treated with steroids and disorders caused by the metabolic response to treatment with steroids include adenomatosis, cholestasis, cirrhosis, hemangioma, Henoch-Scbonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as mifepristone, progesterone, beclomethasone, medroxyprogesterone, budesonide, prednisone, dexamethasone, betamethasone, or danazol with the levels and sequences expressed in normal untreated tissue.

Steroids are a class of lipid-soluble molecules, including cholesterol, bile acids, vitamin D, and hormones, that share a common four-ring structure based on cyclopentanoperhydrophenanthrene and that carrry out a wide variety of functions. Corticosteroids are used to relieve inflammation and to suppress the immune response. They inhibit eosinophil, basophil, and airway epithelial cell function by regulation of cytolines that mediate the inflanmmatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumnin compared with α-fetoprotein iii) conversion of ammonia to urea and glutamine; iv) metabolize aromatic amino acids; and v) proliferate in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416).

Ovarian cancer is the leading cause of death from a gynecologic cancer. The majority of ovarian can-cers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. Genetic variations involved in ovarian cancer development include mutation of p53 and microsatellite instability. Gene expression patterns likely vary when normal ovary is compared to ovarian tumors.

There is a need in the art for new compositions, including nucleic acids and proteins, for the diagnosis, prevention, and treatment of autoimmune/inflammatory disorders, neurological disorders; endocrine disorders; developmental disorders; cell proliferative disorders including cancer; reproductive disorders; cardiovascular disorders; and infections.

### SUMMARY OF THE INVENTION

Various embodiments of the invention provide purified polypeptides, extracellular messengers, referred to collectively as “EXMES” and individually as “EXMES-1,” “EXMES-2,” “EXMES-3,” “EXMES-4,” “EXMES-5,” “EXMES-6,” “EXMES-7,” “EXMES-8,” “EXMES-9,” “EXMES-10,” “EXMES-11,” “EXMES-12,” “EXMES-13,” “EXMES-14,” “EXMES-15,” “EXMES-16,” “EXMES-17,” “EXMES-18,” “EXMES-19,” “EXMES-20,” “EXMES-21,” and “EXMES-22,” and methods for using these proteins and their encoding polynucleotides for the detection, diagnosis, and treatment of diseases and medical conditions. Embodiments also provide methods for utilizing the purified extracellular messengers and/or their encoding polynucleotides for facilitating the drug discovery process, including determination of efficacy, dosage, toxicity, and pharmacology. Related embodiments provide methods for utilizing the purified extracellular messengers and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO: 1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. Another embodiment provides an isolated polypeptide comprising an amino acid sequence of SEQ ID NO:1-22.

Still another embodiment provides an isolated polynucleotide encoding a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. In another embodiment, the polynucleotide encodes a polypeptide selected from the group consisting of SEQ ID NO:1-22. In an alternative embodiment, the polynucleotide is selected from the group consisting of SEQ ID NO:23-44.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22.

Still yet another embodiment provides an isolated polynucleotide selected from the group consisting of a) a polynucleotide comprising a polynucleotide sequence selected from the group consisting of SEQ ID NO:23-44, b) a polynucleotide comprising a naturally occurring polynucleotide sequence at least 90% identical or at least about 90% identical to a polynucleotide sequence selected from the group consisting of SEQ ID NO:23-44, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:23-44, b) a polynucleotide comprising a naturally occurring polynucleotide sequence at least 90% identical or at least about 90% identical to a polynucleotide sequence selected from the group consisting of SEQ ID NO:23-44, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:23-44, b) a polynucleotide comprising a naturally occurring polynucleotide sequence at least 90% identical or at least about 90% identical to a polynucleotide sequence selected from the group consisting of SEQ ID NO:23-44, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22, and a pharmaceutically acceptable excipient. In one embodiment, the composition can comprise an amino acid sequence selected from the group consisting of SEQ ID NO:1-22. Other embodiments provide a method of treating a disease or condition associated with decreased or abnormal expression of functional EXMES, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional EXMES, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional EXMES, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-22, b) a polypeptide comprising a naturally occurring amino acid sequence at least 90% identical or at least about 90% identical to an amino acid sequence selected from the group consisting of SEQ ID NO:1-22, c) a biologically active fragment of a polypeptide having an amino acid sequence selected from the group consisting of SEQ ID NO:1-22, and d) an immunogenic fragment of a polypeptide having an amino acid sequence selected from the group consisting of SEQ ID NO:1-22. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:23-44, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:23-44, ii) a polynucleotide comprising a naturally occurring polynucleotide sequence at least 90% identical or at least about 90% identical to a polynucleotide sequence selected from the group consisting of SEQ ID NO:23-44, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:23-44, ii) a polynucleotide comprising a naturally occurring polynucleotide sequence at least 90% identical or at least about 90% identical to a polynucleotide sequence selected from the group consisting of SEQ ID NO:23-44, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide embodiments, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“EXMES” refers to the amino acid sequences of substantially purified EXMES obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of EXMES. Agonists may include proteins, nucleic acids, carbohydrates, small molecules, or any other compound or composition which modulates the activity of EXMES either by directly interacting with EXMES or by acting on components of the biological pathway in which EXMES participates.

An “allelic variant” is an alternative form of the gene encoding EXMES. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding EXMES include those sequences with deletions, insertions, or substitutions of different nucleotides, resulting in a polypeptide the same as EXMES or a polypeptide with at least one functional characteristic of EXMES. Included within this definition are polymorphisms which may or may not be readily detectable using a particular oligonucleotide probe of the polynucleotide encoding EXMES, and improper or unexpected hybridization to allelic variants, with a locus other than the normal chromosomal locus for the polynucleotide encoding EXMES. The encoded protein may also be “altered,” and may contain deletions, insertions, or substitutions of amino acid residues which produce a silent change and result in a functionally equivalent EXMES. Deliberate amino acid substitutions may be made on the basis of one or more similarities in polarity, charge, solubility, hydrophobicity, hydrophilicity, and/or the amphipathic nature of the residues, as long as the biological or immunological activity of EXMES is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of EXMES. Antagonists may include proteins such as antibodies, anticalins, nucleic acids, carbohydrates, small molecules, or any other compound or composition which modulates the activity of EXMES either by directly interacting with EXMES or by acting on components of the biological pathway in which EXMES participates.

The term “antibody” refers to intact immunoglobulin molecules as well as to fragments thereof, such as Fab, F(ab′)₂, and Fv fragments, which are capable of binding an epitopic determinant. Antibodies that bind EXMES polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic EXMES, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding EXMES or fragments of EXMES may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of EXMES or a polynucleotide encoding EXMES which can be identical in sequence to, but shorter in length than, the parent sequence. A fragment niay comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:23-44 can comprise a region of unique polynucleotide sequence that specifically identifies SEQ ID NO:23-44, for example, as distinct from any other sequence in the genome from which the fragment was obtained. A fragment of SEQ ID NO:23-44 can be employed in one or more embodiments of methods of the invention, for example, in hybridization and amplification technologies and in analogous methods that distinguish SEQ ID NO:23-44 from related polynucleotides. The precise length of a fragment of SEQ ID NO:23-44 and the region of SEQ ID NO:23-44 to which the fragment corresponds are routinely determinable by one of ordinary skill in the art based on the intended purpose for the fragment.

A fragment of SEQ ID NO:1-22 is encoded by a fragment of SEQ ID NO:23-44. A fragment of SEQ ID NO:1-22 can comprise a region of unique amino acid sequence that specifically identifies SEQ ID NO:1-22. For example, a fragment of SEQ ID NO:1-22 can be used as an immunogenic peptide for the development of antibodies that specifically recognize SEQ ID NO:1-22. The precise length of a fragment of SEQ ID NO:1-22 and the region of SEQ ID NO:1-22 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved” =4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr.-21-2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=l, gap penalty=3, window=5, and “diagonals saved” =5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr.-21-2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tm is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of EXMES which is capable of eliciting an immune response when introduced into a living organism, for example, a mammal. The term “immunogenic fragrnent” also includes any polypeptide or oligopeptide fragment of EXMES which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of EXMES. For example, modulation may cause an increase or a decrease in protein activity, binding characteristics, or any other biological, functional, or immunological properties of EXMES.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an EXMES may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of EXMES.

“Probe” refers to nucleic acids encoding EXMES, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing EXMES, nucleic acids encoding EXMES, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

Various embodiments of the invention include new human extracellular messengers (EXMES), the polynucleotides encoding EXMES, and the use of these compositions for the diagnosis, treatment, or prevention of autoimmune/inflammatory disorders, neurological disorders; endocrine disorders; developmental disorders; cell proliferative disorders including cancer; reproductive disorders; cardiovascular disorders; and infections.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to polypeptide and polynucleotide embodiments. The full length clones encode polypeptides which have at least 95% sequence identity to the polypeptides shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are extracellular messengers. For example, SEQ ID NO:1 is 100% identical, from residue M15 to residue G725, to human hepatocyte growth factor-like protein (GenBank ID g1311661) as determined by the Basic Local Alignment Search Tool (BLAST). (See Table 2.) The BLAST probability score is 0.0, which indicates the probability of obtaining the observed polypeptide sequence alignment by chance. SEQ ID NO:1 also contains Pan, kringle, and trypsin-like domains, which are found in hepatocyte growth factor, as determined by searching for statistically significant matches in the hidden Markov model (HMM)-based PFAM database of conserved protein family domains. (See Table 3.) Data from BLIMPS, MOTIFS, and PROFILESCAN analyses and BLAST analyses of the PRODOM and DOMO databases provide further corroborative evidence that SEQ ID NO:1 is a growth factor. In another example, SEQ ID NO:3 is 96% identical, from residue V37 to residue E350, to human transforming growth factor-beta 1 binding protein precursor (GenBank ID g339548) as determined by BLAST. The BLAST probability score is 3.8e-178. SEQ ID NO:3 also contains EGF-like domains and a TB domain as determined by searching for statistically significant matches in the hidden Markov model (HMM)-based PFAM database. Data from BLIMPS, MOTIFS, and further BLAST analyses provide corroborative evidence that SEQ ID NO:3 is a human transforming growth factor-beta 1 binding protein precursor. In another example, SEQ ID NO:7 is 93% identical, from residue C650 to residue E1668, to human transforming growth factor-beta 1 binding protein precursor (GenBank ID g339548) as determined by BLAST. The BLAST probability score is 0.0. SEQ ID NO:7 also contains an EGF-like domain and a TB domain as determined by searching for statistically significant matches in the hidden Markov model (HMM)-based PFAM database. Data from BLIMPS, MOTIFS, and further BLAST analyses provide corroborative evidence that SEQ ID NO:7 is a transforming growth factor-beta 1 binding protein precursor. In a further example, SEQ ID NO:14 is 96% identical, from residue MI to residue Q958, to human transforming growth factor-beta 1 binding protein precursor (GenBank ID g339548) as determined by BLAST. The BLAST probability score is 0.0. SEQ ID NO:14 is expressed in tissues which express TGF-beta 1, is involved in assembly and secretion of latent TGF-beta, and is a latent TGF-beta binding protein, as determined by BLAST analysis using the PROTEOME database. SEQ ID NO:14 also contains a EGF-like domain and a TB domain as determined by searching for statistically significant matches in the hidden Markov model (HMM)-based PFAM database. Data from BLIMPS, MOTIFS, and further BLAST analyses provide corroborative evidence that SEQ ID NO:14 is a human transforming growth factor-beta 1 binding protein precursor. In yet another example, SEQ ID NO:18 is 100% identical, from residue K9 to residue N104, to human prolactin (GenBank ID g531103) as determined by BLAST. The BLAST probability score is 6.6e-82. SEQ ID NO:18 also has homology to prolactin and placental lactogen II, as determined by BLAST analysis using the PROTEOME database. SEQ ID NO:18 also contains a somatotropin hormone family domain as determined by searching for statistically significant matches in the hidden Markov model (HMM)-based PFAM database. Data from BLIMPS, MOTIFS, and PROFILESCAN analyses provide further corroborative evidence that SEQ ID NO:18 is a prolactin. In another example, SEQ ID NO:22 is 99% identical, from residue M1 to residue L165, to H. sapiens reading frame prolactin (GenBank ID g3421 1) as determined by BLAST. The BLAST probability score is 3.2e-83. SEQ ID NO:22 also has homology to proteins that are localized to the extracellular region, have roles in angiogenesis inhibition,and control of cell proliferation, and have homology to human and rat prolactin, as determined by BLAST analysis using the PROTEOME database. SEQ ID NO:22 also contains a somatotropin hormone family domain as determined by searching for statistically significant matches in the hidden Markov model (HMM)-based PFAM database. Data from BLIMPS, MOTIFS, PROFILESCAN and additional BLAST analyses of the DOMO and PRODOM databases provide further corroborative evidence that SEQ ID NO:22 is a member of the somatotropin hormone family. SEQ ID NO:2, SEQ ID NO:4-6, SEQ ID NO:8-13, SEQ ID NO:15-17, and SEQ ID NO:19-21 were analyzed and annotated in a similar manner. The algorithms and parameters for the analysis of SEQ ID NO:1-22 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genornic sequences used to assemble the full length polynucleotide embodiments, and of fragments of the polynucleotides which are useful, for example, in hybridization or amplification technologies that identify SEQ ID NO:23-44 or that distinguish between SEQ ID NO:23-44 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm For example, a polynucleotide sequence identified as FL_XXXXXX_N₁_{_{—}}N₂_{_{—}}YYYYY_N₃_{_{—}}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1₁₃N is a “stretched” sequence, with XXXXY being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, Cambridge, |
| | UK). |
| GBI | Hand-edited analysis of genomic sequences. |
| FL | Stitched or stretched genomic sequences (see Example V). |
| INCY | Full length transcript and exon prediction from mapping of EST |
| | sequences to the genome. Genomic location and EST |
| | composition data are combined to predict the exons |
| | and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide embodiments, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ED), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses EXMES variants. A preferred EXMES variant is one which has at least about 80%, or alternatively at least about 90%, or even at least about 95% amino acid sequence identity to the EXMES amino acid sequence, and which contains at least one functional or structural characteristic of EXMES.

Various embodiments also encompass polynucleotides which encode EXMES. In a particular embodiment, the invention encompasses a polynucleotide sequence comprising a sequence selected from the group consisting of SEQ ID NO:23-44, which encodes EXMES. The polynucleotide sequences of SEQ ID NO:23-44, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding EXMES. In particular, such a variant polynucleotide will have at least about 70%, or alternatively at least about 85%, or even at least about 95% polynucleotide sequence identity to a polynucleotide encoding EXMES. A particular aspect of the invention encompasses a variant of a polynucleotide comprising a sequence selected from the group consisting of SEQ ID NO:23-44 which has at least about 70%, or alternatively at least about 85%, or even at least about 95% polynucleotide sequence identity to a nucleic acid sequence selected from the group consisting of SEQ ID NO:23-44. Any one of the polynucleotide variants described above can encode a polypeptide which contains at least one functional or structural characteristic of EXMES.

In addition, or in the alternative, a polynucleotide variant of the invention is a splice variant of a polynucleotide encoding EXMES. A splice variant may have portions which have significant sequence identity to a polynucleotide encoding EXMES, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding EXMES over its entire length; however, portions of the splice variant will have at least about 70%, or alternatively at least about 85%, or alternatively at least about 95%, or alternatively 100% polynucleotide sequence identity to portions of the polynucleotide encoding EXMES. For example, a polynucleotide comprising a sequence of SEQ ID NO:40, a polynucleotide comprising a sequence of SEQ ID NO:43, and a polynucleotide comprising a sequence of SEQ ID NO:44 are splice variants of each other. In another example, a polynucleotide comprising a sequence of SEQ ID NO:26, and a polynucleotide comprising a sequence of SEQ ID NO:30 are splice variants of each other. In a further example, a polynucleotide comprising a sequence of SEQ ID NO:32, a polynucleotide comprising a sequence of SEQ ID NO:33, and a polynucleotide comprising a sequence of SEQ ID NO:34 are splice variants of each other. In yet a further example, a polynucleotide comprising a sequence of SEQ ID NO:35, a polynucleotide comprising a sequence of SEQ ID NO:36, and a polynucleotide comprising a sequence of SEQ ID NO:37 are splice variants of each other. Any one of the splice variants described above can encode a polypeptide which contains at least one functional or structural characteristic of EXMES.

It will be appreciated by those skilled in the art that as a result of the degeneracy of the genetic code, a multitude of polynucleotide sequences encoding EXMES, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring EXMES, and all such variations are to be considered as being specifically disclosed.

Although polynucleotides which encode EXMES and its variants are generally capable of hybridizing to polynucleotides encoding naturally occurring EXMES under appropriately selected conditions of stringency, it may be advantageous to produce polynucleotides encoding EXMES or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding EXMES and its derivatives without altering the encoded amino acid sequences include the production of RNA transcripts having more desirable properties, such as a greater half-life, than transcripts produced from the naturally occurring sequence.

The invention also encompasses production of polynucleotides which encode EXMES and EXMES derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding EXMES or any fragment thereof.

Embodiments of the invention can also include polynucleotides that are capable of hybridizing to the claimed polynucleotides, and, in particular, to those having the sequences shown in SEQ ID NO:23-44 and fragments thereof, under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acids encoding EXMES may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confrrm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode EXMES may be cloned in recombinant DNA molecules that direct expression of EXMES, or fragments or functional equivalents thereof, in appropriate host cells. Due to the inherent degeneracy of the genetic code, other polynucleotides which encode substantially the same or a functionally equivalent polypeptides may be produced and used to express EXMES.

The polynucleotides of the invention can be engineered using methods generally known in the art in order to alter EXMES-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of EXMES, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding EXMES may be synthesized, in whole or in part, using one or more chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, EXMES itself or a fragment thereof may be synthesized using chemical methods known in the art. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of EXMES, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active EXMES, the polynucleotides encoding EXMES or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding EXMES. Such elements may vary in their strength and specificity. Specific initiation signals may also be used to achieve more efficient translation of pplynucleotides encoding EXMES. Such signals include the ATG initiation codon and adjacent sequences, e.g. the Kozak sequence. In cases where a polynucleotide sequence encoding EXMES and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing polynucleotides encoding EXMES and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express polynucleotides encoding EXMES. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of polynucleotides to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotides encoding EXMES. For example, routine cloning, subcloning, and propagation of polynucleotides encoding EXMES can be achieved using a multifunctional E. coli vector such as PBLUESCRIPT (Stratagene, La Jolla Calif.) or PSPORT1 plasrnid (Invitrogen). Ligation of polynucleotides encoding EXMES into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of EXMES are needed, e.g. for the production of antibodies, vectors which direct high level expression of EXMES may be used. For example, vectors containing the strong, inducible SP6 or T7 bacteriophage promoter may be used.

Yeast expression systems may be used for production of EXMES. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of EXMES. Transcription of polynucleotides encoding EXMES may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, polynucleotides encoding EXMES may be ligated into an adenovirus transcription/translation complex consisting of the late promoter and tripartite leader sequence. Insertion in a non-essential E1 or E3 region of the viral genome may be used to obtain infective virus which expresses EXMES in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of EXMES in cell lines is preferred. For example, polynucleotides encoding EXMES can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S.C. and R.C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding EXMES is inserted within a marker gene sequence, transformed cells containing polynucleotides encoding EXMES can be identified by the absence of marker gene function. Alternatively, a marker gene can be placed in tandem with a sequence encoding EXMES under the control of a single promoter. Expression of the marker gene in response to induction or selection usually indicates expression of the tandem gene as well.

In general, host cells that contain the polynucleotide encoding EXMES and that express EXMES may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of EXMES using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on EXMES is preferred, but a competitive binding assay may be employed. These and other assays are well known in the art. (See, e.g., Hampton, R. et al. (1990) Serological Methods. a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding EXMES include oligolabeling, nick translation, end-labeling, or PCR amplification using a labeled nucleotide. Alternatively, polynucleotides encoding EXMES, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding EXMES may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode EXMES may be designed to contain signal sequences which direct secretion of EXMES through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding EXMES may be ligated to a heterologous sequence resulting in translation of a fusion protein in any of the aforementioned host systems. For example, a chimeric EXES protein containing a heterologous moiety that can be recognized by a commercially available antibody may facilitate the screening of peptide libraries for inhibitors of EXMES activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffmity purification of fusion proteins using commercially available monoclonal and polyclonal antibodies that specifically recognize these epitope tags. A fusion protein may also be engineered to contain a proteolytic cleavage site located between the EXMES encoding sequence and the heterologous protein sequence, so that EXMES may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In another embodiment, synthesis of radiolabeled EXMES may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

EXMES, fragmnents of EXMES, or variants of EXMES may be used to screen for compounds that specifically bind to EXMES. One or more test compounds may be screened for specific binding to EXMES. In various embodiments, 1, 2, 3,4, 5, 10, 20, 50, 100, or 200 test compounds can be screened for specific binding to EXMES. Examples of test compounds can include antibodies, anticalins, oligonucleotides, proteins (e.g., ligands or receptors), or small molecules.

In related embodiments, variants of EXMES can be used to screen for binding of test compounds, such as antibodies, to EXMES, a variant of EXMES, or a combination of EXMES and/or one or more variants EXMES. In an embodiment, a variant of EXMES can be used to screen for compounds that bind to a variant of EXMES, but not to EXMES having the exact sequence of a sequence of SEQ ID NO:1-22. EXMES variants used to perform such screening can have a range of about 50% to about 99% sequence identity to EXMES, with various embodiments having 60%, 70%, 75%, 80%, 85%, 90%, and 95% sequence identity.

In an embodiment, a compound identified in a screen for specific binding to EXMES can be closely related to the natural ligand of EXMES, e.g., a ligand or fragment thereof, a natural substrate, a structural or functional mimetic, or a natural binding partner. (See, e.g., Coligan, J. E. et al. (1991) Current Protocols in Immunology 1(2):Chapter 5.) In another embodiment, the compound thus identified can be a natural ligand of a receptor EXMES. (See, e.g., Howard, A. D. et al. (2001) Trends Pharmacol. Sci.22:132-140; Wise, A. et al. (2002) Drug Discovery Today 7:235-246.)

In other embodiments, a compound identified in a screen for specific binding to EXMES can be closely related to the natural receptor to which EXMES binds, at least a fragment of the receptor, or a fragment of the receptor including all or a portion of the ligand binding site or binding pocket. For example, the compound may be a receptor for EXMES which is capable of propagating a signal, or a decoy receptor for EXMES which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; inunex Corp., Seattle Wash.), which is efficacious for treating rheumatoid arthritis in humans. Etanercept is an engineered p75 tumor necrosis factor (TNF) receptor dimer linked to the Pc portion of human IgG₁ (Taylor, P. C. et al. (2001) Curr. Opin. Immunol. 13:611-616).

In one embodiment, two or more antibodies having similar or, alternatively, different specificities can be screened for specific binding to EXMES, fragments of EXMES, or variants of EXMES. The binding specificity of the antibodies thus screened can thereby be selected to identify particular fragments or variants of EXMES. In one embodiment, an antibody can be selected such that its binding specificity allows for preferential identification of specific fragments or variants of EXMES. In another embodiment, an antibody can be selected such that its binding specificity allows for preferential diagnosis of a specific disease or condition having increased, decreased, or otherwise abnormal production of EXMES.

In an embodiment, anticalins can be screened for specific binding to EXMES, fragments of EXMES, or variants of EXMES. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit EXMES involves producing appropriate cells which express EXMES, either as a secreted protein or on the cell membrane. Preferred cells include cells from mammals, yeast, Drosophila, or E. coli. Cells expressing EXMES or cell membrane fractions which contain EXMES are then contacted with a test compound and binding, stimulation, or inhibition of activity of either EXMES or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with EXMES, either in solution or affixed to a solid support, and detecting the binding of EXMES to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. Nos. 5,914,236 and 6,372,724. In a related embodiment, one or more amino acid substitutions can be introduced into a polypeptide compound (such as a receptor) to improve or alter its ability to bind to its natural ligands. (See, e.g., Matthews, D. J. and J. A. Wells. (1994) Chem. Biol. 1:25-30.) In another related embodiment, one or more amino acid substitutions can be introduced into a polypeptide compound (such as a ligand) to improve or alter its ability to bind to its natural receptors. (See, e.g., Cunningham, B. C. and J. A. Wells (1991) Proc. Natl. Acad. Sci. USA 88:3407-3411; Lowman, H. B. et al. (1991) J. Biol. Chem. 266:10982-10988.)

EXMES, fragments of EXMES, or variants of EXMES may be used to screen for compounds that modulate the activity of EXMES. Such compounds may include agonists, antagonists, or partial or inverse agonists. In one embodiment, an assay is performed under conditions permissive for EXMES activity, wherein EXMES is combined with at least one test compound, and the activity of EXMES in the presence of a test compound is compared with the activity of EXMES in the absence of the test compound. A change in the activity of EXMES in the presence of the test compound is indicative of a compound that modulates the activity of EXMES. Alternatively, a test compound is combined with an in vitro or cell-free system comprising EXMES under conditions suitable for EXMES activity, and the assay is performed. In either of these assays, a test compound which modulates the activity of EXMES may do so indirectly and need not come in direct contact with the test compound. At least one and up to a plurality of test compounds may be screened.

In another embodiment, polynucleotides encoding EXMES or their mammalian homologs may be “knocked out” in an animal model system using homologous recombination in embryonic stem (ES) cells. Such techniques are well known in the art and are useful for the generation of animal models of human disease. (See, e.g., U.S. Pat. Nos. 5,175,383 and 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding EXMES may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding EXMES can also be used to create “knockin” humanized animals (pigs) or transgenic animals (mice or rats) to model human disease. With knockin technology, a region of a polynucleotide encoding EXMES is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a manual inbred to overexpress EXMES, e.g., by secreting EXMES in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of EXMES and extracellular messengers. In addition, examples of tissues expressing EXMES can be found in Table 6 and can also be found in Example XI. Therefore, EXMES appears to play a role in autoimmune/inflammatory disorders, neurological disorders; endocrine disorders; developmental disorders; cell proliferative disorders including cancer; reproductive disorders; cardiovascular disorders; and infections. In the treatment of disorders associated with increased EXMES expression or activity, it is desirable to decrease the expression or activity of EXMES. In the treatment of disorders associated with decreased EXMES expression or activity, it is desirable to increase the expression or activity of EXMES.

Therefore, in one embodiment, EXMES or a fragment or derivative thereof may be administered to a subject to treat or prevent a disorder associated with decreased expression or activity of EXMES. Examples of such disorders include, but are not limited to, an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma; a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism; a disorder associated with hyperpituitarism including acromegaly, giantism, and syndrome of inappropriate antidiuretic hormone (ADH) secretion (SIADH) often caused by benign adenoma; a disorder associated with hypothyroidism including goiter, myxedema, acute thyroiditis associated with bacterial infection, subacute thyroiditis associated with viral infection, autoimmune thyroiditis (Hashimoto's disease), and cretinism; a disorder associated with hyperthyroidism including thyrotoxicosis and its various forms, Grave's disease, pretibial myxedema, toxic multinodular goiter, thyroid carcinoma, and Plummer's disease; a disorder associated with hyperparathyroidism including Conn disease (chronic hypercalemia); a pancreatic disorder such as Type I or Type II diabetes mellitus and associated complications; a disorder associated with the adrenals such as hyperplasia, carcinoma, or adenoma of the adrenal cortex, hypertension associated with alalosis, amyloidosis, hypokalemia, Cushing's disease, Liddle's syndrome, and Arnold-Healy-Gordon syndrome, pheochromocytoma tumors, and Addison's disease; a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, a hypergonadal disorder associated with Leydig cell tumors, androgen resistance associated with absence of androgen receptors, syndrome of 5 a-reductase, and gynecomastia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcmiioma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a reproductive disorder, such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, benign prostatic hyperplasia, prostatitis, Peyronie's disease, and impotence; a cardiovascular disorder, such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; and an infection such as that caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picomavirus, poxvirus, reovirus, retrovirus, rhabdovirus, or togavirus; an infection such as that caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, and campylobacter, pseudomonas, vibrio, brucella, francisella, yersinia, bartonella, norcardium, actinomyces, mycobacterium, spirochaetale, rickettsia, chlamydia, or mycoplasma; an infection such as that caused by a fungal agent classified as aspergillus, blastomyces, dermatophytes, cryptococcus, coccidioides, malasezzia, histoplasma, or other fungal agents causing various mycoses; and an infection such as that caused by a parasite classified as plasmodium or malaria-causing, parasitic entamoeba, leishmania, trypanosorna, toxoplasrna, pneumocystis carinii, intestinal protozoa such as giardia, trichomonas, tissue nematodes such as trichinella, intestinal nematodes such as ascaris, lymphatic filarial nematodes, trematodes such as schistosoma, or cestrodes such as tapeworm.

In another embodiment, a vector capable of expressing EXMES or a fragment or derivative thereof may be administered to a subject to treat or prevent a disorder associated with decreased expression or activity of EXMES including, but not limited to, those described above.

In a further embodiment, a composition comprising a substantially purified EXMES in conjunction with a suitable pharmaceutical carrier may be administered to a subject to treat or prevent a disorder associated with decreased expression or activity of EXMES including, but not limited to, those provided above.

In still another embodiment, an agonist which modulates the activity of EXMES may be administered to a subject to treat or prevent a disorder associated with decreased expression or activity of EXMES including, but not limited to, those listed above.

In a further embodiment, an antagonist of EXMES may be administered to a subject to treat or prevent a disorder associated with increased expression or activity of EXMES. Examples of such disorders include, but are not limited to, those autoimmune/inflanmmatory disorders, neurological disorders; endocrine disorders; developmental disorders; cell proliferative disorders including cancer; reproductive disorders; cardiovascular disorders; and infections described above. In one aspect, an antibody which specifically binds EXMES may be used directly as an antagonist or indirectly as a targeting or delivery mechanism for bringing a pharmaceutical agent to cells or tissues which express EXMES.

In an additional embodiment, a vector expressing the complement of the polynucleotide encoding EXMES may be administered to a subject to treat or prevent a disorder associated with increased expression or activity of EXMES including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of EXMES may be produced using methods which are generally known in the art. In particular, purified EXMES may be used to produce antibodies or to screen libraries of pharmaceutical agents to identify those which specifically bind EXMES. Antibodies to EXMES may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with EXMES or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to EXMES have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of EXMES amino acids may be fused with those of another protein, such as KLH, and antibodies to the chimeric molecule may be produced.

Monoclonal antibodies to EXMES may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce EXMES-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for EXMES may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between EXMES and its specific antibody. A two-site, monoclonal-based immunoassay utilizing monoclonal antibodies reactive to two non-interfering EXMES epitopes is generally used, but a competitive binding assay may also be employed (Pound, supra).

Various methods such as Scatchard analysis in conjunction with radioimmunoassay techniques may be used to assess the affinity of antibodies for EXMES. Affinity is expressed as an association constant, Kₐ, which is defined as the molar concentration of EXMES-antibody complex divided by the molar concentrations of free antigen and free antibody under equilibrium conditions. The Kₐ determined for a preparation of polyclonal antibodies, which are heterogeneous in their affinities for multiple EXMES epitopes, represents the average affinity, or avidity, of the antibodies for EXMES. The Kₐ determined for a preparation of monoclonal antibodies, which are monospecific for a particular EXMES epitope, represents a true measure of affinity. High-affmity antibody preparations with Kₐ ranging from about 10⁹ to 10¹² L/mole are preferred for use in immunoassays in which the EXMES-antibody complex must withstand rigorous manipulations. Low-affmity antibody preparations with Kₐ ranging from about 10⁶ to 10⁷ L/mole are preferred for use in immunopurification and similar procedures which ultimately require dissociation of EXMES, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of EXMES-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, polynucleotides encoding EXMES, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding EXMES. Such technology is well known in the art, and antisense oligonucleotides or larger fragments can be designed from various locations along the coding or control regions of sequences encoding EXMES. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding EXMES may be used for somatic or germline gene therapy. Gene therapy may be performed to (i) correct a genetic deficiency (e.g., in the cases of severe combined imrnmunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoina cruzi). In the case where a genetic deficiency in EXMES expression or regulation causes disease, the expression of EXMES from an appropriate population of transduced cells may alleviate the clinical manifestations caused by the genetic deficiency.

In a further embodiment of the invention, diseases or disorders caused by deficiencies in EXMES are treated by constructing mammalian expression vectors encoding EXMES and introducing these vectors by mechanical means into EXMES-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Reécipon (1998) Curr. Opin. Biotechnol. 9:445-450).

Expression vectors that may be effective for the expression of EXMES include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). EXMES may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding EXMES from a normal individual.

Commercially available liposome transformation kits (e.g., the PERFECT LIPED TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to EXMES expression are treated by constructing a retrovirus vector consisting of (i) the polynucleotide encoding EXMES under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding EXMES to cells which have one or more genetic abnormalities with respect to the expression of EXMES. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another embodiment, a herpes-based, gene therapy delivery system is used to deliver polynucleotides encoding EXMES to target cells which have one or more genetic abnormalities with respect to the expression of EXMES. The use of herpes simplex virus (HSV)-based vectors may be especially valuable for introducing EXMES to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res, 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding EXMES to target cells. The biology of the prototypic alphavirus, Serniki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for EXMES into the alphavirus genome in place of the capsid-coding region results in the production of a large number of EXMES-coding RNAs and the synthesis of high levels of EXMES in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of EXMES into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding EXMES.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding EXMES. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding EXMES. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased EXMES expression or activity, a compound which specifically inhibits expression of the polynucleotide encoding EXMES may be therapeutically useful, and in the treatment of disorders associated with decreased EXMES expression or activity, a compound which specifically promotes expression of the polynucleotide encoding EXMES may be therapeutically useful.

At least one, and up to a plurality, of test comipounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding EXMES is exposed to at least one test compound thus obtained. The sample may comprise, for example, an intact or permeabilized cell, or an int vitro cell-free or reconstituted biochemical system. Alterations in the expression of a polynucleotide encoding EXMES are assayed by any method commonly known in the art. Typically, the expression of a specific nucleotide is detected by hybridization with a probe having a nucleotide sequence complementary to the sequence of the polynucleotide encoding EXMES. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of EXMES, antibodies to EXMES, and mimetics, agonists, antagonists, or inhibitors of EXMES.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising EXMES or fragments thereof. For example, liposome preparations containing a cell-impermeable macromolecule may promote cell fusion and intracellular delivery of the macromolecule. Alternatively, EXMES or a fragment thereof may be joined to a short cationic N-terminal portion from the HUV Tat-1 protein. Fusion proteins thus generated have been found to transduce into the cells of all tissues, including the brain, in a mouse niodel system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example EXMES or fragments thereof, antibodies of EXMES, and agonists, antagonists or inhibitors of EXMES, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and. administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 ,μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind EXMES may be used for the diagnosis of disorders characterized by expression of EXMES, or in assays to monitor patients being treated with EXMES or agonists, antagonists, or inhibitors of EXMES. Antibodies useful for diagnostic purposes may be prepared in the same manner as described above for therapeutics. Diagnostic assays for EXMES include methods which utilize the antibody and a label to detect EXMES in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring EXMES, including ELISAs, RIAs, and FACS, are known in the art and provide a basis for diagnosing altered or abnormal levels of EXMES expression. Normal or standard values for EXMES expression are established by combining body fluids or cell extracts taken from normal mammalian subjects, for example, human subjects, with antibodies to EXMES under conditions suitable for complex formation. The amount of standard complex formation may be quantitated by various methods, such as. photometric means. Quantities of EXMES expressed in subject, control, and disease samples from biopsied tissues are compared with the standard values. Deviation between standard and subject values establishes the parameters for diagnosing disease.

In another embodiment of the invention, polynucleotides encoding EXMES may be used for diagnostic purposes. The polynucleotides which may be used include oligonucleotides, complementary RNA and DNA molecules, and PNAs. The polynucleotides may be used to detect and quantify gene expression in biopsied tissues in which expression of EXMES may be correlated with disease. The diagnostic assay may be used to determine absence, presence, and excess expression of EXMES, and to monitor regulation of EXMES levels during therapeutic intervention.

In one aspect, hybridization with PCR probes which are capable of detecting polynucleotides, including genomic sequences, encoding EXMES or closely related molecules may be used to identify nucleic acid sequences which encode EXMES. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding EXMES, allelic variants, or related sequences.

Probes may also be used for the detection of related sequences, and may have at least 50% sequence identity to any of the EXMES encoding sequences. The hybridization probes of the subject invention may be DNA or RNA and may be derived from the sequence of SEQ ID NO:23-44 or from genomic sequences including promoters, enhancers, and introns of the EXMES gene.

Means for producing specific hybridization probes for polynucleotides encoding EXMES include the cloning of polynucleotides encoding EXMES or EXMES derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or 35S, or by enzymatic labels, such as alkaline phosphatase coupled to the probe via avidin/biotin coupling systems, and the like.

Polynucleotides encoding EXMES may be used for the diagnosis of disorders associated with expression of EXMES. Examples of such disorders include, but are not limited to, an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helrninthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders; progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an endocrine disorder such as a disorder of the hypothalamus and/or pituitary resulting from lesions such as a primary brain tumor, adenoma, infarction associated with pregnancy, hypophysectomy, aneurysm, vascular malformation, thrombosis, infection, immunological disorder, and complication due to head trauma; a disorder associated with hypopituitarism including hypogonadism, Sheehan syndrome, diabetes insipidus, Kallman's disease, Hand-Schuller-Christian disease, Letterer-Siwe disease, sarcoidosis, empty sella syndrome, and dwarfism; a disorder associated with hyperpituitarism including acromegaly, giantism, and syndrome of inappropriate antidiuretic hormone (ADH) secretion (SIADH) often caused by benign adenoma; a disorder associated with hypothyroidism including goiter, myxedema, acute thyroiditis associated with bacterial infection, subacute thyroiditis associated with viral infection, autoimmune thyroiditis (Hashimoto's disease), and cretinism; a disorder associated with hyperthyroidism including thyrotoxicosis and its various forms, Grave's disease, pretibial myxedema, toxic multinodular goiter, thyroid carcinoma, and Plummer's disease; a disorder associated with hyperparathyroidism including Conn disease (chronic hypercalemia); a pancreatic disorder such as Type I or Type II diabetes mellitus and associated complications; a disorder associated with the adrenals such as hyperplasia, carcinoma, or adenoma of the adrenal cortex, hypertension associated with alacalosis, amyloidosis, hypokalemia, Cushing's disease, Liddle's syndrome, and Arnold-Healy-Gordon syndrome, pheochromocytoma tumors, and Addison's disease; a disorder associated with gonadal steroid hormones such as: in women, abnormal prolactin production, infertility, endometriosis, perturbation of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, a hypergonadal disorder associated with Leydig cell tumors, androgen resistance associated with absence of androgen receptors, syndrome of 5 a-reductase, and gynecomastia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a reproductive disorder, such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoirnmune disorders, an ectopic pregnancy, and teratogenesis; cancer of the breast, fibrocystic breast disease, and galactorrhea; a disruption of spermatogenesis, abnormal sperm physiology, benign prostatic hyperplasia, prostatitis, Peyronie's disease, and impotence; a cardiovascular disorder, such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation; and an infection such as that caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection such as that caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, and campylobacter, pseudomonas, vibrio, brucella, francisella, yersinia, bartonella, norcardium, actinomyces, mycobacterium, spirochaetale, rickettsia, chlamydia, or mycoplasma; an infection such as that caused by a fungal agent classified as aspergillus, blastomyces, dermatophytes, cryptococcus, coccidioides, malasezzia, histoplasma, or other fungal agents causing various mycoses; and an infection such as that caused by a parasite classified as plasmodium or malaria-causing, parasitic entamoeba, leishmania, trypanosoma, toxoplasma, pneumocystis carinii, intestinal protozoa such as giardia, trichomonas, tissue nematodes such as trichinella, intestinal nematodes such as ascaris, lymphatic filarial nematodes, trematodes such as schistosoma, or cestrodes such as tapeworm. Polynucleotides encoding EXMES may be used in Southern or northern analysis, dot blot, or other membrane-based technologies; in PCR technologies; in dipstick, pin, and multiformat ELISA-like assays; and in microarrays utilizing fluids or tissues from patients to detect altered EXMES expression. Such qualitative or quantitative methods are well known in the art.

In a particular aspect, polynucleotides encoding EXMES may be used in assays that detect the presence of associated disorders, particularly those mentioned above. Polynucleotides complementary to sequences encoding EXMES may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding EXMES in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of EXMES, a normal or standard profile for expression is established. This may be accomplished by combining body fluids or cell extracts taken from normal subjects, either animal or human, with a sequence, or a fragment thereof, encoding EXMES, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding EXMES may involve the use of PCR. These oligomers may be chemically synthesized, generated enzymatically, or produced in vitro. Oligomers will preferably contain a fragment of a polynucleotide encoding EXMES, or a fragment of a polynucleotide complementary to the polynucleotide encoding EXMES, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding EXMES may be used to detect single nucleotide polymorphisms (SNPs). SNPs are substitutions, insertions and deletions that are a frequent cause of inherited or acquired genetic disease in humans. Methods of SNP detection include, but are not limited to, single-stranded confornation polymorphism (SSCP) and fluorescent SSCP (fSSCP) methods. In SSCP, oligonucleotide primers derived from polynucleotides encoding EXMES are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isouiazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of EXMES include radiolabeling or biotinylating nucleotides, coamplification of a control nucleic acid, and interpolating results from standard curves. (See, e.g., Melby, P. C. et al. (1993) J. Imnunol. Methods 159:235-244; Duplaa, C. et al. (1993) Anal. Biochem 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, EXMES, fragments of EXMES, or antibodies specific for EXMES may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refmed when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for EXMES to quantify the levels of EXMES expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London.

In another embodiment of the invention, nucleic acid sequences encoding EXMES may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding EXMES on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, EXMES, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between EXMES and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with EXMES, or fragments thereof, and washed. Bound EXMES is then detected by methods well known in the art. Purified EXMES can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding EXMES specifically compete with a test compound for binding EXMES. In this manner, antibodies can be used to detect the presence of any peptide which shares one or more antigenic determinants with EXMES.

In additional embodiments, the nucleotide sequences which encode EXMES may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, including U.S. Ser. No. 60/301,789, U.S. Ser. No. 60/324,149, U.S. Ser. No. 60/327,713, U.S. Ser. No. 60/329,215, U.S. Ser. No. 60/340,218, U.S. Ser. No. 60/370,761, and U.S. Ser. No.60/373,824, mentioned above and below, are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chioroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genornics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programning, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicanis (Incyte Genomics, Palo Alto Calif.); hidden Markov model (HM)-based protein family databases such as PFAM, INCY, and TIGRFAM (Haft, D. H. et al. (2001) Nucleic Acids Res. 29:41-43); and H-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:23-44. Fragments from about 20 to about 4000 nucleotides which are useful in hybridization and amplification technologies are described in Table 4, column 2.

IV. Identification and Editing of Coding Sequences from Genomic DNA

Putative extracellular messengers were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set. to 30 kb. To determine which of these Genscan predicted cDNA sequences encode extracellular messengers, the encoded polypeptides were analyzed by querying against PFAM models for extracellular messengers. Potential extracellular messengers were also identified by homology to Incyte CDNA sequences that had been annotated as extracellular messengers. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example m. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example II were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of EXMES Encoding Polynucleotides

The sequences which were used to assemble SEQ ID NO:23-44 were compared with sequences from the Incyte LIFESEQ database and public domain databases using BLAST and other implementations of the Smith-Waterman algorithm. Sequences from these databases that matched SEQ ID NO:23-44 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LEFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignient. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding EXMES are analyzed with respect to the tissue sources from which they were derived. For example, some full length sequences are assembled, at least in part, with overlapping Incyte cDNA sequences (see Example 111). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding EXMES. cDNA sequences and cDNA library/tissue information are found in the LIFESEQ GOLD database (Incyte Genomics, Palo Alto Calif.).

VIII. Extension of EXMES Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C, 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 /μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C.in 384-well plates in LB/2x carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in EXMES Encoding Polynucleotides

Common DNA sequence variants known as single nucleotide polymorphisms (SNPs) were identified in SEQ ID NO:23-44 using the LIFESEQ database (Incyte Genomics). Sequences from the same gene were clustered together and assembled as described in Example II, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper timming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:23-44 are employed to screen cDNAs, genomic DNAs, or rnRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amershan Biosciences), and T4 polynucleotide kinase (DuPont NEN, Boston Mass.). The labeled oligonucleotides are substantially purified using a SEPHADEX G-25 superfme size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NBN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.) Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGIT kits (Incyte). Specific control poly(A)⁺ RNAs are synthesized by in vitro transcription from non-coding yeast genomic DNA. After incubation at 37° C. for 2 hr, each reaction sample (one with Cy3 and another with CyS labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then, deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas muitiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

Array elements that exhibited at least about a two-fold change in expression, a signal-to-background ratio of at least 2.5, and an element spot size of at least 40% were identified as differentially expressed using the GEMTOOLS program (Incyte Genomics).

Expression

For example, expression of SEQ ID NO:26 was downregulated in diseased tissue versus normal tissue as determined by microarray analysis. The gene expression profiles of normal brain tissue were compared to that of the amygdala, hippocampus, cerebellum, striatum, and cingulate of two patients with severe and one with mild Alzheimer's disease (AD). Expression of SEQ ID NO:26 was decreased in the amygdala of all three patients, in the hippocampus of one patient with severe AD and in that of the patient with mild AD, and in the cerebellum of the second patient with severe AD. Therefore, in various embodiments, SEQ ID NO:26 can be used for one or more of the following: i) monitoring treatment of Alzheimer's disease, ii) diagnostic assays for Alzheimer's disease, and iii) developing therapeutics and/or other treatments for Alzheimer's disease.

In a further example, expression of SEQ ID NO:29 and SEQ ID NO:32-34 were upregulated in treated versus untreated cells as determined by microarray analysis. In order to understand the molecular mechanisms underlying the phenotypic differences in epithelial cells grown in the presence or absence of serum, the gene expression profiles of MDA-mb-231 cells grown in the presence and absence of serum were compared. Expression of SEQ ID NO:29 and SEQ ID NO:32-34 was increased in the presence of serum. Therefore, in various embodiments, SEQ ID NO:29, encoding SEQ ID NO:7 and SEQ ID NO:32-34, encoding SEQ ID NO:10-12 respectively, can be used for one or more of the following: i) diagnostic assays to understand the molecular mechanisms underlying the phenotypic differences in epithelial cells grown in the presence and absence of serum.

For example, expression of SEQ ID NO:29 and SEQ ID NO:32-34 were downregulated in TNF-α treated cells versus untreated cells as determined by microarray analysis. HAECs were treated with TNF-α for 1, 2, 4, 6, 8, 10, 24, and 48 hours. These TNF-α treated cells were compared to untreated HAECs. Expression of SEQ ID NO:29 and SEQ ID NO:32-34 was decreased in TNPF-α treated cells after a minimum of 6 hours treatment and remained at that level up to 48 hours of treatment. Vascular tissue genes differentially expressed during treatment of HAECs with TNF-α may serve as markers of a wide range of both physiological and pathophysiological processes, such as vascular tone regulation, coagulation and thrombosis, atherosclerosis, inflammation, and some infectious diseases. Further, monitoring the endothelial cells' response to TNF-α at the level of the MnRNA expression can provide information necessary for better understanding of both TNF-signaling pathways and endothelial cell biology. Therefore, in various embodiments, SEQ ID NO:29, encoding SEQ ID NO:7 and SEQ ID NO:32-34, encoding SEQ ID NO:10-12 respectively, can be used for one or more of the following: i) monitoring treatment of vascular tone regulation, coagulation and thrombosis, atherosclerosis, inflammation, and some infectious diseases, ii) diagnostic assays for vascular tone regulation, coagulation and thrombosis, atherosclerosis, inflammation, and some infectious diseases, and iii) developing therapeutics and/or other treatments for vascular tone regulation, coagulation and thrombosis, atherosclerosis, inflammation, and some infectious diseases.

In an alternate example, expression of SEQ ID NO:29 and SEQ ID NO:32-34 were downregulated in TNF-α treated cells versus untreated cells as determined by microarray analysis. HUAECs were treated with TNF-α for 1, 2, 4, 8, and 24 hours. These TNF-α treated cells were compared to untreated HUAECs. Expression of SEQ ID NO:29 and SEQ ID NO:32-34 were downregulated in TNF-α treated cells after a minimum of 8 hours treatment and remained at that level up to 24 hours of treatment. Vascular tissue genes differentially expressed during treatment of HUAECs with TNF-α may serve as markers of a wide range of both physiological and pathophysiological processes, such as vascular tone regulation, coagulation and thrombosis, atherosclerosis, inflammation, and some infectious diseases. Further, monitoring the endothelial cells' response to TNF-α at the level of the mRNA expression can provide information necessary for better understanding of both TNF-α signaling pathways and endothelial cell biology. Therefore, in various embodiments, SEQ ID NO:29, encoding SEQ ID NO:7 and SEQ ID NO:32-34, encoding SEQ ID NO:10-12 respectively, can be used for one or more of the following: i) monitoring treatment of vascular tone regulation, coagulation and thrombosis, atherosclerosis, inflammation, and some infectious diseases, ii) diagnostic assays for vascular tone regulation, coagulation and thrombosis, atherosclerosis, inflammation, and some infectious diseases, and iii) developing therapeutics and/or other treatments for vascular tone regulation, coagulation and thrombosis, atherosclerosis, inflammation, and some infectious diseases.

In an alternate example, expression of SEQ ID NO:29, SEQ ID NO:32, and SEQ ID NO:34 was downregulated at least two fold in senescent cells as determined by microarray analysis. Therefore, in various embodiments, SEQ ID NO:29, encoding SEQ ID NO:7 and SEQ ID NO:32, encoding SEQ ID NO:10, and SEQ ID NO:34 encoding SEQ ID NO:12, can be used for one or more of the following: i) diagnostic assays for senescence, and ii) developing therapeutics and/or other treatments for senescence.

In an alternate example, expression of SEQ ID NO:29 and SEQ ID NO:32-34 were downregulated in tumorous lung tissue compared to that of normal lung tissue from matched donors as determined by microarray analysis. Expression of SEQ ID NO:29 and SEQ ID NO:32-34 was decreased in three out of eleven donors. Therefore, in various embodiments, SEQ ID NO:29 and SEQ ID NO:32-34 can be used for one or more of the following: i) monitoring treatment of lung cancer, ii) diagnostic assays for lung cancer, and iii), developing therapeutics and/or other treatments for lung cancer.

In a further example, expression of SEQ ID NO:35-37 was upregulated in tumorous lung tissue were compared to that of normal lung tissue from matched donors as determined by microarray analysis. SEQ ID NO:35-37 were found to be upregulated at least two fold in tumorous tissue from the same one out of eleven donors. Analysis of gene expression patterns associated with the development and progression of lung cancer can yield tremendous insight into the biology underlying this disease, and can lead to the development of improved diagnostics and therapeutics. Therefore, in various embodiments, SEQ ID NO:35-37, encoding SEQ ID NO:13-15 respectively, can be used for one or more of the following: i) monitoring treatment of lung cancer, ii) diagnostic assays for lung cancer, and iii) developing therapeutics and/or other treatments for lung cancer.

For example, expression of SEQ ID NO:41 was downregulated in cells treated with dexamethasone versus untreated cells as determined by microarray analysis. Early confluent C3A cells were treated with dexamethasone at 1, 10, and 100 μM for 1, 3, and 6 hours. The treated cells were compared to untreated early confluent C3A cells. Therefore, in various embodiments, SEQ ID NO:41 can be used for one or more of the following: i) monitoring treatment of asthma and other autoimmune/inflammation disorders, ii) diagnostic assays for asthma and other autoimmune/inflammation disorders, and iii) developing therapeutics and/or other treatments for asthma and other autoimmunefinflammation disorders.

As another example, expression of SEQ ID NO:41 was downregulated in ovarian tumor tissue versus normal ovarian tissue as determined by microarray analysis. A normal ovary from a 79 year-old female donor was compared to an ovarian tumor from the same donor (Huntsman Cancer Institute, Salt Lake City, Utah). Therefore, in various embodiments, SEQ ID NO:41 can be used for one or more of the following: i) monitoring treatment of ovarian cancer and other cell proliferative disorders, ii) diagnostic assays for ovarian cancer and other cell proliferative disorders, and iii) developing therapeutics and/or other treatments for ovarian cancer and other cell proliferative disorders.

XII. Complementary Polynucleotides

Sequences complementary to the EXMES-encoding sequences, or any parts thereof, are used to detect, decrease, or inhibit expression of naturally occurring EXMES. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of EXMES. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the EXMES-encoding transcript.

XIII. Expression of EXMES

Expression and purification of EXMES is achieved using bacterial or virus-based expression systems. For expression of EXMES in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express EXMES upon induction with isopropyl beta-D-thiogalactopyranoside (IPTG). Expression of EXMES in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding EXMES by either homologous recombination or bacterial-mediated transposition involving transfer plasrnid intermediates. Viral infectivity is maintained and the strong polyhedrin promoter drives high levels of cDNA transcription. Recombinant baculovirus is used to infect Spodontera frugiverda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, EXMBiS is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from EXMES at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified EXMES obtained by these methods can be used directly in the assays shown in Examples XVII, XVIII, XIX, and XX, where applicable.

XIV. Functional Assays

EXMES function is assessed by expressing the sequences encoding EXMES at physiologically elevated levels in mammalian cell culture systems. cDNA is subdloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 mg of recombinant vector are transiently transfected into a human cell line, for example, an endothelial or hematopoietic cell line, using either liposome formulations or electroporation. 1-2 mg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of EXMES on gene expression can be assessed using highly purified populations of cells transfected with sequences encoding EXMES and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y). mRNA can be purified from the cells using methods well known by those of skill in the art. Expression of mRNA encoding EXMES and other genes of interest can be analyzed by northern analysis or microarray techniques.

XV. Production of EXMES Specific Antibodies

EXMES substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the EXMES amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are imnmunized with the oligopeptide-KLH complex in complete Freund's adjuvant. Resulting antisera are tested for antipeptide and anti-EXMES activity by, for example, binding the peptide or EXMES to a substrate, blocking with 1% BSA, reacting with rabbit antisera, washing, and reacting with radio-iodinated goat anti-rabbit IgG.

XVI. Purification of Naturally Occurring EXMES Using Specific Antibodies

Naturally occurring or recombinant EXMES is substantially purified by immunoaffinity chromatography using antibodies specific for EXMES. An immunoaffinity column is constructed by covalently coupling anti-EXMES antibody to an activated chromatographic resin, such as CNBr-activated SEPHAROSE (Amersham Biosciences). After the coupling, the resin is blocked and washed according to the manufacturer's instructions.

Media containing EXMES are passed over the immunoaffinity column, and the column is washed under conditions that allow the preferential absorbance of EXMES (e.g., high ionic strength buffers in the presence of detergent). The column is eluted under conditions that disrupt antibody/EXMES binding (e.g., a buffer of pH 2 to pH 3, or a high concentration of a chaotrope, such as urea or thiocyanate ion), and EXMES is collected.

XVII. Identification of Molecules Which Interact with EXMES

EXMES, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled EXMES, washed, and any wells with labeled EXMES complex are assayed. Data obtained using different concentrations of EXMES are used to calculate values for the number, affinity, and association of EXMES with the candidate molecules.

Alternatively, molecules interacting with EXMES are analyzed using the yeast two-hybrid system as described in Fields, S. and O. Song (1989) Nature 340:245-246, or using commercially available kits based on the two-hybrid system, such as the MATCHMAKER system (Clontech).

EXMES may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of EXMES Activity

EXMES activity is measured by one of several methods. Growth factor activity is measured by the stimulation of DNA synthesis in Swiss mouse 3T3 cells. (McKay, I. and I. Leigh, eds. (1993) Growth Factors: A Practical Approach, Oxford University Press, New York, N.Y.) Initiation of DNA synthesis indicates the cells' entry into the mitotic cycle and their commitment to undergo later division. 3T3 cells are competent to respond to most growth factors, not only those that are mitogenic, but also those that are involved in embryonic induction. This competence is possible because the in vivo specificity demonstrated by some growth factors is not necessarily inherent but is determined by the responding tissue. In this assay, varying amounts of EXMES are added to quiescent 3T3 cultured cells in the presence of [³H]thymidine, a radioactive DNA precursor. EXMES for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold EXMES concentration range is indicative of growth factor activity. One unit of activity per milliliter is defined as the concentration of EXMES producing a 50% response level, where 100% represents maximal incorporation of [³H]thymidine into acid-precipitable DNA.

Alternatively, an assay for cytokine activity measures the proliferation of leukocytes. In this assay, the amount of tritiated thymidine incorporated into newly synthesized DNA is used to estimate proliferative activity. Varying amounts of EXMES are added to cultured leukocytes, such as granulocytes, monocytes, or lymphocytes, in the presence of [³H]thymidine, a radioactive DNA precursor. EXMES for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold EXMES concentration range is indicative of EXMES activity. One unit of activity per milliliter is conventionally defined as the concentration of EXMES producing a 50% response level, where 100% represents maximal incorporation of [³H]thymidine into acid-precipitable DNA.

An alternative assay for EXMES cytokine activity utilizes a Boyden micro chamber (Neuroprobe, Cabin John MD) to measure leukocyte chemotaxis (Vicari, A. P. et al. (1997) Immunity 7:291-301). In this assay, about 10⁵ migratory cells such as macrophages or monocytes are placed in cell culture media in the upper compartment of the chamber. Varying dilutions of EXMES are placed in the lower compartment. The two compartments are separated by a 5 or 8 micron pore polycarbonate filter (Nucleopore, Pleasanton Calif.). After incubation at 37° C. for 80 to 120 minutes, the filters are fixed in methanol and stained with appropriate labeling agents. Cells which migrate to the other side of the filter are counted using standard microscopy. The chemotactic index is calculated by dividing the number of migratory cells counted when EXMES is present in the lower compartment by the number of migratory cells counted when only media is present in the lower compartment. The chemotactic index is proportional to the activity of EXMES.

Alternatively, cell lines or tissues transformed with a vector encoding EXMES can be assayed for EXMES activity by immunoblotting. Cells are denatured in SDS in the presence of ,β-mercaptoethanol, nucleic acids removed by ethanol precipitation, and proteins purified by acetone precipitation. Pellets are resuspended in 20 mM tris buffer at pH 7.5 and incubated with Protein G-Sepharose pre-coated with an antibody specific for EXMES. After washing, the Sepharose beads are boiled in electrophoresis sample buffer, and the eluted proteins subjected to SDS-PAGE. The SDS-PAGE is transferred to a nitrocellulose membrane for immunoblotting, and the EXMES activity is assessed by visualizing and quantifying bands on the blot using the antibody specific for EXMEES as the primary antibody and ¹²⁵I-labeled IgG specific for the primary antibody as the secondary antibody.

Alternatively, an assay for EXMES activity measures the amount of EXMES in secretory, membrane-bound organelles. Transfected cells as described above are harvested and lysed. The lysate is fractionated using methods known to those of skill in the art, for example, sucrose gradient ultracentrifugation. Such methods allow the isolation of subcellular components such as the Golgi apparatus, ER, small membrane-bound vesicles, and other secretory organelles. lmmunoprecipitations from fractionated and total cell lysates are performed using EXMES-specific antibodies, and imrnunoprecipitated samples are analyzed using SDS-PAGE and immunoblotting techniques. The concentration of EXMES in secretory organelles relative to EXMES in total cell lysate is proportional to the amount of EXMES in transit through the secretory pathway.

Alternately, an assay for BXMES activity measures its inhibitory activity on Hepatocyte Growth Factor (HGF) activator. In this assay, HGF activator (450 ng/ml) is mixed with various concentrations of purified EXMES in PBS containing 0.05% CHAPS and incubated at 37 degrees C. for 30 minutes to form an enzyme-inhibitor complex. The remaining HGP-converting activity in the mixture is measured by the addition of equal amounts of single chain HGF (sc-HGO) (1.5 μg/ml in PBS containing 0.05% CHAPS) and dextran sulfate (100 mg/ml, MWCO=500,000, Sigma) followed by further incubation for 2 hours, and subsequent, analysis by SDS-PAGE under reducing gel conditions. The gel is stained with coomassie blue and the amounts of sc-HGF and the heterodimeric form are measured by scanning the stained bands. The inhibitory activity of EXMES against HGF activator is estimated by calculating the ratio of the remaining single chain form to total HGF (Shimomura, T. et al. (1997) J. Biol. Chem. 272:6370-6376).

Alternatively, an assay for EXMES activity measures the stimulation or inhibition of neurotransmission in cultured cells. Cultured CHO fibroblasts are exposed to ENS. Following endocytic uptake of EXMES, the cells are washed with fresh culture medium, and a whole cell voltage-clamped Xenopus myocyte is manipulated into contact with one of the fibroblasts in EXMES-free medium. Membrane currents are recorded from the myocyte. Increased or decreased current relative to control values are indicative of neuromodulatory effects of EXMES (Morimoto, T. et al. (1995) Neuron 15:689-696).

Alternatively, AMP binding activity is measured by combining EXMES with ³²P-labeled AMP. The reaction is incubated at 37° C. and terminated by addition of trichloroacetic acid. The acid extract is neutralized and subjected to gel electrophoresis to remove unbound label. The radioactivity retained in the gel is proportional to EXMES activity.

XIX. EXMES Secretion Assay

A high throughput assay may be used to identify polypeptides that are secreted in eukaryotic cells. In an example of such an assay, polypeptide expression libraries are constructed by fusing 5′-biased cDNAs to the 5′-end of a leaderless β-lactamase gene. β-lactamase is a convenient genetic reporter as it provides a high signal-to-noise ratio against low endogenous background activity and retains activity upon fusion to other proteins. A dual promoter system allows the expression of β-lactamase fusion polypeptides in bacteria or eukaryotic cells, using the lac or CMV promoter, respectively.

Libraries are first transformed into bacteria, e.g., E. coli, to identify library members that encode fusion polypeptides capable of being secreted in a prokaryotic system. Mammalian signal sequences direct the translocation of β-lactamase fusion polypeptides into the periplasm of bacteria where it confers antibiotic resistance to carbenicillin. Carbenicillin-selected bacteria are isolated on solid media, individual clones are grown in liquid media, and the resulting cultures are used to isolate library member plasmid DNA.

Mammalian cells, e.g., 293 cells, are seeded into 96-well tissue culture plates at a density of about 40,000 cells/well in 100 μl phenol red-free DME supplemented with 10% fetal bovine serum (FBS) (Life Technologies, Rockville, Md.). The following day, purified plasmid DNAs isolated from carbenicillin-resistant bacteria are diluted with 15 μl OPTI-MEM I medium (Life Technologies) to a volume of 25 μl for each well of cells to be transfected. In separate plates, 1 lt LF2000 Reagent (Life Technologies) is diluted into 25 μl/well OPTI-MEM I. The 25 μl diluted LF2000 Reagent is then combined with the 25 μl diluted DNA, mixed briefly, and incubated for 20 minutes at room temperature. The resulting DNA-LF2000 reagent complexes are then added directly to each well of 293 cells. Cells are also transfected with appropriate control plasmids expressing either wild-type β-lactamase, leaderless β-lactamase, or, for example, CD4-fused leaderless β-lactamase. 24 hrs following transfection, about 90 μl of cell culture media are assayed at 37° C. with 100 μM Nitrocefin (Calbiochem, San Diego, Calif.) and 0.5 mM oleic acid (Sigma Corp. St. Louis, Mo.) in 10 mM phosphate buffer (pH 7.0). Nitrocefin is a substrate for β-lactamase that undergoes a noticeable color change from yellow to red upon hydrolysis. β-lactamase activity is monitored over 20 min in a microtiter plate reader at 486 mm. Increased color absorption at 486 nm corresponds to secretion of a β-lactamase fusion polypeptide in the transfected cell media, resulting from the presence of a eukaryotic signal sequence in the fusion polypeptide. Polynucleotide sequence analysis of the corresponding library member plasmid DNA is then used to identify the signal sequence-encoding cDNA. (Described in U.S. patent application Ser. No. 09/803,317, filed Mar. 9, 2001.)

For example, SEQ ID NO:4 was shown to be a secreted protein using this assay.

XX. Demonstration of Immunoglobulin Activity

An assay for EXMES activity measures the ability of EXMES to recognize and precipitate antigens from serum This activity can be measured by the quantitative precipitin reaction. (Golub, E. S. et al. (1987) Immunology: A Synthesis, Sinauer Associates, Sunderland, Mass., pages 113-115.) EXMES is isotopically labeled using methods known in the art. Various serum concentrations are added to constant amounts of labeled EXMES. EXMES-antigen complexes precipitate out of solution and are collected by centrifugation. The amount of precipitable EXMES-antigen complex is proportional to the amount of radioisotope detected in the precipitate. The amount of precipitable EXMES-antigen complex is plotted against the serum concentration. For various serum concentrations, a characteristic precipitin curve is obtained, in which the amount of precipitable EXMES-antigen complex initially increases proportionately with increasing serum concentration, peaks at the equivalence point, and then decreases proportionately with further increases in serum concentration. Thus, the amount of precipitable EXMES-antigen complex is a measure of EXMES activity which is characterized by sensitivity to both limiting and excess quantities of antigen.

Alternatively, an assay for EXMES activity measures the expression of EXMES on the cell surface. cDNA encoding EXMES is transfected into a non-leukocytic cell line. Cell surface proteins are labeled with biotin (de la Fuente, M. A. et al. (1997) Blood 90:2398-2405). Immunoprecipitations are performed using EXMES-specific antibodies, and immunoprecipitated samples are analyzed using SDS-PAGE and immunoblotting techniques. The ratio of labeled immunoprecipitant to unlabeled imnmunoprecipitant is proportional to the amount of EXMES expressed on the cell surface.

Alternatively, an assay for EXMES activity measures the amount of cell aggregation induced by overexpression of EXMES. In this assay, cultured cells such as NIH3T3 are transfected with cDNA encoding EXMES contained within a suitable marmnalian expression vector under control of a strong promoter. Cotransfection with cDNA encoding a fluorescent marker protein, such as Green Fluorescent Protein (CLONTECH), is useful for identifying stable transfectants. The amount of cell agglutination, or clumping, associated with transfected cells is compared with that associated with untransfected cells. The amount of cell agglutination is a direct measure of EXMES activity.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | |
| Incyte Project ID | SEQ ID NO: | Polypeptide ID | SEQ ID NO: | ID | Incyte Full Length Clones |
| 7497502 | 1 | 7497502CD1 | 23 | 7497502CB1 | |
| 7103532 | 2 | 7103532CD1 | 24 | 7103532CB1 | |
| 7500108 | 3 | 7500108CD1 | 25 | 7500108CB1 | 90051308CA2, 90051348CA2 |
| 7500665 | 4 | 7500665CD1 | 26 | 7500665CB1 | 90125051CA2, 90125067CA2, |
| | | | | | 90125083CA2 |
| 3569792 | 5 | 3569792CD1 | 27 | 3569792CB1 | |
| 7500100 | 6 | 7500100CD1 | 28 | 7500100CB1 | 90028512CA2, 90028520CA2 |
| 5201851 | 7 | 5201851CD1 | 29 | 5201851CB1 | |
| 7500667 | 8 | 7500667CD1 | 30 | 7500667CB1 | |
| 7744055 | 9 | 7744055CD1 | 31 | 7744055CB1 | |
| 7502082 | 10 | 7502082CD1 | 32 | 7502082CB1 | |
| 7502084 | 11 | 7502084CD1 | 33 | 7502084CB1 | |
| 7502085 | 12 | 7502085CD1 | 34 | 7502085CB1 | |
| 7502093 | 13 | 7502093CD1 | 35 | 7502093CB1 | |
| 7502097 | 14 | 7502097CD1 | 36 | 7502097CB1 | |
| 7502108 | 15 | 7502108CD1 | 37 | 7502108CB1 | |
| 7500668 | 16 | 7500668CD1 | 38 | 7500668CB1 | |
| 7505114 | 17 | 7505114CD1 | 39 | 7505114CB1 | 5523059CA2, 90017347CA2, |
| | | | | | 90118925CA2, 90119009CA2, |
| | | | | | 90119025CA2, 90130340CA2, |
| | | | | | 90130456CA2, 90130480CA2 |
| 7506452 | 18 | 7506452CD1 | 40 | 7506452CB1 | 90117542CA2 |
| 7506730 | 19 | 7506730CD1 | 41 | 7506730CB1 | 90111904CA2 |
| 7505046 | 20 | 7505046CD1 | 42 | 7505046CB1 | |
| 7506453 | 21 | 7506453CD1 | 43 | 7506453CB1 | |
| 7509967 | 22 | 7509967CD1 | 44 | 7509967CB1 | |

TABLE 2

| | | GenBank ID NO, | | |
|---|---|---|---|---|
| Polypeptide | Incyte | or PROTEOME | Probability | |
| SEQ ID NO, | Polypeptide ID | ID NO, | Score | Annotation |
| 1 | 7497502CD1 | g1311661 | 0.0 | [Homo sapiens] hepatocyte growth factor-like protein |
| | | | | Waltz, S. E. et al. Hepatocyte nuclear factor-4 is responsible for the liver-specific |
| | | | | expression of the gene coding for hepatocyte growth factor-like protein. J. Biol. |
| | | | | Chem. 271, 9024-9032 (1996) |
| 2 | 7103532CD1 | g10998440 | 7.5E−183 | [Mus musculus] EGF-related protein SCUBE1 |
| | | | | Grimmond, S. et al. Cloning, Mapping, and Expression Analysis of a Gene |
| | | | | Encoding a Novel Mammalian EGF-Related Protein (SCUBE1). Genomics 70 (1), |
| | | | | 74-81 (2000) |
| 3 | 7500108CD1 | g339548 | 3.8E−178 | [Homo sapiens] transforming growth factor-beta 1 binding protein precursor |
| | | | | Kanzaki, T. et al. (1990) Cell 61 (6), 1051-1061 |
| 4 | 7500665CD1 | g338051 | 5.3E−205 | [Homo sapiens] secretogranin II |
| | | | | Gerdes, H.-H. et al. (1989) J. Biol. Chem. 264, 12009-12015 |
| 5 | 3569792CD1 | g10998440 | 0.0 | [Mus musculus] EGF-related protein SCUBE1 |
| | | | | Grimmond, S. et al. (2000) Genomics 70 (1), 74-81 |
| 6 | 7500100CD1 | g12654463 | 4.0E−96 | [Homo sapiens] (BC001059) chromogranin A (parathyroid secretory protein 1) |
| 7 | 5201851CD1 | g19909128 | 0.0 | [Homo sapiens] transforming growth factor-beta binding protein-1S |
| 8 | 7500667CD1 | g338051 | 6.2E−268 | [Homo sapiens] secretogranin II |
| | | | | Gerdes, H.-H. et al. supra |
| 9 | 7744055CD1 | g7362977 | 2.2E−129 | [Homo sapiens] neuroendocrine secretory protein 55 |
| | | | | Hayward, B. E. et al. (2000) Hum. Mol. Genet. 9 (5), 835-841 |
| 10 | 7502082CD1 | g19909128 | 0.0 | [Homo sapiens] transforming growth factor-beta binding protein-1S |
| 11 | 7502084CD1 | p19909128 | 0.0 | [Homo sapiens] transforming growth factor-beta binding protein-1S |
| 12 | 7502085CD1 | g19909128 | 0.0 | [Homo sapiens] transforming growth factor-beta binding protein-1S |
| 13 | 7502093CD1 | g19909128 | 0.0 | [Homo sapiens] transforming growth factor-beta binding protein-1S |
| | | 339486\|LTBP1 | 0.0 | [Homo sapiens][Small molecule-binding protein] Latent transforming growth |
| | | | | factor beta binding protein, contains cysteine rich and EGF-like repeats, involved |
| | | | | in assembly and secretion of latent TGF-beta |
| | | 619058\|Ltbp1 | 0.0 | [Rattus norvegicus][Inhibitor or repressor] Protein with EGF-like and cysteine |
| | | | | rich repeats that is a component of masking protein, which inhibits TGF-beta 1 |
| | | | | and is expressed in tissues which express TGF-beta-1 |
| | | 609294\|Ltbp1 | 0.0 | [Mus musculus][Small molecule-binding protein] Protein with strong similarity to |
| | | | | human LTBP1, which is involved in assembly and secretion of TGF-beta, has very |
| | | | | strong similarity to rat Rn.11340, which is expressed in tissues which express TGF |
| | | | | beta 1 |
| | | 617838\|LTBP3 | 2.6E−225 | [Homo sapiens] Latent transforming growth factor-beta-binding protein-3, part of |
| | | | | the latent TGF-beta complexin platelets |
| | | 624508\|Ltbp2 | 5.9E−224 | [Rattus norvegicus] Protein with strong similarity to latent transforming growth |
| | | | | factor beta binding proteins, which target latent TGF-beta to the extracellular |
| | | | | matrix, contains a TB (8 cysteine) domain, contains EGF-like domains |
| 14 | 7502097CD1 | g339548 | 0.0 | [Homo sapiens] transforming growth factor-beta 1 binding protein precursor |
| | | | | (Kanzaki, T. et al (1990) Cell 61 (6), 1051-1061) |
| | | 339486\|LTBP1 | 0.0 | [Homo sapiens][Small molecule-binding protein] Latent transforming growth |
| | | | | factor beta binding protein, contains cysteine rich and EGF-like repeats, involved |
| | | | | in assembly and secretion of latent TGF-beta |
| | | 619058\|Ltbp1 | 0.0 | [Rattus norvegicus][Inhibitor or repressor] Protein with EGF-like and cysteine |
| | | | | rich repeats that is a component of masking protein, which inhibits TGF-beta 1 |
| | | | | and is expressed in tissues which express TGF-beta-1 |
| | | 609294\|Ltbp1 | 0.0 | [Mus musculus][Small molecule-binding protein] Protein with strong similarity to |
| | | | | human LTBP1, which is involved in assembly and secretion of TGF-beta, has very |
| | | | | strong similarity to rat Rn.11340, which is expressed in tissues which express TGF |
| | | | | beta 1 |
| | | 624508\|Ltbp2 | 5.3E−244 | [Rattus norvegicus] Protein with strong similarity to latent transforming growth |
| | | | | factor beta binding proteins, which target latent TGF-beta to the extracellular |
| | | | | matrix, contains a TB (8 cysteine) domain, contains EGF-like domains |
| | | 418532\|Ltbp2 | 1.6E−242 | [Mus musculus][Structural protein] Latent TGF-beta binding protein, may |
| | | | | assemble latent TGF-beta complexes in developing elastic tissues, contains |
| | | | | proline/glycine-rich sequences alternating with cysteine-rich clusters, expressed in |
| | | | | embryonic cartilage perichondrium and blood vessel |
| 15 | 7502108CD1 | g19909128 | 0.0 | [Homo sapiens] transforming growth factor-beta binding protein-1S |
| | 7502108CD1 | 339486\|LTBP1 | 0.0 | [Homo sapiens][Small molecule-binding protein] Latent transforming growth |
| | | | | factor beta binding protein, contains cysteine rich and EGF-like repeats, involved |
| | | | | in assembly and secretion of latent TGF-beta |
| | 7502108CD1 | 619058\|Ltbp1 | 0.0 | [Rattus norvegicus][Inhibitor or repressor] Protein with EGF-like and cysteine |
| | | | | rich repeats that is a component of masking protein, which inhibits TGF-beta 1 |
| | | | | and is expressed in tissues which express TGF-beta-1 |
| | 7502108CD1 | 609294\|Ltbp1 | 0.0 | [Mus musculus][Small molecule-binding protein] Protein with strong similarity to |
| | | | | human LTBP1, which is involved in assembly and secretion of TGF-beta, has very |
| | | | | strong similarity to rat Rn.11340, which is expressed in tissues which express TGF |
| | | | | beta 1 |
| | 7502108CD1 | 624508\|Ltbp2 | 1.3e−240 | [Rattus norvegicus] Protein with strong similarity to latent transforming growth |
| | | | | factor beta binding proteins, which target latent TGF-beta to the extracellular |
| | | | | matrix, contains a TB (8 cysteine) domain, contains EGF-like domains |
| | 7502108CD1 | 339488\|LTBP2 | 9.3e−236 | [Homo sapiens][Regulatory subunit; Anchor Protein; Inhibitor orrepressor; Small |
| | | | | molecule-binding protein][Extracellular matrix(cuticle and basement |
| | | | | membrane); Extracellular (excluding cellwall)] Latent transforming growth factor |
| | | | | (TGF)-beta binding protein, required for secretion and processing of latent TGF- |
| | | | | beta, targets latent TGF-beta to the extracellular matrix |
| 16 | 7500668CD1 | g338051 | 1.0E−32 | [Homo sapiens] secretogranin II |
| | | | | Gerdes, H.-H.et al. (1989) J. Biol. Chem. 264, 12009-12015 |
| | | | | The primary structure of human secretogranin II, a widespread tyrosine-sulfated |
| | | | | secretory granule protein that exhibits low ph- and calcium-induced aggregation. |
| | — | 337880\|SCG2 | 9.3E−34 | [Homo sapiens] [Secretory vesicles; Cytoplasmic] Secretogranin II (chromogranin |
| | | | | C), precursor of the neuropeptide secretoneurin, localized within secretory |
| | | | | granules of endocrine cells and neurons; acts as a chemoattract influencing |
| | | | | eosinophil migration; downregulated in the rheumatoid joint |
| | | | | Eder, U. et al. (1997) Neurosci. Lett. 224, 139-141 |
| | | | | The presence of secretoneurin in human synovium and synovial fluid. |
| | | 581273\|Scg2 | 3.6E−27 | [Mus musculus] [Secretory vesicles; Cytoplasmic] Secretogranin II, member of |
| | | | | the granin (chromogranin/secretogranin) protein family, a tyrosine-sulfated |
| | | | | secretory protein located in endocrine and neuron secretory granules; expression |
| | | | | is downregulated by cocaine. |
| 17 | 7505114CD1 | g307064 | 1.6E−66 | [Homo sapiens] interleukin 7 precursor |
| | | | | Goodwin, R. G. et al. (1989) Human interleukin 7, molecular cloning and growth |
| | | | | factor activity on human and murine B-lineage cells. Proc. Natl. Acad. Sci. U.S.A. |
| | | | | 86, 302-306 |
| | | 336016\|IL7 | 1.4E−67 | [Homo sapiens] [Ligand] Interleukin 7, a hematopoietic growth factor required for |
| | | | | nomral growth and development of B cells and T cells |
| | | | | Chou, Y. K. (1999) IL-7 enhances Ag-specific human T cell response by |
| | | | | increasing expression of IL-2R alpha and gamma chains. J. Neuroimmunol. 96, |
| | | | | 101-111 |
| | | 583379\|I17 | 1.6E−25 | [Mus musculus] [Ligand] Interleukin 7, a hematopoietic growth factor required for |
| | | | | normal growth and development of B cells and T cells, induces T cell-mediated |
| | | | | anti-tumor response |
| | | 331142\|I17 | 1.4E−24 | [Rattus norvegicus] [Ligand] Interleukin 7, a hematopoietic growth factor that is |
| | | | | involved in the growth and development of B cells |
| 18 | 7506452CD1 | g531103 | 6.6E−82 | [Homo sapiens] prolactin |
| | | | | Hiraoka, Y. et al. (1991) Mol. Cell. Endocrinol. 75, 71-80 |
| | | | | A placenta-specific 5′ non-coding exon of human prolactin. |
| | | 337222\|PRL | 5.0E−88 | [Homo sapiens] [Ligand] [Extracellular (excluding cell wall)] Prolactin, a growth |
| | | | | hormone that stimulates lactation, has roles in angiogenesis inhibition and control |
| | | | | of cell proliferation, may function as an immunoregulator |
| | | | | Melck, D. et al. (2000) Endocrinology 141, 118-126 |
| | | | | Suppression of nerve growth factor Trk receptors and prolactin receptors by |
| | | | | endocannabinoids leads to inhibition of human breast and prostate cancer cell |
| | | | | proliferation. |
| | | 430628\|Prl | 9.9E−53 | [Rattus norvegicus] [Ligand] [Extracellular (excluding cell wall)] Prolactin, a |
| | | | | growth hormone-related protein, stimulates lactation, may mediate expression of |
| | | | | maternal behavior, may function as an immunoregulator with roles in control of |
| | | | | cell proliferation, involved induction of apoptosis and inhibition of angiongenesis |
| | | 582503\|Pl2 | 1.4E−28 | [Mus musculus] [Extracellular (excluding cell wall)] Placental lactogen II, a |
| | | | | member of the prolactin gene family, a secreted hormone that stimulates insulin |
| | | | | secretion from neonatal islet cells |
| 19 | 7506730CD1 | g13938105 | 1.4E−58 | [Mus musculus] Similar to neurexophilin 3 |
| | | 624404\|Nph3 | 2.5E−59 | [Rattus norvegicus] [Ligand] Protein with very strong similarity to human |
| | | | | NXPH3, which is a member of a family of secreted neuronal glycoproteins that |
| | | | | may function as ligands for alpha-neurexins |
| | | | | Missler, M. J et al. (1998) J. Biol. Chem. 273, 34716-34723 |
| | | | | Neurexophilin binding to alpha-neurexins. A single LNS domain functions as an |
| | | | | independently folding ligand-binding unit. |
| | | 735201\|NXPH3 | 7.1E−46 | [Homo sapiens] [Ligand] Neurexophilin, a member of a family of neuronal |
| | | | | glycoproteins that may function as ligands for alpha-neurexins |
| | | | | Missler, M., and Sudhof, T. C. (1998) J. Neurosci. 18, 3630-3638 |
| | | | | Neurexophilins form a conserved family of neuropeptide-like glycoproteins. |
| 20 | 7505046CD1 | g339552 | 7.4E−51 | [Homo sapiens] transforming growth factor-beta3 |
| | | | | ten Dijke, P. et al. (1988) Identification of another member of the transforming |
| | | | | growth factor type beta gene family. Proc. Natl. Acad. Sci. U.S.A. 85, 4715-4719 |
| | | 338482\|TGFB3 | 6.5E−52 | [Homo sapiens] [Ligand] Transforming growth factor-beta 3, member of a family |
| | | | | of cytokines that transmit their signals through transmembrane serine-threonine |
| | | | | kinases, involved in histogenesis and organogenesis; implicated in cleft lip, |
| | | | | tumorogenesis and preeclamptic pregnancy |
| | | | | Kaartinen, V. et al. (1995) Abnormal lung development and cleft palate in mice |
| | | | | lacking TGF-beta 3 indicates defects of epithelial-mesenchymal interaction. Nat. |
| | | | | Genet. 11, 415-421 |
| | | 329012\|Tgfb3 | 8.5E−50 | [Rattus norvegicus] [Ligand] Transforming growth factor-beta 3, member of a |
| | | | | family of cytokines, that transmit their signals through serine-threonine kinases, |
| | | | | involved in histogenesis, organogenesis, development and may play a role in |
| | | | | neuronal survival |
| 21 | 7506453CD1 | g34211 | 5.3E−17 | [Homo sapiens] reading frame prolactin |
| | | | | Cooke, N. E. et al. (1981) Human prolactin. cDNA structural analysis and |
| | | | | evolutionary comparisons. J. Biol. Chem. 256, 4007-4016 |
| | | 337222\|PRL | 4.2E−18 | [Homo sapiens][Ligand][Extracellular (excluding cell wall)] Prolactin, a growth |
| | | | | hormone that stimulates lactation, has roles in angiogenesis inhibition and control |
| | | | | of cell proliferation, may function as an immunoregulator |
| | | | | Burks, D. J. et al. (2000) IRS-2 pathways integrate female reproduction and |
| | | | | energy homeostasis. Nature 407, 377-82 |
| | | 430628\|Prl | 4.7E−07 | [Rattus norvegicus][Ligand][Extracellular (excluding cell wall)] Prolactin, a |
| | | | | growth hormone-related protein, stimulates lactation, may mediate expression of |
| | | | | maternal behavior, may function as an immunoregulator with roles in control of |
| | | | | cell proliferation, involved induction of apoptosis and inhibition of angiongenesis |
| | | | | Wilson, D. M. 3d et al. (1992) Prolactin message in brain and pituitary of adult |
| | | | | male rats is identical, PCR cloning and sequencing of hypothalamic prolactin |
| | | | | cDNA from intact and hypophysectomized adult male rats. Endocrinology 131, |
| | | | | 2488-90 |
| 22 | 7509967CD1 | g34211 | 2.3E−83 | [Homo sapiens] reading frame prolactin |
| | | 337222\|PRL | 2.0E−84 | [Homo sapiens][Ligand][Extracellular (excluding cell wall)] Prolactin, a growth |
| | | | | hormone that stimulates lactation, has roles in angiogenesis inhibition and control |
| | | | | of cell proliferation, may function as an immunoregulator. |
| | | | | Llovera, M. et al. (2000) Human prolactin (hPRL) antagonists inhibit hPRL- |
| | | | | activated signaling pathways involved in breast cancer cell proliferation. |
| | | | | Oncogene 19, 4695-705 |
| | | 430628\|Prl | 9.3E−48 | [Rattus norvegicus][Ligand][Extracellular (excluding cell wall)] Prolactin, a |
| | | | | growth hormone-related protein, stimulates lactation, may mediate expression of |
| | | | | maternal behavior, may function as an immunoregulator with roles in control of |
| | | | | cell proliferation, involved induction of apoptosis and inhibition of angiongenesis. |
| | | | | Piroli, G. G. et al. (2001) Progestin Regulation of Galanin and Prolactin Gene |
| | | | | Expression in Oestrogen-Induced Pituitary Tumours. J. Neuroendocrinol. 13, 302-309 |

TABLE 3

| | | Amino | | | | |
|---|---|---|---|---|---|---|
| SEQ | Incyte | Acid | Potential | Potential | | |
| ID | Polypeptide | Res- | Phosphorylation | Glycosylation | | Analytical Methods |
| NO: | ID | idues | Sites | Sites | Signature Sequences, Domains and Motifs | and Databases |
| 1 | | | | | PROTEIN GROWTH HEPATOCYTE FACTOR | BLAST_PRODOM |
| | | | | | LIKE PRECURSOR SIGNAL MACROPHAGE | |
| | | | | | STIMULATORY MSP HOMOLOG PD007364: H50-T123 | |
| | | | | | PRECURSOR SIGNAL SERINE GLYCOPROTEIN | BLAST_PRODOM |
| | | | | | PROTEASE KRINGLE HYDROLASE PLASMA | |
| | | | | | GROWTH PLASIENOGEN PD000395: S296-C375, | |
| | | | | | D383-C462, C200-C282, C124-C200 | |
| | | | | | PROTEASE SERINE PRECURSOR SIGNAL | BLAST_PRODOM |
| | | | | | HYDROLASE ZYMOGEN GLYCOPROTEIN | |
| | | | | | FAMILY MULTIGENE FACTOR PD000046: Q557-I718 | |
| | | | | | PROTEIN HEPATOCYTE GROWTH FACTOR | BLAST_PRODOM |
| | | | | | LIKE PRECURSOR SIGNAL MACROPHAGE | |
| | | | | | STIMEULATORY MSP KRINGLE PD012913: M15-Q49 | |
| | | | | | TRYPSIN DM00018\|P26927\|481-707: K495-M722 | BLAST_DOMO |
| | | | | | KRINGLE DM00069 | BLAST_DOMO |
| | | | | | \|P26927\|360-450: R374-D465, R281-D378, C124-E202, | |
| | | | | | R201-E285 | |
| | | | | | \|P26927\|96-186: G110-R201, C384-C462, S296-C375. | |
| | | | | | C205-C282 | |
| | | | | | \|P26927\|270-358: S284-R373, R201-C277, C384-Y456, | |
| | | | | | T123-C195 | |
| | | | | | Kringle domain signature F170-D175 Y253-D258 | MOTIFS |
| | | | | | F345-D350 F432-D437 | |
| 2 | 7103532CD1 | 919 | S68 S72 S227 S251 | N266 N451 | signal_cleavage: M1-G37 | SPSCAN |
| | | | S269 S361 S421 | N579 N610 | | |
| | | | S442 S446 S456 | N681 N710 | | |
| | | | S540 S560 S664 | N720 | | |
| | | | S711 S792 S816 | | | |
| | | | S830 T112 T258 | | | |
| | | | T296 T320 T406 | | | |
| | | | T412 T469 T501 | | | |
| | | | T511 T565 T684 | | | |
| | | | T713 T798 T813 | | | |
| | | | T840 T891 | | | |
| | | | | | Signal Peptide: M1-L28, M1-A31 | HMMER |
| | | | | | EGF-like domain: C90-C126, C368-C401, C327-C362, | HMMER_PFAM |
| | | | | | C217-C252, C49-C84, C132-C167, C286-C321, | |
| | | | | | C177-C213 | |
| | | | | | CUB domain: C729-Y838 | HMMER_PFAM |
| | | | | | Transmembrane domain: R8-R36 | TMAP |
| | | | | | N-terminus is non-cytosolic | |
| | | | | | Anaphylatoxin domain proteins BL01177: S238-L253, | BLIMPS_BLOCKS |
| | | | | | G96-F114, L316-G333, H336-C362 | |
| | | | | | GLYCOPROTEIN THYROGLOBULIN | BLAST_PRODOM |
| | | | | | PRECURSOR REPEAT THYROID HORMONE | |
| | | | | | IODINATION SIGNAL EGF-LIKE PROTEIN | |
| | | | | | PD009765: C574-G730, C558-C724 | |
| | | | | | GLYCOPROTEIN DOMAIN EGF-LIKE PROTEIN | BLAST_PRODOM |
| | | | | | PRECURSOR SIGNAL RECEPTOR INTRINSIC | |
| | | | | | FACTOR B12 REPEAT PD000165: C729-Y841 | |
| | | | | | EGF-LIKE DOMAIN DM00864\|I55476\|159-241: | BLAST_DOMO |
| | | | | | N290-D371, R330-V404, N95-C167, L61-N135 | |
| | | | | | EGF DM00003 | BLAST_DOMO |
| | | | | | \|P98163\|1373-1460: C98-C167, G293-V365 | |
| | | | | | \|JC4180\|148-206: G318-L370 | |
| | | | | | \|P53813\|148-206: G318-L370 | |
| | | | | | Aspartic acid and asparagine hydroxylation site: C62-C73 | MOTIFS |
| | | | | | C102-C113 C143-C154 C338-C349 C378-C389 | |
| | | | | | EGF-like domain signature 2: C71-C84 C111-C126 | MOTIFS |
| | | | | | C152-C167 C198-C213 C306-C321 C347-C362 | |
| | | | | | C387-C401 | |
| | | | | | Calcium-binding EGF-like domain pattern signature: | MOTIFS |
| | | | | | D45-C71 D86-C111 D128-C152 D323-C347 D364-C387 | |
| 3 | 7500108CD1 | 350 | S105 S131 S245 | N21 | signal_cleavage: M1-S20 | SPSCAN |
| | | | S316 S328 S347 | | | |
| | | | T23 T85 T175 | | | |
| | | | T211 T284 T288 | | | |
| | | | | | Signal Peptide: M1-S20 | HMMER |
| | | | | | EGF-like domain: C295-C334, C100-C135, C254-C289, | HMME_RPFAM |
| | | | | | C57-C94 | |
| | | | | | TB domain Y163-L205 | HMMER_PFAM |
| | | | | | Calcium-binding EGF-like domain proteins pattern | BLIMPS_BLOCKS |
| | | | | | proteins BL01187: C94-S105, C310-Y325 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD028384: C206-G259 | |
| | | | | | TGFBP REPEAT DM00210\|P22064\|1188-1273: | BLAST_DOMO |
| | | | | | Q144-T230 DM00210\|Q00918\|1506-1591: Q144-Y229 | |
| | | | | | EGF DM00003 | BLAST_DOMO |
| | | | | | \|P22064\|1336-1383: V292-A340 | |
| | | | | | \|P22064\|1139-1186: F95-E143 | |
| | | | | | EGF-like domain signature 2: C274-C289, C319-C334 | MOTIFS |
| | | | | | Calcium-binding EGF-like domain pattern signature: | MOTIFS |
| | | | | | D53-C79, D96-C120, D291-C319 | |
| | | | | | Aspartic acid and asparagine hydroxylation site: C70-C81, | MOTIFS |
| | | | | | C111-C122, C310-C321 | |
| 4 | 7500665CD1 | 381 | S23 S74 S104 S106 | | signal_cleavage: M1-A27 | SPSCAN |
| | | | S139 S296 S297 | | | |
| | | | S319 S330 T227 | | | |
| | | | T261 T323 Y226 | | | |
| | | | | | Signal Peptide: M1-A27, M1-G24 | HMMER |
| | | | | | Granin (chromogranin or secretogranin): M1-M378 | HMMER_PFAM |
| | | | | | Cytosolic domain: M1-T6 | TMHMMER |
| | | | | | Transmembrane domain: H7-S29 | |
| | | | | | Non-cytosolic domain: F30-M381 | |
| | | | | | Granins proteins BL00422: L35-E63, Y78-P87, | BLIMPS_BLOCKS |
| | | | | | D220-G247 | |
| | | | | | CHROMOGRANIN PRECURSOR SIGNAL | BLAST_PRODOM |
| | | | | | CALCIUM BINDING A CONTAINS: CGA | |
| | | | | | PANCREASTATIN WE14 AMIDATION | |
| | | | | | PD012346: P51-G318 | |
| | | | | | SECRETOGRANIN II PRECURSOR SGII | BLAST_PRODOM |
| | | | | | CHROMOGRANIN C SULFATATION | |
| | | | | | CLEAVAGE ON PAIR PD014505: M1-R43 | |
| | | | | | GRANINS DM07917 | BLAST_DOMO |
| | | | | | \|P20616\|1-612: M1-Q306, E281-M381 | |
| | | | | | \|P10362\|1-618: M1-E304, E281-M381 | |
| 5 | 3569792CD1 | 991 | S3 S52 S302 S419 | N417 N683 | signal_cleavage: M1-A20 | SPSCAN |
| | | | S469 S481 S487 | N754 N783 | | |
| | | | S528 S529 S581 | | | |
| | | | S622 S737 S851 | | | |
| | | | S865 S889 S903 | | | |
| | | | T49 T96 T175 | | | |
| | | | T211 T235 T274 | | | |
| | | | T424 T439 T657 | | | |
| | | | T729 T730 T784 | | | |
| | | | T786 T871 T886 | | | |
| | | | T913 T964 | | | |
| | | | | | Signal Peptide: M1-A18, M1-A20, M1-Q22, M1-A26 | HMMER |
| | | | | | CUB domain: C802-Y911 | HMMER_PFAM |
| | | | | | EGF-like domain: C33-C68, C281-C316, C116-C151, | HMMER_PFAM |
| | | | | | C240-C275, C361-C397, C74-C110, C161-C197, | |
| | | | | | C201-C236, C322-C355 | |
| | | | | | Calcium-binding EGF-like domain proteins pattern | BLIMPS_BLOCKS |
| | | | | | proteins BL01187: C110-G121, C372-Q387 | |
| | | | | | Thrombomodulin signature PR00907: C208-H224, | BLIMPS_PRINTS |
| | | | | | G337-S362 | |
| | | | | | GLYCOPROTEIN THYROGLOBULIN | BLAST_PRODOM |
| | | | | | PRECURSOR REPEAT THYROID HORMONE | |
| | | | | | IODINATION SIGNAL EGF-LIKE PROTEIN | |
| | | | | | PD009765: C634-C741, C650-C797 | |
| | | | | | EGF-LIKE DOMAIN DM00864\|I55476\|159-241: | BLAST_DOMO |
| | | | | | N285-C361, N244-D325, I45-E118, E77-C151, N205-R282 | |
| | | | | | EGF DM00003 | BLAST_DOMO |
| | | | | | \|P98163\|1373-1460: C281-L349, C82-C151, C236-I319 | |
| | | | | | \|P25723\|741-788: D277-F324 | |
| | | | | | \|P98063\|706-753: D112-C151 | |
| | | | | | Calcium-binding EGF-like domain pattern signature: | MOTIFS |
| | | | | | D29-C55, D70-C95, D112-C136, D277-C301, D318-C341, | |
| | | | | | D357-C381 | |
| | | | | | Aspartic acid and asparagine hydroxylation site: C46-C57, | MOTIFS |
| | | | | | C86-C97, C127-C138, C292-C303, C332-C343, | |
| | | | | | C372-C383 | |
| | | | | | EGF-like domain signature 2: C55-C68, C95-C110, | MOTIFS |
| | | | | | C136-C151, C182-C197, C260-C275, C301-C316, | |
| | | | | | C341-C355 | |
| 6 | 7500100CD1 | 306 | S98 S113 S188 | N110 | signal_cleavage: M1-A18 | SPSCAN |
| | | | S192 S220 S224 | | | |
| | | | S246 S247 S287 | | | |
| | | | T59 | | | |
| | | | | | Signal Peptide: M1-V16, M1-A18, M1-P20, M1-S23 | HMMER |
| | | | | | Granin (chromogranin or secretogranin): M1-G306 | HMMER_PFAM |
| | | | | | Granins proteins BL00422: L181-E204, E271-G306, | BLIMPS_BLOCKS |
| | | | | | L9-V37 | |
| | | | | | Granins signatures: G14-L76, P264-G306 | PROFILESCAN |
| | | | | | Chromogranin signature PR00659: N26-S41, S41-C56, | BLIMPS_PRINTS |
| | | | | | E279-A297 | |
| | | | | | CHROMOGRANIN PRECURSOR SIGNAL | BLAST_PRODOM |
| | | | | | CALCIUM-BINDING A CONTAINS: CGA | |
| | | | | | PANCREASTATIN WE14 AMIDATION | |
| | | | | | PD012346: S45-G306, M1-D280, 0262-G306 | |
| | | | | | CHROMOGRANIN A DM07723 | BLAST_DOMO |
| | | | | | \|P05059\|1-448: L108-G306, M1-G306 | |
| | | | | | \|P26339\|1-462: S23-G306, M1-A229 | |
| | | | | | ATP/GTP-binding site motif A (P-loop): G164-S171 | MOTIFS |
| | | | | | Granins signature 1: E284-L293 | MOTIFS |
| | | | | | Granins signature 2: C35-C56 | MOTIFS |
| 7 | 5201851CD1 | 1668 | S81 S88 S183 S253 | N347 N378 | signal_cleavage: M1-G23 | SPSCAN |
| | | | S254 S414 S501 | N424 N620 | | |
| | | | S576 S602 S647 | N1144 N1197 | | |
| | | | S685 S1001 S1047 | N1313 | | |
| | | | S1213 S1304 S1360 | | | |
| | | | S1423 S1449 S1563 | | | |
| | | | S1634 S1646 S1665 | | | |
| | | | T29 T84 T87 T272 | | | |
| | | | T349 T426 T651 | | | |
| | | | T722 T763 T938 | | | |
| | | | T954 T1134 T1146 | | | |
| | | | T1188 T1199 | | | |
| | | | T1278 T1280 | | | |
| | | | T1315 T1403 | | | |
| | | | T1493 T1529 | | | |
| | | | T1602 T1606 | | | |
| | | | | | Signal Peptide: L6-G23, M1-A18, M1-A21, M1-G23, | HMMER |
| | | | | | M1-L25, M1-S20, M1-R27 | |
| | | | | | EGF-like domain: C1613-C1652, C630-C665, | HMMER_PFAM |
| | | | | | C1418-C1453, C824-C860, C866-C902, C403-C430, | |
| | | | | | C1030-C1065, C1071-C1106, C1572-C1607, | |
| | | | | | C1195-C1231, C989-C1024, C1153-C1189, | |
| | | | | | C1237-C1274, C191-C218, | |
| | | | | | C1112-C1147, C1375-C1412, C908-C943, | |
| | | | | | C949-C983 | |
| | | | | | TB domain: Y1481-L1523, S1304-M1347, R687-V728, | HMMER_PFAM |
| | | | | | S566-M609 | |
| | | | | | Calcium-binding EGF-like domain proteins pattern | BLIMPS_BLOCKS |
| | | | | | proteins BL01187: C943-T954, C1628-Y1643 | |
| | | | | | Type II EGF-like signature PR00010: N980-D987, | BLIMPS_PRINTS |
| | | | | | G1129-F1139, W1327-I1333 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD077759: M1-G171 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: P718-E823 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD097076: E219-A341 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF BETA BETA PD007480: F398-P506 | |
| | | | | | LATENT; EGF; TRANSFORMING; GROWTH; | BLAST_DOMO |
| | | | | | DM06956\|P22064\|112-225: S438-A552 | |
| | | | | | DM06956\|Q00918\|430-543: S438-A552 | |
| | | | | | TGFBP REPEAT DM00210 | BLAST_DOMO |
| | | | | | \|P22064\|1188-1273: Q1462-T1548 | |
| | | | | | \|Q00918\|1506-1591: Q1462-Y1547 | |
| | | | | | Aspartic acid and asparagine hydroxylation site: | MOTIFS |
| | | | | | C641-C652, C836-C847, C878-C889, C1000-C1011, | |
| | | | | | C1041-C1052, C1082-C1093, C1124-C1135, | |
| | | | | | C1165-C1176, C1207-C1218, C1249-C1260, | |
| | | | | | C1388-C1399, C1429-C1440, C1628-C1639 | |
| | | | | | EGF-like domain signature 1: C207-C218, C419-C430 | MOTIFS |
| | | | | | EGF-like domain signature 2: C650-C665, C845-C860, | MOTIFS |
| | | | | | C887-C902, C1009-C1024, C1050-C1065, | |
| | | | | | C1091-C1106, C1133-C1147, C1174-C1189, | |
| | | | | | C1216-C1231, C1592-C1607, C1637-C1652 | |
| | | | | | Calcium-binding EGF-like domain pattern signature: | MOTIFS |
| | | | | | D626-C650, E820-C845, D862-C887, D904-C928, | |
| | | | | | D945-C969, D985-C1009, D1026-C1050, D1067-C1091, | |
| | | | | | D1108-C1133, D1149-C1174, D1191-C1216, | |
| | | | | | D1233-C1258, D1371-C1397, D1414-C1438, | |
| | | | | | D1609-C1637 | |
| 8 | 7500667CD1 | 504 | S23 S186 S210 | N263 | signal_cleavage: M1-A27 | SPSCAN |
| | | | S282 S319 S419 | | | |
| | | | S420 S442 S453 | | | |
| | | | T114 T148 T446 | | | |
| | | | Y113 Y394 | | | |
| | | | | | Signal Peptide: G10-A27, M1-A27, M1-G24 | HMMER |
| | | | | | Granin (chromogranin or secretogranin): M1-M501 | HMMER_PFAM |
| | | | | | Cytosolic domain: M1-T6Transmembrane domain: | TMHMMER |
| | | | | | H7-S29Non-cytosolic domain: F30-M504 | |
| | | | | | Granins proteins BL00422: L35-E63, D107-G134, | BLIMPS_BLOCKS |
| | | | | | G216-D251, Q174-V197 | |
| | | | | | Granins signatures: S362-V411 | PROFILESCAN |
| | | | | | CHROMOGRANIN PRECURSOR SIGNAL | BLAST_PRODOM |
| | | | | | CALCIUM BINDING A CONTAINS: CGA | |
| | | | | | PANCREASTATIN WE14 AMIDATION | |
| | | | | | PD012346: E63-M501 | |
| | | | | | SECRETOGRANIN II PRECURSOR SGII | BLAST_PRODOM |
| | | | | | CHROMOGRANIN C SULFATATION | |
| | | | | | CLEAVAGE ON PAIR PD014505: M1-R43 | |
| | | | | | GRANINS DM07917\|P20616\|1-612: E63-M504, M1-K498 | BLAST_DOMO |
| | | | | | DM07917\|P10362\|1-618: Q37-M504, M1-E60 | |
| | | | | | Granins signature 1: E382-L391 | MOTIFS |
| 9 | 7744055CD1 | 317 | S82 S84 S96 S113 | | signal_cleavage: M1-A44 | SPSCAN |
| | | | S117 S121 S181 | | | |
| | | | S187 S235 S245 | | | |
| | | | S265 T111 T115 | | | |
| | | | T123 T127 T135 | | | |
| | | | T139 T160 T225 | | | |
| | | | T309 | | | |
| | | | | | Signal Peptide: I24-A52, M1-A46 | HMMER |
| | | | | | G-protein alpha subunit: G261-E285 | HMMER_PFAM |
| | | | | | NEUROENDOCRINE SECRETORY PROTEIN 55 | BLAST_PRODOM |
| | | | | | PD069414: E130-P241 | |
| | | | | | NEUROENDOCRINE SECRETORY PROTEIN 55 | BLAST_PRODOM |
| | | | | | PD069627: M1-Y109 | |
| | | | | | GTP-BINDING REGULATORY PROTEIN GS | BLAST_DOMO |
| | | | | | ALPHA CHAIN DM00104 | |
| | | | | | \|S10508\|7-149: G261-A298 | |
| | | | | | \|S52418\|459-601: G261-A298 | |
| | | | | | \|P16052\|7-149: G261-A298 | |
| | | | | | \|S34421\|32-174: A262-A298 | |
| | | | | | ATP/GTP-binding site motif A (P-loop): G261-S268 | MOTIFS |
| 10 | 7502082CD1 | 1721 | S81 S88 S183 S253 | N347 N378 | signal_cleavage: M1-G23 | SPSCAN |
| | | | S254 S414 S501 | N424 N620 | | |
| | | | S576 S602 S647 | N1197 N1250 | | |
| | | | S685 S1054 S1100 | N1366 | | |
| | | | S1266 S1357 S1413 | | | |
| | | | S1476 S1502 S1616 | | | |
| | | | S1687 S1699 S1718 | | | |
| | | | T29 T84 T87 T272 | | | |
| | | | T349 T426 T651 | | | |
| | | | T816 T991 T1007 | | | |
| | | | T1187 T1199 | | | |
| | | | T1241 T1252 | | | |
| | | | T1331 T1333 | | | |
| | | | T1368 T1456 | | | |
| | | | T1546 T1582 | | | |
| | | | T1655 T1659 | | | |
| | | | | | Signal Peptide: L6-G23, M1-A18, M1-A21, M1-G23, | HMMER |
| | | | | | M1-L25, M1-R27, M1-S20 | |
| | | | | | EGF-like domain: C1666-C1705, C630-C665, | HMMER_PFAM |
| | | | | | C1471-C1506, C877-C913, C919-C955, C403-C430, | |
| | | | | | C1083-C1118, C1124-C1159, C1625-C1660, | |
| | | | | | C1248-C1284, C1042-C1077, C1206-C1242, | |
| | | | | | C1290-C1327, C191-C218, | |
| | | | | | C1165-C1200, C1428-C1465, C961-C996, | |
| | | | | | C1002-C1036 | |
| | | | | | TB domain: R687-I728, Y1534-L1576, S1357-M1400, | HMMER_PFAM |
| | | | | | S566-M609 | |
| | | | | | Calcium-binding EGF-like domain proteins pattern | BLIMPS_BLOCKS |
| | | | | | proteins BL01187: C996-T1007, C1681-Y1696 | |
| | | | | | Type II EGF-like signature PR00010: N1033-D1040, | BLIMPS_PRINTS |
| | | | | | G1182-F1192, W1380-I1386 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: C729-E876 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD077759: M1-G171 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD097076: E219-A341 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF BETA BETA PD007480: F398-P506 | |
| | | | | | LATENT; EGF; TRANSFORMING; GROWTH; | BLAST_DOMO |
| | | | | | DM06955\|P22064\|418-542: P745-Q870 | |
| | | | | | DM06955\|Q00918\|737-861: P745-Q870 | |
| | | | | | DM06956\|P22064\|112-225: S438-A552 | |
| | | | | | DM06956\|Q00918\|430-543: S438-A552 | |
| | | | | | Aspartic acid and asparagine hydroxylation site: | MOTIFS |
| | | | | | C641-C652, C889-C900, C931-C942, C1053-C1064, | |
| | | | | | C1094-C1105, C1135-C1146, C1177-C1188, | |
| | | | | | C1218-C1229, C1260-C1271, C1302-C1313, | |
| | | | | | C1441-C1452, C1482-C1493, C1681-C1692 | |
| | | | | | EGF-like domain signature 1: C207-C218, C419-C430 | MOTIFS |
| | | | | | EGF-like domain signature 2: C650-C665, C898-C913, | MOTIFS |
| | | | | | C940-C955, C1062-C1077, C1103-C1118, | |
| | | | | | C1144-C1159, C1186-C1200, C1227-C1242, | |
| | | | | | C1269-C1284, C1645-C1660, C1690-C1705 | |
| | | | | | Calcium-binding EGF-like domain pattern signature: | MOTIFS |
| | | | | | D626-C650, E873-C898, D915-C940, D957-C981, | |
| | | | | | D998-C1022, D1038-C1062, D1079-C1103, D1120-C1144, | |
| | | | | | D1161-C1186, D1202-C1227, D1244-C1269, | |
| | | | | | D1286-C1311, D1424-C1450, D1467-C1491, | |
| | | | | | D1662-C1690 | |
| 11 | 7502084CD1 | 1679 | S81 S88 S183 S253 | N347 N378 | signal_cleavage: M1-G23 | SPSCAN |
| | | | S254 S414 S501 | N424 N620 | | |
| | | | S576 S602 S647 | N1197 N1324 | | |
| | | | S685 S1054 S1100 | | | |
| | | | S1315 S1371 S1434 | | | |
| | | | S1460 S1574 S1645 | | | |
| | | | S1657 S1676 T29 | | | |
| | | | T84 T87 T272 | | | |
| | | | T349 T426 T651 | | | |
| | | | T816 T991 T1007 | | | |
| | | | T1187 T1199 | | | |
| | | | T1241 T1289 | | | |
| | | | T1291 T1326 | | | |
| | | | T1414 T1504 | | | |
| | | | T1540 T1613 | | | |
| | | | T1617 | | | |
| | | | | | Signal Peptide: L6-G23, M1-A18, M1-A21, M1-G23, | HMMER |
| | | | | | M1-L25, M1-R27, M1-S20 | |
| | | | | | EGF-like domain: C1624-C1663, C630-C665, | HMMER_PFAM |
| | | | | | C1429-C1464, C877-C913, C919-C955, | |
| | | | | | C403-C430, C1083-C1118, C1124-C1159, | |
| | | | | | C1583-C1618, C1042-C1077, C1206-C1242, | |
| | | | | | C1248-C1285, C191-C218, C1165-C1200, | |
| | | | | | C1386-C1423, C961-C996, C1002-C1036 | |
| | | | | | TB domain: R687-I728, Y1492-L1534, S1315-M1358, | HMMER_PFAM |
| | | | | | S566-M609 | |
| | | | | | Calcium-binding EGF-like domain proteins pattern | BLIMPS_BLOCKS |
| | | | | | proteins BL01187: C996-T1007, C1639-Y1654 | |
| | | | | | Type II EGF-like signature PR00010: N1033-D1040, | BLIMPS_PRINTS |
| | | | | | G1182-F1192, W1338-I1344 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: C729-E876 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD077759: M1-G171 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD097076: E219-A341 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF BETA BETA PD007480: F398-P506 | |
| | | | | | LATENT; EGF; TRANSFORMING; GROWTH; | BLAST_DOMO |
| | | | | | DM06955\|P22064\|418-542: P745-Q870 | |
| | | | | | DM06955\|Q00918\|737-861: P745-Q870 | |
| | | | | | DM06956\|P22064\|112-225: S438-A552 | |
| | | | | | DM06956\|Q00918\|430-543: 5438-A552 | |
| | | | | | Aspartic acid and asparagine hydroxylation site: | MOTIFS |
| | | | | | C641-C652, C889-C900, C931-C942, C1053-C1064, | |
| | | | | | C1094-C1105, C1135-C1146, C1177-C1188, | |
| | | | | | C1218-C1229, C1260-C1271, C1399-C1410, | |
| | | | | | C1440-C1451, C1639-C1650 | |
| | | | | | EGF-like domain signature 1: C207-C218, C419-C430 | MOTIFS |
| | | | | | EGF-like domain signature 2: C650-C665, C898-C913, | MOTIFS |
| | | | | | C940-C955, C1062-C1077, C1103-C1118, | |
| | | | | | C1144-C1159, C1186-C1200, C1227-C1242, | |
| | | | | | C1603-C1618, C1648-C1663 | |
| | | | | | Calcium-binding EGF-like domain pattern signature: | MOTIFS |
| | | | | | D626-C650, E873-C898, D915-C940, D957-C981, | |
| | | | | | D998-C1022, D1038-C1062, D1079-C1103, D1120-C1144, | |
| | | | | | D1161-C1186, D1202-C1227, D1244-C1269, | |
| | | | | | D1382-C1408, D1425-C1449, D1620-C1648 | |
| 12 | 7502085CD1 | 1626 | S81 S88 S183 S253 | N347 N378 | signal_cleavage: M1-G23 | SPSCAN |
| | | | S254 S414 S501 | N424 N620 | | |
| | | | S576 S602 S647 | N1144 N1271 | | |
| | | | S685 S1001 S1047 | | | |
| | | | S1262 S1318 S1381 | | | |
| | | | S1407 S1521 S1592 | | | |
| | | | S1604 S1623 T129 | | | |
| | | | T84 T87 T272 | | | |
| | | | T349 T426 T651 | | | |
| | | | T722 T763 T938 | | | |
| | | | T954 T1134 T1146 | | | |
| | | | T1188 T1236 | | | |
| | | | T1238 T1273 | | | |
| | | | T1361 T1451 | | | |
| | | | T1487 T1560 | | | |
| | | | T1564 | | | |
| | | | | | Signal Peptide: L6-G23, M1-A18, M1-A21, M1-G23, | HMMER |
| | | | | | M1-L25, M1-R27, M1-S20 | |
| | | | | | EGF-like domain: C1571-C1610, C630-C665, | HMMER_PFAM |
| | | | | | C1376-C1411, C824-C860, C866-C902, | |
| | | | | | C403-C430, C1030-C1065, | |
| | | | | | C1071-C1106, C1530-C1565, C989-C1024, | |
| | | | | | C1153-C1189, C1195-C1232, C191-C218, C1112-C1147, | |
| | | | | | C1333-C1370, C908-C943, C949-C983 | |
| | | | | | TB domain: Y1439-L1481, S1262-M1305, R687-V728, | HMMER_PFAM |
| | | | | | S566-M609 | |
| | | | | | Calcium-binding EGF-like domain proteins pattern | BLIMPS_BLOCKS |
| | | | | | proteins BL01187: C943-T954, C1586-Y1601 | |
| | | | | | Type II EGF-like signature PR00010: N980-D987, | BLIMPS_PRINTS |
| | | | | | G1129-F1139, W1285-I1291 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD077759: M1-G171 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: P718-E823 | |
| | | | | | PROTEIN LATENT BETA BINDING EGF-LIKE | BLAST_PRODOM |
| | | | | | DOMAIN TGF GLYCOPROTEIN | |
| | | | | | TRANSFORMING GROWTH PD097076: E219-A341 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF BETA BETA PD007480: F398-P506 | |
| | | | | | LATENT; EGF; TRANSFORMING; GROWTH | BLAST_DOMO |
| | | | | | DM06956 | |
| | | | | | \|P22064\|112-225: S438-A552 | |
| | | | | | \|Q00918\|430-543: S438-A552 | |
| | | | | | TGFBP REPEAT DM00210\|P22064\|1118-1273: | BLAST_DOMO |
| | | | | | Q1420-T1506 DM00210\|Q00918\|1506-1591: Q1420-Y1505 | |
| | | | | | Aspartic acid and asparagine hydroxylation site: | MOTIFS |
| | | | | | C641-0652, C836-0847, C878-C889, C1000-C1011, | |
| | | | | | C1041-C1052, C1082-C1093, C1124-C1135, | |
| | | | | | C1165-C1176, C1207-C1218, C1346-C1357, | |
| | | | | | C1387-C1398, C1586-C1597 | |
| | | | | | EGF-like domain signature 1: C207-C218, C419-C430 | MOTIFS |
| | | | | | EGF-like domain signature 2: C650-C665, C845-C860, | MOTIFS |
| | | | | | C887-C902, C1009-C1024, C1050-C1065, | |
| | | | | | C1091-C1106, C1133-C1147, C1174-C1189, | |
| | | | | | C1550-C1565, C1595-C1610 | |
| | | | | | Calcium-binding EGF-like domain pattern signature: | MOTIFS |
| | | | | | D626-C650, E820-C845, D862-C887, D904-C928, | |
| | | | | | D945-C969, D985-C1009, D1026-C1050, D1067-C1091, | |
| | | | | | D1108-C1133, D1149-C1174, D1191-C1216, | |
| | | | | | D1329-C1355, D1372-C1396, D1567-C1595 | |
| 13 | 7502093CD1 | 1300 | S88 S175 S250 | N21 N52 N98 | signal_cleavage: M1-S20 | SPSCAN |
| | | | S276 S321 S359 | N294 N818 | | |
| | | | S675 S721 S936 | N945 | | |
| | | | S992 S1055 S1081 | | | |
| | | | S1195 S1266 S1278 | | | |
| | | | S1297 T23 T100 | | | |
| | | | T325 T396 T437 | | | |
| | | | T612 T628 T808 | | | |
| | | | T820 T862 T910 | | | |
| | | | T912 T947 T1035 | | | |
| | | | T1125 T1161 | | | |
| | | | T1234 T1238 | | | |
| | | | | | Signal Peptide: M1-S20 | HMMER |
| | | | | | EGF-like domain: C1245-C1284, C304-C339, | HMMER_PFAM |
| | | | | | C1050-C1085, C498-C534, C540-C576, | |
| | | | | | C77-C104, C704-C739, C745-C780, | |
| | | | | | C1204-C1239, C663-C698, C827-C863, | |
| | | | | | C869-C906, C786-C821, C1007-C1044, C582-C617, | |
| | | | | | C623-C657 | |
| | | | | | TB domain: Y1113-L1155, S936-M979, R361-V402, | HMMER_PFAM |
| | | | | | S240-M283 | |
| | | | | | Calcium-binding EGF-like domain proteins pattern | BLIMPS_BLOCKS |
| | | | | | proteins BL01187: C617-T628, C1260-Y1275 | |
| | | | | | Type II EGF-like signature PR00010: N654-D661, | BLIMPS_PRINTS |
| | | | | | G803-F813, W959-I965 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: P392-E497 | |
| | | | | | LATENT BINDING EGE-LIKE DOMAIN | BLAST_PRODOM |
| | | | | | PROTEIN GLYCOPROTEIN TRANSFORMING | |
| | | | | | GROWTH TGF-BETA BETA PD007480: F72-P180 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD034912: G182-S240 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD028384: C1156-G1209 | |
| | | | | | LATENT; EGF; TRANSFORMING; GROWTH; | BLAST_DOMO |
| | | | | | DM06956\|P22064\|112-225: S112-A226 | |
| | | | | | LATENT; EGF; TRANSFORMING; GROWTH; | BLAST_DOMO |
| | | | | | DM06956\|Q00918\|430-543: S112-A226 | |
| | | | | | TGFBP REPEAT DM00210\|P22064\|1188-1273: | BLAST_DOMO |
| | | | | | Q1094-T1180 | |
| | | | | | TGFBP REPEAT DM00210\|Q00918\|1506-1591: | BLAST_DOMO |
| | | | | | Q1094-Y1179 | |
| | | | | | Aspartic acid and asparagine hydroxylation site: | MOTIFS |
| | | | | | C315-C326, C510-C521, C552-C563, | |
| | | | | | C674-C685, C715-C726, C756-C767, | |
| | | | | | C798-C809, C839-C850, C881-C892, | |
| | | | | | C1020-C1031, C1061-C1072, C1260-C1271 | |
| | | | | | EGF-like domain signature 1: C93-C104 | MOTIFS |
| | | | | | EGF-like domain signature 2: C324-C339, C519-C534, | MOTIFS |
| | | | | | C561-C576, C683-C698, C724-C739, C765-C780, | |
| | | | | | C807-C821, C848-C863, C1224-C1239, | |
| | | | | | C1269-C1284 | |
| | | | | | Calcium-binding EGF-like domain pattern signature: | MOTIFS |
| | | | | | D300-C324, E494-C519, D536-C561, D578-C602, | |
| | | | | | D619-C643, D659-C683, D700-C724, D741-C765, | |
| | | | | | D782-C807, D823-C848, D865-C890, D1003-C1029, | |
| | | | | | D1046-C1070, D1241-C1269 | |
| 14 | 7502097CD1 | 1353 | S88 S175 S250 | N21 N52 N98 | signal_cleavage: M1-S20 | SPSCAN |
| | | | S276 S321 S359 | N294 N871 | | |
| | | | S728 S774 S989 | N998 | | |
| | | | S1045 S1108 S1134 | | | |
| | | | S1248 S1319 S1331 | | | |
| | | | S1350 T23 T100 | | | |
| | | | T325 T490 T665 | | | |
| | | | T681 T861 T873 | | | |
| | | | T915 T963 T965 | | | |
| | | | T1000 T1088 | | | |
| | | | T1178 T1214 | | | |
| | | | T1287 T1291 | | | |
| | | | | | Signal Peptide: M1-S20 | HMMER |
| | | | | | EGF-like domain: C1298-C1337, C304-C339, | HMMER_PFAM |
| | | | | | C1103-C1138, C551-C587, C593-C629, C77-C104, | |
| | | | | | C757-C792, C798-C833, C1257-C1292, C716-C751, | |
| | | | | | C880-C916, C922-C959, C839-C874, C1060-C1097, | |
| | | | | | C635-C670, C676-C710 | |
| | | | | | TB domain: P361-I402, Y1166-L1208, S989-M1032, | HMMER_PFAM |
| | | | | | S240-M283 | |
| | | | | | Calcium-binding EGF-like domain proteins pattern | BLIMPS_BLOCKS |
| | | | | | proteins BL01187: C670-T681, C1313-Y1328 | |
| | | | | | Type II EGF-like signature PR00010: N707-D714, | BLIMPS_PRINTS |
| | | | | | G856-F866, W1012-I1018 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: C403-E550 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF BETA BETA PD007480: F72-P180 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD034912: G182-S240 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD028384: C1209-G1262 | |
| | | | | | LATENT; EGF; TRANSFORMING; GROWTH; | BLAST_DOMO |
| | | | | | DM06955\|P22064\|418-542: P419-Q544 | |
| | | | | | LATENT; EGF; TRANSFORMING; GROWTH; | BLAST_DOMO |
| | | | | | DM06955\|Q00918\|737-861: P419-Q544 | |
| | | | | | LATENT; EGF; TRANSFORMING; GROWTH; | BLAST_DOMO |
| | | | | | DM06956\|P22064\|112-225: S112-A226 | |
| | | | | | LATENT; EGF; TRANSFORMING; GROWTH; | BLAST_DOMO |
| | | | | | DM06956\|Q00918\|430-543: S112-A226 | |
| | | | | | Aspartic acid and asparagine hydroxylation site: | MOTIFS |
| | | | | | C315-C326, C563-C574, C605-C616, | |
| | | | | | C727-C738, C768-C779, C809-C820, | |
| | | | | | C851-C862, C892-C903, C934-C945, | |
| | | | | | C1073-C1084, C1114-C1125, C1313-C1324 | |
| | | | | | EGF-like domain signature 1: C93-C104 | MOTIFS |
| | | | | | EGF-like domain signature 2: C324-C339, C572-C587, | MOTIFS |
| | | | | | C614-C629, C736-C751, C777-C792, C818-C833, | |
| | | | | | C860-C874, C901-C916, C1277-C1292, | |
| | | | | | C1322-C1337 | |
| | | | | | Calcium-binding EGF-like domain pattern signature: | MOTIFS |
| | | | | | D300-C324, E547-C572, D589-C614, D631-C655, | |
| | | | | | D672-C696, D712-C736, D753-C777, D794-C818, | |
| | | | | | D835-C860, D876-C901, D918-C943, D1056-C1082, | |
| | | | | | D1099-C1123, D1294-C1322 | |
| 15 | 7502108CD1 | 1342 | S88 S175 S250 | N21 N52 N98 | signal_cleavage: M1-S20 | SPSCAN |
| | | | S276 S321 S359 | N294 N818 | | |
| | | | S675 S721 S887 | N871 N987 | | |
| | | | S978 S1034 S1097 | | | |
| | | | S1123 S1237 S1308 | | | |
| | | | S1320 S1339 T23 | | | |
| | | | T100 T325 T396 | | | |
| | | | T437 T612 T628 | | | |
| | | | T808 T820 T862 | | | |
| | | | T873 T952 T954 | | | |
| | | | T989 T1077 T1167 | | | |
| | | | T1203 T1276 | | | |
| | | | T1280 | | | |
| | | | | | Signal Peptide: M1-S20 | HMMER |
| | | | | | EGF-like domain: C1287-C1326, C304-C339, | HMMER_PFAM |
| | | | | | C1092-C1127, C498-C534, C540-C576, | |
| | | | | | C77-C104, C704-C739, C745-C780, | |
| | | | | | C1246-C1281, C869-C905, C663-C698, | |
| | | | | | C827-C863, C911-C948, C786-C821, C1049-C1086, | |
| | | | | | C582-C617, C623-C657 | |
| | | | | | TB domain: Y1155-L1197, S978-M1021, R361-V402, | HMMER_PFAM |
| | | | | | S240-M283 | |
| | | | | | Calcium-binding EGF-like domain proteins pattern | BLIMPS_BLOCKS |
| | | | | | proteins BL01187: C617-T628, C1302-Y1317 | |
| | | | | | Type II EGF-like signature PR00010: N654-D661, | BLIMPS_PRINTS |
| | | | | | G803-F813, W1001-I1007 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD033821: P392-E497 | |
| | | | | | LATENT BINDING EGF-LIKE DOMAIN | BLAST_PRODOM |
| | | | | | PROTEIN GLYCOPROTEIN TRANSFORMING | |
| | | | | | GROWTH TGF-BETA BETA PD007480: F72-P180 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD034912: G182-S240 | |
| | | | | | PROTEIN LATENT BETA BINDING EGF-LIKE | BLAST_PRODOM |
| | | | | | DOMAIN TRANSFORMING GROWTH FACTOR | |
| | | | | | PRECURSOR PD028384: C1198-G1251 | |
| | | | | | LATENT; EGF; TRANSFORMING; GROWTH; | BLAST_DOMO |
| | | | | | DM06956\|P22064\|112-225: S112-A226 | |
| | | | | | LATENT; EGF; TRANSFORMING; GROWTH; | BLAST_DOMO |
| | | | | | DM06956\|Q00918\|430-543: S112-A226 | |
| | | | | | TGFBP REPEAT DM00210\|P22064\|1188-1273: | BLAST_DOMO |
| | | | | | Q1136-T1222 | |
| | | | | | TGFBP REPEAT DM00210\|Q00918\|1506-1591: | BLAST_DOMO |
| | | | | | Q1136-Y1221 | |
| | | | | | Aspartic acid and asparagine hydroxylation site: | MOTIFS |
| | | | | | C315-C326, C510-C521, C552-C563, | |
| | | | | | C674-C685, C715-C726, C756-C767, | |
| | | | | | C798-C809, C839-C850, C881-C892, | |
| | | | | | C923-C934, C1062-C1073, C1103-C1114, | |
| | | | | | C1302-C1313 | |
| | | | | | EGF-like domain signature 1: C93-C104 | MOTIFS |
| | | | | | EGF-like domain signature 2: C324-C339, C519-C534, | MOTIFS |
| | | | | | C561-C576, C683-C698, C724-C739, C765-C780, | |
| | | | | | C807-C821, C848-C863, C890-C905, C1266-C1281, | |
| | | | | | C1311-C1326 | |
| | | | | | Calcium-binding EGF-like domain pattern signature: | MOTIFS |
| | | | | | D300-C324, E494-C519, D536-C561, D578-C602, | |
| | | | | | D619-C643, D659-C683, D700-C724, D741-C765, | |
| | | | | | D782-C807, D823-C848, D865-C890, D907-C932, | |
| | | | | | D1045-C1071, D1088-C1112, D1283-C1311 | |
| 16 | 7500668CD1 | 98 | S23 S74 | | Signal_cleavage: M1-A27 | SPSCAN |
| | | | | | Signal Peptide: G10-A27, M1-A27, M1-G24 | HMMER |
| | | | | | SECRETOGRANIN II PRECURSOR SGII | BLAST_PRODOM |
| | | | | | CHROMOGRANIN C SULFATATION | |
| | | | | | CLEAVAGE ON PAIR PD014505: M1-R43 | |
| | | | | | GRANINS DM07917\|P20616\|1-612: M1-S74 | BLAST_DOMO |
| | | | | | GRANINS DM07917\|P10362\|1-618: M1-S74 | BLAST_DOMO |
| 17 | 7505114CD1 | 133 | S5 S25 S51 S93 | N97 | Signal_cleavage: M1-C27 | SPSCAN |
| | | | S99 | | | |
| | | | | | Signal Peptide: M1-S25 | HMMER |
| | | | | | Interleukin 7/9 family: D28-G129 | HMMER_PFAM |
| | | | | | Interleukin-7 and -9 proteins BL00255: M1-M42, | BLIMPS_BLOCKS |
| | | | | | G56-L100, N107-G129 | |
| | | | | | Interleukin-7 signature PR00435: F2-S25, D26-L48, | BLIMPS_PRINTS |
| | | | | | S57-V77 | |
| | | | | | INTERLEUKIN7 PRECURSOR IL7 CYTOKINE | BLAST_PRODOM |
| | | | | | GROWTH FACTOR GLYCOPROTEIN SIGNAL 3- | |
| | | | | | D STRUCTURE PD013168: M1-T130 | |
| | | | | | INTERLEUKIN-7 DM07444\|P26895\|1-175: M1-H133 | BLAST_DOMO |
| | | | | | INTERLEUKIN-7 DM07444\|P10168\|1-153: M1-E132 | BLAST_DOMO |
| | | | | | Interleukin-7 and -9 signature: N107-L116 | MOTIFS |
| 18 | 7506452CD1 | 167 | S66 S147 T42 T73 | N59 | Signal_cleavage: M1-C23 | SPSCAN |
| | | | T93 | | | |
| | | | | | Signal Peptide: W8-S25, M1-C23, M1-A27, M1-P28 | HMMER |
| | | | | | Somatotropin hormone family: V105-C167, L12-N104 | HMMER_PFAM |
| | | | | | Somatotropin, prolactin and related hormones proteins | BLIMPS_BLOCKS |
| | | | | | BL00266: L46-Y72, Y137-R160 | |
| | | | | | Somatotropin, prolactin and related hormones | PROFILESCAN |
| | | | | | signatures: P122-C167 | |
| | | | | | Somatotropin hormone family signature PR00836: | BLIMPS_PRINTS |
| | | | | | C86-Q99, E130-D146, D146-R160 | |
| | | | | | HORMONE PRECURSOR SIGNAL PITUITARY | BLAST_PRODOM |
| | | | | | GROWTH SOMATOTROPIN PROLACTIN | |
| | | | | | GLYCOPROTEIN PRL PROTEIN PD000259: S11-K158, | |
| | | | | | E98-C167 | |
| | | | | | SOMATOTROPIN, PROLACTIN AND RELATED | BLAST_DOMO |
| | | | | | HORMONES | |
| | | | | | DM00125\|P01236\|28-223: P28-N164 | |
| | | | | | DM00125\|P33089\|1-195: L29-N164 | |
| | | | | | DM00125\|P55151\|28-223: P28-N164 | |
| | | | | | DM00125\|A61402\|29-224: P28-N164 | |
| | | | | | Somatotropin, prolactin and related hormones | MOTIFS |
| | | | | | signature 2: C142-C159 | |
| 19 | 7506730CD1 | 142 | S54 S93 S125 S139 | N62 | Signal Peptide: M1-G22 | HMMER |
| 20 | 7505046CD1 | 212 | S55 S75 T28 T96 | N74 | Signal_cleavage: M1-S23 | SPSCAN |
| | | | T121 T158 | | | |
| | | | | | Signal Peptide: M3-V20, M3-S23, M1-S23, M3-T29, | HMMER |
| | | | | | M3-C27 | |
| | | | | | Cytosolic domain: M1-Q6; Transmembrane domain: | TMHMMER |
| | | | | | R7-T29; Non-cytosolic domain: L30-G212 | |
| | | | | | TGF-BETA FAMILY DM00245\|P16047\|51-412: L51-I105 | BLAST_DOMO |
| 21 | 7506453CD1 | 75 | S55 S60 S64 | | signal_cleavage: M1-C23 | SPSCAN |
| | | | | | Signal Peptide: M1-C23, M1-S25, M1-A27, M1-P28 | HMMER |
| | | | | | Uteroglobin signature PR00486: K9-C23 | BLIMPS_PRINTS |
| 22 | 7509967CD1 | 173 | S66 S118 S163 T42 | N59 | signal_cleavage: M1-C23 | SPSCAN |
| | | | T73 T93 T151 | | | |
| | | | T170 Y124 | | | |
| | | | | | Signal Peptide: M1-C23, M1-S25, M1-A27, | HMMER |
| | | | | | M1-P28 | |
| | | | | | Somatotropin hormone family: S11-Y173 | HMMER_PFAM |
| | | | | | Somatotropin, prolactin and related hormones proteins | BLIMPS_BLOCKS |
| | | | | | BL00266: L46-Y72, C86-L123, E146-V162 | |
| | | | | | Uteroglobin family proteins BL00403: L13-A50 | BLIMPS_BLOCKS |
| | | | | | Somatotropin, prolactin and related hormones | PROFILESCAN |
| | | | | | signatures: E95-K143 | |
| | | | | | Somatotropin hormone family signature | BLIMPS_PRINTS |
| | | | | | PR00836: C86-Q99, F108-L126 | |
| | | | | | HORMONE PRECURSOR SIGNAL PITUITARY | BLAST_PRODOM |
| | | | | | GROWTH SOMATOTROPIN PROLACTIN | |
| | | | | | GLYCOPROTEIN PRL PROTEIN | |
| | | | | | PD000259: S11-E166 | |
| | | | | | SOMATOTROPIN, PROLACTIN AND RELATED | BLAST_DOMO |
| | | | | | HORMONES DM00125 | |
| | | | | | \|P01236\|28-223: P28-L165 | |
| | | | | | \|P55151\|28-223: P28-L165 | |
| | | | | | \|A61402\|29-224: P28-L165 | |
| | | | | | \|P33089\|1-195: L29-L165 | |
| | | | | | Somatotropin, prolactin and related hormones | MOTIFS |
| | | | | | signature1: C86-W119 | |

TABLE 4

| Polynucleotide SEQ | |
|---|---|
| ID NO:/Incyte ID/Sequence | |
| Length | Sequence Fragments |
| 23/7497502CB1/2598 | 1-711, 131-610, 131-653, 131-774, 134-749, 142-749, 272-818, 353-1036, 377-601, 377-905, 377-957, 377-961, 379-1081, |
| | 384-694, 386-957, 386-1037, 386-1093, 386-1118, 386-1130, 386-1137, 386-1183, 386-2598, 388-825, 390-1043, |
| | 393-911, 397-1006, 512-905, 519-1121, 576-872, 593-1180, 667-1330, 668-974, 707-1292, 714-1031, 714-1270, |
| | 768-1343, 834-1320, 839-870, 858-1533, 944-1271, 1209-1646, 1289-1802, 1369-1389, 1380-1648, 1557-2197, |
| | 1585-2163, 1586-2164, 1594-2225, 1607-2226, 1625-1656, 1625-1683, 1630-1650, 1632-2290, 1633-2096, |
| | 1640-2117, 1646-1919, 1650-2255, 1661-2151, 1669-1995, 1687-2254, 1690-2254, 1704-2278, 1718-2069, 1738-2033, |
| | 1775-2339, 1794-2036, 1796-2076, 1806-2287, 1807-2429, 1871-2157, 1919-2191, 1946-2238, 1994-2153 |
| 24/7103532CB1/2914 | 1-287, 64-511, 143-789, 144-476, 497-1242, 513-956, 550-827, 550-934, 563-1575, 612-889, 673-934, 766-934, 797-1053, |
| | 909-1174, 909-1432, 909-1549, 995-1352, 995-1476, 995-1541, 1030-1472, 1085-1324, 1122-1361, 1382-1882, |
| | 1439-1784, 1519-2018, 1519-2030, 1519-2060, 1543-1896, 1556-1933, 1567-1841, 1623-2074, 1623-2623, |
| | 1639-2021, 1651-1963, 1662-2340, 1662-2352, 1662-2424, 1663-2322, 1664-2487, 1687-2521, 1715-1947, 1717-2553, |
| | 1734-2549, 1742-2551, 1752-2160, 1752-2343, 1766-2503, 1772-2634, 1787-2545, 1790-2645, 1795-2551, |
| | 1797-2290, 1799-2543, 1829-2071, 1829-2830, 1859-2615, 1914-2551, 1920-2540, 1960-2723, 2013-2910, 2050-2844, |
| | 2052-2569, 2164-2746, 2168-2556, 2168-2579, 2224-2608, 2279-2537, 2299-2914, 2314-2914, 2346-2776, |
| | 2397-2532 |
| 25/7500108CB1/1458 | 1-642, 1-644, 1-684, 1-763, 1-772, 1-778, 1-839, 1-864, 1-901, 1-920, 200-369, 208-948, 210-392, 222-469, 222-475, |
| | 243-868, 275-488, 285-694, 309-440, 311-593, 312-566, 314-537, 324-576, 344-484, 371-551, 394-940, 407-692, |
| | 407-1036, 423-642, 423-1020, 436-1064, 449-698, 451-1073, 468-851, 494-654, 494-738, 503-791, 511-808, |
| | 511-1154, 511-1187, 512-1098, 514-830, 524-1075, 529-1362, 530-1359, 549-726, 554-691, 554-1129, 557-644, |
| | 568-1153, 568-1158, 568-1173, 568-1199, 568-1362, 587-841, 598-1362, 606-1362, 607-882, 608-1359, 613-872, |
| | 613-874, 615-1359, 640-1362, 647-934, 650-833, 650-1362, 651-1362, 666-1362, 679-889, 679-1260, 679-1362, |
| | 688-969, 688-995, 692-992, 703-1362, 705-1362, 724-999, 749-1012, 759-1362, 760-1154, 761-1049, 765-1362, |
| | 768-1359, 791-1048, 796-990, 796-1263, 806-1233, 806-1451, 810-1391, 811-1131, 816-1095, 824-1362, 854-1458, |
| | 858-1151, 867-1134, 876-1168, 898-1185, 900-1173, 952-1185, 973-1458 |
| 26/7500665CB1/1703 | 1-320, 1-564, 1-772, 12-394, 12-782, 14-253, 22-557, 22-674, 24-564, 25-718, 25-719, 25-1681, 30-532, 31-571, 33-271, |
| | 33-630, 33-699, 34-278, 34-292, 34-411, 34-477, 35-580, 37-286, 37-296, 37-358, 37-697, 37-783, 39-663, 39-877, |
| | 40-733, 41-300, 41-302, 41-329, 41-349, 41-551, 41-566, 41-583, 41-623, 41-679, 42-277, 42-391, 42-679, 42-694, |
| | 43-315, 43-850, 44-275, 46-694, 47-288, 47-689, 47-733, 48-682, 48-696, 49-854, 50-570, 55-527, 55-607, 57-321, |
| | 65-308, 67-350, 67-558, 67-684, 69-694, 69-697, 71-591, 71-676, 76-234, 79-772, 104-401, 104-744, 107-759, |
| | 122-358, 133-737, 150-814, 165-412, 165-447, 170-545, 171-676, 172-842, 175-780, 231-792, 245-794, 253-791, |
| | 295-553, 336-606, 346-761, 382- |
| | 893, 398-643, 398-665, 399-663, 423-960, 427-636, 458-716, 471-730, 476-931, 477-935, 489-932, 503-935, 505-724, |
| | 505-744, 506-939, 514-924, 550-818, 563-832, 573-872, 580-843, 580-844, 580-960, 583-852, 595-868, 637-937, |
| | 733-960, 744-960, 754-960, 809-1097, 951-1249, 955-1193, 955-1594, 955-1609, 958-1210, 961-1634, 962-1205, |
| | 964-1607, 976-1641, 979-1202, 979-1213, 979-1280, 979-1672, 982-1632, 983-1289, 984-1226, 986-1297, |
| | 999-1599, 999-1650, 1000-1227, 1000-1281, 1012-1629, 1028-1647, 1033-1328, 1034-1248, 1036-1283, 1046-1327, |
| | 1048-1293, 1057-1370, 1060-1535, 1069-1673, 1072-1538, |
| | 1077-1574, 1077-1672, 1086-1681, 1087-1288, 1097-1671, 1098-1400, 1116-1371, 1116-1572, 1120-1342, 1120-1346, |
| | 1120-1351, 1121-1676, 1121-1701, 1132-1391, 1153-1435, 1159-1426, 1160-1681, 1173-1681, 1181-1681, |
| | 1196-1639, 1196-1659, 1198-1680, 1199-1429, 1204-1676, 1204-1701, 1214-1462, 1215-1519, 1225-1470, 1229-1685, |
| | 1232-1681, 1238-1681, 1240-1472, 1249-1508, 1251-1674, 1251-1681, 1253-1680, 1256-1677, 1257-1681, |
| | 1263-1681, 1271-1680, 1279-1685, 1280-1682, 1298-1672, 1298-1685, 1299-1528, 1305-1694, 1312-1534, 1334-1545, |
| | 1338-1678, 1346-1596, 1347-1614, 1354-1573, 1356-1694, 1360-1681, 1364-1568, 1365-1672, 1367-1683, |
| | 1369-1603, 1374-1625, 1377-1605, 1379-1625, 1385-1609, 1386-1680, 1391-1699, 1392-1680, 1393-1521, 1409-1680, |
| | 1418-1638, 1426-1640, 1449-1600, 1450-1666, 1450-1674, 1454-1696, 1463-1681, 1465-1701, 1473-1693, |
| | 1489-1697, 1499-1701, 1500-1691, 1501-1699, 1515-1703, 1518-1703, 1537-1701, 1591-1680 |
| 27/3569792CB1/3202 | 1-583, 1-3137, 160-215, 161-215, 162-215, 175-215, 216-318, 217-318, 1077-1202, 1091-1202, 1092-1202, 1536-1767, |
| | 1536-1818, 1536-1872, 1536-1884, 1536-1910, 1536-1912, 1614-2514, 1615-2292, 1619-1866, 1619-2136, |
| | 1878-2639, 1898-2501, 1911-2458, 1911-2501, 1929-2501, 2149-2717, 2229-2673, 2240-2420, 2240-2440, 2240-2550, |
| | 2240-2557, 2240-2682, 2240-2709, 2240-2736, 2240-2766, 2240-2773, 2240-2818, 2240-2859, 2240-2866, |
| | 2240-2867, 2240-2872, 2240-2885, 2240-2887, 2240-2899, 2240-2902, 2240-2907, 2240-2922, 2240-2936, 2240-2938, |
| | 2240-2946, 2240-2991, 2240-2996, 2240-3060, 2240-3076, 2240-3081, 2240-3092, 2240-3093, 2240-3096, |
| | 2240-3099, 2240-3132, 2240-3137, 2242-3056, 2242-3093, 2243-3030, 2251-3033, 2255-3050, 2258-3015, 2272-2823, |
| | 2354-2653, 2354-2979, 2374-3202, 2375-3132, 2441-2653, 2442-3066, 2465-3002, 2478-3101, 2538-3097, |
| | 2540-2968, 2545-2982, 2560-2851 |
| 28/7500100CB1/1530 | 1-95, 1-204, 1-281, 1-324, 1-336, 1-346, 1-554, 5-208, 5-278, 5-299, 7-69, 7-285, 10-257, 20-299, 21-286, 28-554, |
| | 37-375, 42-113, 42-759, 42-779, 42-782, 42-786, 42-826, 42-827, 42-863, 42-936, 42-973, 42-992, 44-401, 46-596, |
| | 50-470, 58-511, 66-422, 104-436, 121-345, 159-453, 160-409, 165-472, 166-518, 171-422, 224-438, 227-508, 228-480, |
| | 245-490, 245-513, 258-458, 267-511, 272-561, 278-520, 281-540, 281-559, 285-530, 287-533, 288-535, 294-451, |
| | 296-515, 304-572, 340-663, 355-527, 371-504, 418-554, 437-654, 566-1349, 572-861, 649-741, 658-1441, 680-1474, |
| | 692-1446, 705-1321, 714-1471, 728-1477, |
| | 739-1479, 754-1430, 758-1019, 758-1422, 762-1445, 762-1474, 765-1475, 769-1446, 770-1057, 774-1472, 781-1074, |
| | 786-1210, 791-1414, 791-1479, 800-1446, 801-1443, 807-1038, 809-1457, 811-1021, 813-1051, 817-1034, |
| | 824-1468, 825-1073, 826-1033, 826-1470, 827-1449, 831-1321, 831-1468, 833-1476, 835-1082, 836-1463, 841-1472, |
| | 842-1462, 846-1045, 848-1114, 849-1435, 850-1449, 851-1518, 855-1120, 855-1454, 855-1470, 856-1321, |
| | 860-1454, 861-1460, 862-1454, 867-1457, 867-1475, 868-1099, 868-1110, 868-1125, 869-1472, 871-1472, 880-1490, |
| | 881-1119, 886-1514, 891-1062, 891-1193, 894-1467, 901-1163, 903-1350, |
| | 907-1518, 910-1167, 922-1160, 927-1200, 930-1199, 930-1472, 934-1187, 934-1454, 954-1518, 958-1474, 960-1482, |
| | 963-1260, 964-1300, 967-1230, 983-1213, 983-1291, 983-1457, 983-1483, 991-1223, 1000-1263, 1000-1474, |
| | 1004-1111, 1005-1356, 1005-1446, 1007-1457, 1010-1304, 1012-1243, 1012-1269, 1013-1231, 1013-1266, 1014-1258, |
| | 1014-1266, 1015-1476, 1016-1253, 1017-1225, 1018-1230, 1018-1283, 1025-1339, 1026-1258, 1027-1526, |
| | 1036-1281, 1039-1278, 1040-1304, 1042-1293, 1045-1187, 1045-1278, 1045-1529, 1048-1282, 1055-1313, 1055-1317, |
| | 1055-1530, 1058-1251, 1066-1517, 1071-1295, 1071-1347, 1075-1284, 1075-1289, 1078-1338, 1080-1354, |
| | 1083-1367, 1085-1328, 1087-1294, 1087-1304, 1087-1328, 1092-1516, 1102-1351, 1102-1354, 1102-1467, 1107-1461, |
| | 1125-1347, 1133-1374, |
| | 1133-1379, 1135-1482, 1136-1353, 1140-1415, 1171-1407, 1176-1518, 1177-1399, 1181-1400, 1193-1470, 1199-1460, |
| | 1201-1445, 1206-1516, 1211-1421, 1215-1518, 1226-1484, 1226-1529, 1229-1456, 1229-1459, 1229-1528, |
| | 1230-1502, 1231-1444, 1231-1530, 1243-1453, 1243-1483, 1248-1530, 1259-1481, 1260-1469, 1260-1530, 1269-1529, |
| | 1272-1510, 1272-1530, 1273-1488, 1279-1518, 1281-1526, 1284-1508, 1284-1525, 1287-1490, 1306-1431, |
| | 1308-1504, 1308-1530, 1309-1530, 1315-1530, 1320-1530, 1322-1501, 1325-1516, 1332-1527, 1334-1421, 1334-1513, |
| | 1334-1530, 1335-1529, 1338-1530, 1341-1530, 1343-1530, 1348-1530, 1351-1530, 1356-1530, 1358-1530, |
| | 1359-1529, 1368-1530, 1371-1478, 1381-1530, 1412-1530, 1420-1530, 1431-1520, 1441-1530, 1448-1530, 1449-1530, |
| | 1450-1530, 1467-1530 |
| 29/5201851CB1/5894 | 1-1032, 1-2167, 246-894, 364-773, 389-849, 406-1149, 427-927, 434-1041, 456-916, 493-609, 493-746, 592-845, |
| | 715-969, 716-1366, 717-1064, 775-990, 817-1381, 873-1366, 962-1303, 962-1377, 978-1552, 1034-1136, 1034-1220, |
| | 1034-1273, 1034-1304, 1034-1385, 1034-1405, 1034-1453, 1034-1469, 1034-1473, 1034-1551, 1034-1616, |
| | 1034-1645, 1034-1646, 1034-2167, 1050-1192, 1062-1540, 1071-1341, 1071-1463, 1071-1474, 1071-1521, 1071-1537, |
| | 1071-1540, 1071-1588, 1071-1618, 1071-1619, 1071-1645, 1071-1646, 1071-1647, 1071-1648, 1096-1711, |
| | 1107-1279, 1126-1472, 1149-1563, 1152-1348, 1154-1917, 1187-1698, 1187-1779, 1189-1777, 1214-1805, 1241-1528, |
| | 1251-1882, 1274-1792, 1300-1720, 1315-1596, 1330-1610, 1354-2059, 1370-1855, 1382-1645, 1401-2153, |
| | 1403-1899, 1424-2113, |
| | 1433-2168, 1438-2217, 1461-2025, 1461-2132, 1505-1718, 1505-2080, 1508-2167, 1512-2167, 1535-1821, 1549-1701, |
| | 1604-2167, 1604-2189, 1634-1968, 1654-2167, 1655-1752, 1716-2224, 1724-2167, 1737-1988, 1790-2370, |
| | 1802-2379, 1809-2271, 1819-1909, 1822-2536, 1838-2435, 1851-2015, 1851-2043, 1857-2407, 1920-2157, 1989-2283, |
| | 1999-2167, 2000-2162, 2001-2167, 2032-2538, 2037-2665, 2050-2548, 2052-2676, 2055-2595, 2068-2534, |
| | 2070-2167, 2080-2167, 2081-2364, 2085-2788, 2125-2733, 2125-2744, 2127-2166, 2127-2167, 2144-2658, 2151-2705, |
| | 2185-2712, 2192-2816, 2194-2816, |
| | 2204-2613, 2210-2816, 2223-2745, 2270-2744, 2282-2814, 2299-2666, 2300-2666, 2304-2427, 2307-2811, 2308-2991, |
| | 2339-2766, 2352-2937, 2369-2628, 2384-2891, 2385-2883, 2419-2654, 2431-2868, 2434-2829, 2438-2797, |
| | 2438-2816, 2438-2866, 2438-2868, 2439-2977, 2442-2868, 2446-2683, 2458-2676, 2461-2868, 2464-2744, 2464-2834, |
| | 2466-2598, 2466-3018, 2487-2994, 2526-3158, 2531-3095, 2534-3261, 2542-2868, 2556-2805, 2563-3080, |
| | 2566-3064, 2574-3137, 2613-3141, 2618-2932, 2626-3336, 2629-2886, 2648-3190, 2648-3277, 2669-3211, 2681-3353, |
| | 2684-3307, 2693-3322, 2701-3348, 2701-3353, 2705-2736, 2705-3354, 2715-3298, 2722-2948, 2750-3030, |
| | 2762-3254, 2766-3311, 2771-3028, 2771-3407, 2774-3503, 2778-3068, 2778-3565, 2794-3095, 2815-3104, 2827-3382, |
| | 2829-3352, 2856-2959, 2865-3431, 2923-3635, 2936-3631, 2940-3617, 2956-3205, 2959-3551, 2961-3537, |
| | 2968-3544, 2970-3163, 2993-3586, 3026-3815, 3050-3335, 3054-3586, 3062-3474, 3075-3140, 3076-3184, |
| | 3089-3380, 3090-3760, 3091-3200, 3091-3565, 3098-3802, 3105-3586, 3106-3589, 3108-3347, 3115-3738, 3124-3588, |
| | 3125-3355, 3131-3547, 3134-3399, 3139-3787, 3162-3586, 3168-3564, 3169-3350, 3175-3805, 3183-3831, |
| | 3183-3862, 3187-3656, 3189-3328, 3233-3867, 3266-3580, 3271-3818, 3271-3870, 3280-3694, 3289-3720, 3324-3836, |
| | 3330-3565, 3348-3571, 3372-3666, 3396-3868, 3398-3645, 3398-4048, 3407-3867, 3407-3882, 3411-3855, |
| | 3414-3877, 3416-3862, 3430-3867, 3449-3734, 3449-3867, 3456-3870, 3470-3867, 3473-3782, 3474-3710, 3474-3711, |
| | 3478-3513, 3478-3867, 3482-3814, 3484-3752, 3484-3795, 3484-3796, 3484-3797, 3484-3805, 3484-3813, |
| | 3484-3821, 3484-3828, 3484-3829, 3484-3830, 3484-3867, 3484-3931, 3485-3828, 3492-3867, 3498-3878, 3501-3726, |
| | 3517-3867, 3543-3789, 3560-3867, 3570-3839, 3579-4202, 3588-3883, 3594-3878, 3611-3867, 3613-3867, |
| | 3622-3831, 3642-3916, 3653-4307, 3661-4241, 3669-3892, 3690-3792, 3690-3816, 3690-3824, 3690-3825, |
| | 3690-3882, 3697-4112, 3704-3946, 3720-3885, 3726-3885, 3743-3835, 3765-4514, 3808-4175, 3811-4408, 3816-4071, |
| | 3824-4125, 3828-4077, 3840-3878, 3855-4154, 3855-4345, 3856-4097, 3875-4121, 3875-4473, 3880-4080, |
| | 3880-4103, 3883-4530, 3886-4321, 3888-4508, 3907-4156, 3907-4160, 3909-4491, 3942-4188, 3963-4674, 3968-4232, |
| | 3983-4227, 3994-4378, 4015-4594, 4057-4502, 4058-4306, 4058-4319, 4073-4266, 4160-4674, 4316-4591, |
| | 4397-4751, 4408-5051, 4408-5084, 4409-4995, 4413-5259, 4421-5004, 4426-5259, 4465-5050, 4465-5055, 4465-5070, |
| | 4465-5096, 4465-5805, 4487-4802, 4490-4986, 4495-5259, 4497-5259, 4504-4779, 4504-5118, 4510-4771, |
| | 4512-5256, 4537-5259, 4544-4831, 4548-5259, 4551-5287, 4563-5259, 4575-5256, 4576-5157, 4585-4866, 4585-4892, |
| | 4588-4889, 4589-4889, 4590-5121, 4593-4857, 4600-5259, 4601-5256, 4602-5259, 4612-4888, 4612-4890, |
| | 4640-5259, 4646-4909, 4646-5279, 4653-4988, 4653-5445, 4657-5051, 4657-5259, 4658-4946, 4662-5259, |
| | 4662-5504, 4665-5256, 4676-5118, 4688-5301, 4693-5160, 4703-5130, 4703-5340, 4708-5028, 4713-4992, 4714-5031, |
| | 4714-5057, 4755-5048, 4756-5038, 4764-5031, 4773-5065, 4796-5459, 4797-5070, 4807-5511, 4808-5093, |
| | 4821-5106, 4838-5257, 4848-5357, 4850-5357, 4855-5437, 4878-5190, 4882-5350, 4909-5367, 4920-5345, 4940-5357, |
| | 4953-5242, 4953-5607, 4969-5278, 4969-5403, 4986-5552, 4988-5566, 4999-5277, 5028-5600, 5036-5721, |
| | 5038-5756, 5048-5741, 5049-5383, 5078-5349, 5114-5402, 5114-5809, 5126-5459, 5127-5810, 5149-5477, 5155-5746, |
| | 5164-5765, 5192-5806, 5203-5753, 5206-5806, 5216-5826, 5227-5715, 5241-5622, 5241-5806, 5242-5821, |
| | 5265-5746, 5275-5894, 5291-5811, 5296-5601, 5300-5595, 5303-5624, 5305-5826, 5308-5888, 5322-5795, 5330-5799, |
| | 5352-5712, 5370-5652, 5380-5822, 5383-5826, 5391-5743, 5397-5815, 5407-5819, 5408-5819, 5416-5819, |
| | 5423-5741, 5503-5789, 5503-5831, 5508-5822, 5520-5826 |
| 30/7500667CB1/2031 | 1-250, 1-260, 1-269, 1-2014, 5-264, 5-266, 6-256, 8-257, 11-252, 29-272, 96-544, 269-919, 272-562, 286-879, 304-724, |
| | 304-885, 335-950, 341-615, 343-615, 352-634, 358-606, 369-599, 377-1029, 379-595, 381-668, 394-1009, 399-788, |
| | 417-1071, 419-1092, 445-925, 447-861, 447-862, 447-927, 447-929, 452-813, 475-1097, 479-1121, 493-1115, |
| | 515-962, 526-1194, 547-1108, 548-995, 549-1134, 581-1238, 587-1198, 589-1178, 599-1184, 602-1032, 626-908, |
| | 638-752, 641-1067, 642-956, 644-891, 644-1335, 672-1195, 678-948, 687-935, 687-948, 687-980, 687-1193, 688-965, |
| | 688-990, 697-956, 701-938, 701-1272, 708-1087, 713-971, 714-1294, 714-1467, 714-1507, 730-1199, 730-1460, |
| | 733-1223, 758-848, 769-997, 773-1069, 777-1329, 778-1028, 790-1103, 792-1181, 800-1093, 802-941, 802-1234, |
| | 804-1078, 812-1009, 813-1343, 815-1015, 821-1427, 821-1484, 822-1282, 826-958, 826-1409, 828-1071, 829-1393, |
| | 831-1122, 843-1070, 847-1181, 855-1458, 856-1453, 859-1144, 860-1524, 862-1116, 862-1131, 864-1418, |
| | 875-1163, |
| | 879-1423, 880-1448, 881-1133, 899-1485, 902-1479, 903-1173, 903-1180, 904-1533, 905-1366, 915-1216, 918-1543, |
| | 920-1181, 924-1189, 931-1469, 933-1176, 933-1552, 950-1213, 955-1189, 968-1486, 972-1226, 974-1161, |
| | 982-1404, 988-1278, 988-1565, 995-1557, 999-1545, 1006-1533, 1025-1680, 1026-1653, 1028-1671, 1046-1308, |
| | 1063-1295, 1065-1279, 1071-1179, 1071-1263, 1071-1295, 1075-1325, 1081-1610, 1083-1373, 1091-1567, 1091-1680, |
| | 1097-1334, 1098-1333, 1103-1344, 1114-1407, 1120-1401, 1124-1372, 1146-1375, 1151-1343, 1151-1429, |
| | 1153-1490, 1157-1416, 1157-1794, 1161-1453, 1164-1380, 1173-1595, 1183-1759, 1184-1371, 1192-1473, 1196-1665, |
| | 1200-1616, 1209-1471, 1214-1855, 1216-1468, 1225-1874, 1229-1566, 1231-1463, 1233-1562, 1233-1781, |
| | 1235-1486, 1237-1519, 1272-1582, 1286-1942, 1294-1967, 1297-1940, 1309-1974, 1312-1546, 1312-1613, 1312-2005, |
| | 1315-1965, 1316-1622, 1317-1559, 1319-1630, 1332-1932, 1332-1983, 1333-1560, 1333-1614, 1345-1962, |
| | 1361-1980, |
| | 1366-1661, 1367-1581, 1369-1616, 1379-1660, 1381-1626, 1390-1703, 1393-1868, 1402-2006, 1405-1871, 1410-1907, |
| | 1410-2005, 1419-2014, 1420-1621, 1430-2004, 1431-1733, 1449-1704, 1449-1905, 1453-1675, 1453-1679, |
| | 1453-1684, 1454-2009, 1454-2031, 1465-1724, 1486-1768, 1492-1759, 1493-2014, 1514-2014, 1529-1972, 1529-1992, |
| | 1531-2013, 1532-1762, 1537-2009, 1537-2031, 1547-1795, 1548-1852, 1558-1803, 1562-2018, 1565-2014, |
| | 1571-2014, 1573-1805, 1582-1841, 1584-2007, 1584-2014, 1586-2013, 1589-2010, 1590-2014, 1596-2014, 1604-2013, |
| | 1612-2018, 1613-2015, 1631-2005, 1631-2018, 1632-1861, 1638-2027, 1645-1867, 1667-1878, 1671-2011, |
| | 1679-1929, 1680-1947, 1687-1906, 1689-2027, 1697-1901, 1698-2005, 1700-2016, 1702-1936, 1707-1958, 1710-1938, |
| | 1712-1958, 1718-1942, 1719-2013, 1724-2031, 1725-2013, 1726-1854, 1742-2013, 1751-1971, 1759-1973, |
| | 1782-1933, 1833-2024, 1848-2031 |
| 31/7744055CB1/1617 | 1-641, 45-344, 49-548, 59-171, 59-632, 59-818, 78-400, 81-168, 81-619, 81-719, 83-292, 83-402, 83-403, 83-425, |
| | 83-684, 85411, 85-462, 86-545, 86-639, 88-230, 88-353, 89-342, 89-346, 89-519, 89-549, 89-656, 89-698, 90-338, |
| | 90-576, 91-201, 96-355, 97-350, 99-700, 99-740, 101-352, 101-719, 104-594, 107-788, 116-424, 121410, 128-458, |
| | 224-432, 256-453, 415-672, 415-961, 457-1000, 479-1095, 535-776, 584-1187, 586-799, 600-1283, 616-1184, 653-897, |
| | 712-1617, 744-996, 806-1065, 890-1120, 906-1130 |
| 32/7502082CB1/5758 | 1-1032, 246-894, 364-773, 389-849, 406-1149, 427-927, 434-1041, 456-916, 493-609, 493-746, 592-845, 715-969, |
| | 716-1366, 717-1064, 775-990, 817-1381, 873-1366, 962-1303, 962-1377, 978-1552, 1034-1120, 1034-1133, 1034-1136, |
| | 1034-1190, 1034-1220, 1034-1225, 1034-1232, 1034-1242, 1034-1273, 1034-1304, 1034-1354, 1034-1385, |
| | 1034-1402, 1034-1405, 1034-1426, 1034-1439, 1034-1551, 1034-1575, 1034-1616, 1034-1645, 1034-1646, 1038-5758, |
| | 1050-1192, 1050-1426, 1062-1540, 1071-1341, 1071-1463, 1071-1474, 1071-1521, 1071-1537, 1071-1540, |
| | 1071-1588, 1071-1618, 1071-1619, 1071-1645, 1071-1646, 1071-1647, 1071-1648, 1083-1426, 1096-1711, 1107-1279, |
| | 1126-1472, 1149-1563, 1152-1348, 1154-1917, 1166-1426, 1184-1426, 1187-1698, 1187-1779, 1189-1777, |
| | 1214-1805, 1224-1426, 1233-1426, 1235-1426, 1238-1426, 1241-1528, 1251-1882, 1267-1426, 1274-1792, 1283-1426, |
| | 1287-1426, |
| | 1300-1720, 1315-1596, 1316-1426, 1320-1426, 1330-1610, 1354-2059, 1370-1855, 1382-1645, 1401-2100, 1403-1899, |
| | 1424-2113, 1438-2167, 1461-2025, 1461-2074, 1505-2074, 1508-2197, 1512-2181, 1604-2154, 1604-2167, |
| | 1654-2258, 1716-2167, 1724-2258, 1802-2167, 1809-2167, 1851-2043, 1857-2167, 1920-2157, 1999-2253, 2001-2257, |
| | 2068-2345, 2080-2185, 2127-2457, 2127-2462, 2153-2467, 2186-2325, 2327-2442, 2344-2871, 2351-2975, |
| | 2353-2975, 2363-2772, 2369-2975, 2382-2904, 2429-2903, 2438-2973, 2458-2825, 2466-2970, 2467-2969, 2498-2925, |
| | 2511-3096, 2543-3050, 2544-3042, 2573-3296, 2598-3136, 2625-3177, 2646-3153, 2685-3317, 2690-3254, |
| | 2693-3420, 2722-3239, 2772-3300, 2785-3495, 2807-3287, 2807-3436, 2828-3370, 2840-3512, 2843-3466, 2852-3481, |
| | 2860-3507, 2860-3512, 2864-3430, 2874-3457, 2921-3413, 2925-3470, 2933-3662, 2937-3551, 2988-3511, |
| | 3003-3541, 3082-3732, 3095-3730, 3099-3717, 3118-3710, 3120-3696, 3127-3703, 3152-3732, 3185-3974, 3213-3732, |
| | 3257-3961, 3264-3732, 3265-3748, 3274-3897, 3298-3946, 3334-3964, 3346-3778, 3432-3977, 3432-4029, |
| | 3441-3990, 3448-3879, 3482-3741, 3483-3995, 3555-4027, 3557-4207, 3566-4026, 3570-4014, 3589-4026, 3738-4361, |
| | 3812-4466, 3820-4400, 3924-4673, 3970-4567, 4034-4632, 4042-4689, 4045-4480, 4047-4667, 4068-4650, |
| | 4122-4833, 4174-4753, 4216-4661, 4299-4924, 4450-4996, 4463-5092, 4479-5076, 4492-5120, 4507-5129, 4526-5208, |
| | 4567-5210, 4567-5243, 4568-5154, 4580-5131, 4580-5163, 4585-5418, 4586-5415, 4624-5209, 4624-5214, |
| | 4624-5229, 4624-5255, 4624-5418, 4660-5418, 4662-5418, 4663-5277, 4664-5415, 4671-5415, 4674-5418, 4706-5418, |
| | 4735-5316, 4735-5418, 4758-5418, 4759-5418, 4760-5418, 4761-5418, 4815-5414, 4821-5418, 4821-5663, |
| | 4824-5415, 4852-5319, 4866-5447, 4880-5418, 5068-5526, 5128-5562, 5147-5758 |
| 33/7502084CB1/5292 | 1-1032, 246-894, 364-773, 389-849, 406-1149, 427-927, 434-1041, 456-916, 493-609, 493-746, 592-845, 715-969, |
| | 716-1366, 717-1064, 775-990, 817-1381, 873-1366, 962-1303, 962-1377, 978-1552, 1034-1120, 1034-1133, 1034-1136, |
| | 1034-1190, 1034-1220, 1034-1225, 1034-1232, 1034-1242, 1034-1273, 1034-1304, 1034-1354, 1034-1385, |
| | 1034-1402, 1034-1405, 1034-1426, 1034-1439, 1034-1551, 1034-1575, 1034-1616, 1034-1645, 1034-1646, 1050-1192, |
| | 1050-1426, 1062-1540, 1071-1341, 1071-1463, 1071-1474, 1071-1521, 1071-1537, 1071-1540, 1071-1588, |
| | 1071-1618, 1071-1619, 1071-1645, 1071-1646, 1071-1647, 1071-1648, 1083-1426, 1096-1711, 1107-1279, 1126-1472, |
| | 1149-1563, 1152-1348, 1154-1917, 1166-1426, 1184-1426, 1187-1698, 1187-1779, 1189-1777, 1214-1805, |
| | 1224-1426, 1233-1426, 1235-1426, 1238-1426, 1241-1528, 1251-1882, 1267-1426, 1274-1792, 1283-1426, 1287-1426, |
| | 1300-1720, 1315-1596, 1316-1426, 1320-1426, 1330-1610, 1354-2059, 1370-1855, 1382-1645, 1401-2100, |
| | 1403-1899, 1424-2113, 1438-2167, 1461-2025, 1461-2074, 1505-2074, 1508-2197, 1512-2181, 1604-2154, 1604-2167, |
| | 1654-2258, 1716-2167, 1724-2258, 1802-2167, 1809-2167, 1851-2043, 1857-2167, 1920-2157, 1999-2253, |
| | 2001-2257, 2068-2345, 2080-2185, 2127-2457, 2127-2462, 2153-2467, 2186-2325, 2327-2442, 2327-2523, 2327-2529, |
| | 2327-2594, 2327-2693, 2327-2695, 2327-2697, 2327-2707, 2327-2754, 2327-2815, 2327-2817, 2327-2824, |
| | 2327-2835, 2327-2864, 2327-2903, 2344-2871, 2351-2975, 2353-2975, 2363-2772, 2369-2975, 2382-2904, 2429-2903, |
| | 2438-2973, 2458-2825, 2466-2970, 2467-2969, 2498-2925, 2511-3096, 2543-3050, 2544-3042, 2573-3296, |
| | 2598-3136, 2625-3177, 2646-3153, 2685-3317, 2690-3254, 2693-3420, 2722-3239, 2772-3300, 2785-3495, 2807-3287, |
| | 2807-3436, 2828-3370, 2840-3512, 2843-3466, 2852-3481, 2860-3507, 2860-3512, 2864-3430, 2874-3457, |
| | 2921-3413, 2925-3470, 2933-3662, 2988-3511, 3003-3541, 3082-3732, 3095-3730, 3099-3728, 3118-3710, |
| | 3120-3696, 3127-3703, 3152-3737, 3185-3848, 3213-3737, 3234-3299, 3257-3835, 3264-3737, 3265-3732, 3274-3771, |
| | 3283-3732, 3290-3706, 3298-3820, 3321-3827, 3334-3838, 3342-3732, 3346-3732, 3411-3660, 3424-3899, |
| | 3425-3705, 3425-3732, 3430-3851, 3430-3903, 3443-3944, 3448-3753, 3461-3915, 3483-3869, 3487-3715, 3489-3724, |
| | 3507-3730, 3514-3813, 3555-3901, 3557-4081, 3560-4038, 3566-3900, 3570-3888, 3589-3900, 3622-3915, |
| | 3634-3868, 3643-3691, 3729-4145, 3730-3925, 3733-4235, 3733-4274, 3733-4340, 3737-3979, 3758-3839, 3798-4547, |
| | 3844-4441, 3908-4506, 3916-4563, 3919-4354, 3921-4541, 3942-4524, 3996-4707, 4048-4627, 4090-4535, |
| | 4400-5082, 4441-5084, 4441-5117, 4442-5028, 4454-5005, 4454-5037, 4498-5083, 4498-5088, 4498-5103, 4498-5129, |
| | 4530-5292, 4536-5292, 4537-5151, 4545-5289, 4545-5292, 4620-5292, 4635-5288, 4635-5292, 4661-5288, |
| | 4686-5292, 4689-5288, 4728-5292, 4762-5292, 4775-5292, 4839-5292 |
| 34/7502085CB1/5549 | 1-1032, 246-894, 364-773, 389-849, 406-1149, 427-927, 434-1041, 456-916, 493-609, 493-746, 592-845, 715-969, |
| | 716-1366, 717-1064, 775-990, 775-1033, 817-1381, 873-1366, 962-1303, 962-1377, 978-1552, 1034-1225, 1034-1474, |
| | 1034-1540, 1034-1551, 1034-1588, 1034-1616, 1034-1618, 1034-1619, 1034-1645, 1034-1646, 1034-1647, |
| | 1034-1648, 1050-1192, 1050-1426, 1063-1537, 1063-1647, 1096-1711, 1107-1279, 1129-1472, 1149-1563, 1152-1348, |
| | 1154-1917, 1166-1426, 1187-1698, 1187-1779, 1189-1777, 1214-1805, 1241-1528, 1251-1882, 1274-1792, |
| | 1300-1720, 1315-1596, 1316-1426, 1330-1610, 1357-2059, 1370-1855, 1382-1645, 1401-2100, 1403-1899, 1424-2113, |
| | 1438-2311, 1461-2025, 1461-2074, 1505-1717, 1505-2074, 1508-2167, 1512-2167, 1604-2154, 1604-2178, |
| | 1654-2167, 1716-2224, |
| | 1724-2167, 1790-2370, 1802-2379, 1822-2536, 1838-2435, 1851-2043, 1857-2521, 1920-2157, 1999-2167, 2001-2167, |
| | 2032-2538, 2037-2665, 2050-2548, 2052-2676, 2055-2595, 2068-2167, 2068-2534, 2080-2167, 2085-2788, |
| | 2125-2733, 2125-2744, 2144-2658, 2151-2705, 2152-2814, 2168-2298, 2168-2303, 2185-2712, 2192-2816, 2194-2816, |
| | 2204-2613, 2210-2816, 2223-2745, 2270-2744, 2299-2666, 2307-2811, 2308-2810, 2339-2766, 2352-2937, |
| | 2384-2891, 2385-2883, 2414-3137, 2439-2977, 2466-3018, 2487-2994, 2526-3158, 2531-3095, 2534-3261, 2563-3080, |
| | 2613-3141, 2626-3336, 2648-3128, |
| | 2648-3277, 2669-3211, 2681-3353, 2684-3307, 2693-3322, 2701-3348, 2701-3353, 2705-3271, 2715-3298, 2762-3254, |
| | 2766-3311, 2774-3503, 2829-3352, 2844-3382, 2865-3431, 2923-3573, 2936-3571, 2940-3569, 2959-3551, |
| | 2961-3537, 2968-3544, 2993-3578, 3026-3689, 3054-3578, 3062-3474, 3075-3140, 3098-3676, 3105-3578, 3106-3573, |
| | 3115-3612, 3124-3586, 3131-3547, 3139-3661, 3162-3668, 3175-3679, 3187-3573, 3252-3501, 3266-3546, |
| | 3401-3879, 3475-3709, 3484-3532, 3570-3986, 3587-3766, 3587-3820, 3587-4076, 3587-4115, 3587-4181, 3639-4388, |
| | 3685-4282, 3723-4143, 3749-4347, 3757-4404, 3760-4195, 3762-4382, 3783-4365, 3837-4548, 3889-4468, |
| | 3931-4376, 3976-4413, 3976-4415, 3976-4455, 3976-4534, 3976-4543, 3976-4549, 3976-4610, 3976-4635, 3976-4672, |
| | 3976-4691, 4014-4639, 4425-5161, 4448-5133, 4493-5111, 4555-4987, 4678-5161, 4688-5311, 4868-5386, |
| | 4889-5381, 4912-5549, 4998-5549 |
| 35/7502093CB1/4741 | 1-540, 84-571, 84-720, 122-652, 145-818, 145-842, 149-672, 171-1435, 189-669, 189-741, 189-913, 222-915, 224-912, |
| | 228-608, 228-804, 229-913, 231-807, 235-608, 235-788, 235-885, 235-886, 235-912, 235-913, 235-914, 245-730, |
| | 261-914, 275-487, 284-883, 300-509, 301-855, 317-693, 329-807, 363-978, 393-739, 416-830, 419-615, 421-1184, |
| | 433-693, 454-965, 454-1046, 456-1044, 481-1072, 508-795, 518-1149, 541-1059, 567-987, 582-863, 597-877, |
| | 624-1326, 637-1122, 649-912, 668-1367, 670-1166, 691-1380, 705-1578, 728-1292, 728-1341, 772-1341, 775-1434, |
| | 779-1434, 802-1088, 816-968, 871-1421, 871-1445, 901-1235, 922-1233, 983-1491, 1004-1255, 1057-1637, 1069-1646, |
| | 1076-1538, 1086-1176, 1089-1803, 1105-1702, 1118-1282, 1118-1310, 1124-1788, 1245-1548, 1256-1550, |
| | 1299-1805, 1304-1932, 1317-1815, |
| | 1319-1943, 1322-1862, 1335-1801, 1348-1631, 1352-2055, 1360-1539, 1392-2000, 1392-2011, 1411-1925, 1418-1972, |
| | 1419-2081, 1435-1565, 1435-1570, 1452-1979, 1459-2083, 1461-2083, 1471-1880, 1477-2083, 1490-2012, |
| | 1537-2011, 1566-1933, 1574-2078, 1575-2077, 1606-2033, 1619-2204, 1651-2158, 1652-2150, 1681-2404, 1706-2244, |
| | 1733-2285, 1754-2261, 1784-2049, 1793-2425, 1798-2362, 1801-2528, 1830-2347, 1880-2408, 1893-2603, |
| | 1915-2395, 1915-2544, 1936-2478, 1948-2620, 1951-2574, 1960-2589, 1968-2615, 1968-2620, 1972-2538, 1982-2565, |
| | 2029-2521, 2033-2578, 2041-2770, 2096-2619, 2111-2649, 2132-2698, 2190-2840, 2203-2838, 2207-2836, |
| | 2226-2818, 2228-2804, 2235-2811, 2260-2845, 2293-2956, 2321-2845, 2329-2741, 2342-2407, 2365-2943, 2372-2845, |
| | 2373-2840, 2391-2853, 2398-2814, 2406-2928, 2429-2935, 2442-2946, 2454-2840, 2519-2768, 2532-3007, |
| | 2533-2813, 2533-2840, 2551-3052, 2556-2861, 2569-3023, 2595-2823, 2597-2832, 2615-2838, 2622-2921, 2668-3146, |
| | 2730-3023, 2742-2976, 2751-2799, 2837-3253, 2854-3033, 2854-3087, 2854-3343, 2854-3382, 2854-3448, |
| | 2906-3655, 2952-3549, 2981-3019, 2990-3410, 3016-3614, 3024-3671, 3027-3462, 3029-3649, 3050-3632, 3104-3815, |
| | 3156-3735, 3198-3643, 3508-4190, 3549-4192, 3549-4225, 3550-4136, 3562-4145, 3567-4400, 3606-4191, |
| | 3606-4196, 3606-4211, 3606-4237, 3631-4127, 3638-4400, 3645-4259, 3653-4397, 3666-4400, 3678-4400, 3687-4400, |
| | 3688-4400, 3689-4400, 3704-4400, 3716-4397, 3717-4298, 3731-4262, 3741-4400, 3742-4397, 3743-4400, |
| | 3781-4400, 3787-4420, 3794-4586, 3797-4396, 3803-4400, 3803-4645, 3806-4397, 3829-4442, 3834-4301, 3848-4429, |
| | 3937-4600, 3948-4652, 3996-4578, 4050-4508, 4094-4741, 4110-4544, 4127-4693, 4129-4707, 4169-4741 |
| 36/7502097CB1/4900 | 1-540, 84-571, 84-720, 122-652, 145-818, 145-842, 149-672, 228-804, 229-741, 238-608, 238-788, 238-913, 240-608, |
| | 245-730, 245-807, 245-885, 245-912, 245-913, 261-886, 261-914, 267-693, 273-693, 275-487, 282-693, 284-883, |
| | 300-509, 301-644, 301-648, 301-819, 301-855, 317-459, 317-693, 329-807, 350-693, 363-978, 374-546, 393-739, |
| | 416-830, 419-615, 421-1184, 433-693, 451-693, 454-965, 454-1046, 456-1044, 481-1072, 491-693, 502-693, |
| | 505-693, 508-795, 518-1149, 534-693, 541-1059, 554-693, 567-987, 582-863, 597-877, 624-1326, 637-1122, 649-912, |
| | 668-1367, 670-1166, 691-1380, 705-1434, 728-1292, 728-1341, 772-984, 772-1341, 775-1464, 779-1434, 871-1421, |
| | 871-1434, 921-1525, 983-1434, 991-1525, 1057-1434, 1069-1434, 1118-1310, 1124-1434, 1187-1424, 1266-1520, |
| | 1268-1524, 1335-1611, |
| | 1347-1452, 1394-1724, 1394-1729, 1453-1592, 1594-1861, 1594-1960, 1594-1962, 1594-1964, 1594-1974, 1594-2021, |
| | 1594-2084, 1594-2091, 1594-2102, 1594-2131, 1594-2159, 1594-2170, 1594-2214, 1611-2138, 1618-2242, |
| | 1620-2242, 1630-2039, 1636-2242, 1649-2171, 1660-2240, 1696-2170, 1725-2092, 1733-2237, 1734-2236, 1765-2192, |
| | 1778-2363, 1810-2317, 1811-2309, 1840-2563, 1865-2403, 1892-2444, 1913-2420, 1952-2584, 1957-2521, |
| | 1960-2687, 1989-2506, 2039-2567, 2052-2762, 2074-2554, 2074-2703, 2095-2637, 2107-2779, 2110-2733, 2119-2748, |
| | 2127-2774, 2127-2779, 2131-2697, 2141-2724, 2188-2680, 2192-2737, 2200-2929, 2255-2778, 2270-2808, |
| | 2291-2857, 2349-2999, 2362-2997, 2366-2995, 2385-2977, 2387-2963, 2394-2970, 2419-3004, 2452-3115, 2480-3004, |
| | 2488-2900, 2501-2566, 2524-3102, 2531-3004, 2532-2999, 2557-2973, 2565-3087, 2588-3094, 2601-3105, |
| | 2613-2999, |
| | 2678-2927, 2691-3166, 2692-2972, 2692-2999, 2710-3211, 2715-3020, 2728-3182, 2754-2982, 2756-2991, 2774-2997, |
| | 2781-3080, 2827-3305, 2889-3182, 2901-3135, 2910-2958, 2996-3412, 3013-3192, 3013-3246, 3013-3502, |
| | 3013-3541, 3013-3607, 3065-3814, 3111-3708, 3140-3178, 3149-3569, 3175-3773, 3183-3830, 3186-3621, 3188-3808, |
| | 3209-3791, 3263-3974, 3315-3894, 3357-3802, 3667-4349, 3708-4351, 3708-4384, 3709-4295, 3721-4304, |
| | 3726-4559, 3765-4350, 3765-4355, 3765-4370, 3765-4396, 3790-4286, 3797-4559, 3804-4418, 3812-4556, 3825-4559, |
| | 3837-4559, 3846-4559, 3847-4559, 3848-4559, 3863-4559, 3875-4556, 3876-4457, 3890-4421, 3900-4559, |
| | 3901-4556, 3902-4559, 3940-4559, 3946-4579, 3953-4745, 3956-4555, 3962-4559, 3962-4804, 3965-4556, 3988-4601, |
| | 3993-4460, 4007-4588, 4096-4759, 4107-4811, 4155-4737, 4209-4667, 4253-4900, 4269-4703, 4286-4852, |
| | 4288-4866, 4328-4900 |
| 37/7502108CB1/4942 | 1-540, 84-571, 84-720, 122-652, 145-818, 145-842, 149-672, 231-807, 235-913, 236-913, 236-914, 267-693, 273-693, |
| | 275-487, 282-693, 284-883, 301-509, 301-608, 301-644, 301-648, 301-741, 301-788, 301-819, 301-855, 301-885, |
| | 301-886, 301-912, 301-913, 301-915, 317-459, 317-693, 329-730, 329-807, 330-608, 330-804, 330-914, 350-693, |
| | 363-978, 374-546, 393-739, 416-830, 419-615, 421-1184, 433-693, 451-693, 454-965, 454-1046, 456-1044, |
| | 481-1072, 491-693, 500-693, 502-693, 505-693, 508-795, 518-1149, 534-693, 541-1059, 550-693, 554-693, 567-987, |
| | 582-863, 583-693, 597-877, 624-1326, 637-1122, 649-912, 668-1367, 670-1166, 691-1380, 705-1578, 728-1292, |
| | 728-1341, 772-984, 772-1341, 775-1434, 779-1434, |
| | 871-1421, 871-1445, 921-1434, 983-1491, 991-1434, 1057-1637, 1069-1646, 1089-1803, 1105-1702, 1118-1310, |
| | 1124-1788, 1187-1424, 1266-1434, 1268-1434, 1299-1805, 1304-1932, 1317-1815, 1319-1943, 1322-1862, 1335-1434, |
| | 1335-1801, 1347-1434, 1352-2055, 1392-2000, 1392-2011, 1394-1433, 1394-1434, 1411-1925, 1418-1972, |
| | 1419-2081, 1452-1979, 1459-2083, 1461-2083, 1471-1880, 1477-2083, 1490-2012, 1537-2011, 1566-1933, 1574-2078, |
| | 1575-2077, 1606-2033, 1619-2204, 1651-2158, 1652-2150, 1681-2404, 1706-2244, 1733-2285, 1754-2261, |
| | 1793-2425, 1798-2362, 1801-2528, 1830-2347, 1880-2408, 1893-2603, 1915-2395, 1915-2544, 1936-2478, 1948-2620, |
| | 1951-2574, 1960-2589, 1968-2615, 1968-2620, 1972-2538, 1982-2565, 2029-2521, 2033-2578, 2041-2770, |
| | 2096-2619, |
| | 2111-2649, 2132-2698, 2190-2840, 2203-2838, 2207-2836, 2226-2818, 2228-2804, 2235-2811, 2260-2845, 2293-2853, |
| | 2321-2845, 2329-2741, 2342-2407, 2365-2853, 2372-2845, 2373-2840, 2382-3005, 2391-2855, 2398-2814, |
| | 2406-2853, 2429-2853, 2442-2853, 2454-2840, 2519-2768, 2532-2853, 2533-2813, 2533-2840, 2551-2853, 2556-2853, |
| | 2569-2853, 2595-2823, 2597-2832, 2615-2838, 2622-2853, 2668-2853, 2742-2842, 2751-2799, 2846-3469, |
| | 2920-3574, 2928-3508, 2936-3159, 2971-3213, 3032-3781, 3078-3675, 3116-3536, 3142-3740, 3150-3797, 3153-3588, |
| | 3155-3775, 3176-3758, 3230-3941, 3282-3861, 3324-3769, 3369-3806, 3369-3808, 3369-3848, 3369-3927, |
| | 3369-3936, 3369-3942, 3369-4003, 3369-4028, 3369-4065, 3369-4084, 3407-4032, 3818-4554, 3841-4526, 3886-4504, |
| | 3948-4380, 4071-4554, 4081-4704, 4261-4779, 4282-4774, 4305-4942, 4391-4942 |
| 38/7500668CB1/2144 | 1-333, 11-320, 14-253, 18-336, 19-281, 19-322, 22-325, 23-269, 23-326, 25-2144, 33-271, 34-278, 34-292, 36-305, |
| | 37-286, 41-300, 41-302, 41-316, 41-318, 44-275, 44-293, 46-243, 46-296, 48-266, 97-332, 200-660, 327-573, 327-584, |
| | 327-585, 327-593, 327-613, 327-853, 327-904, 336-609, 345-798, 378-678, 379-1035, 402-995, 420-840, 420-1001, |
| | 438-904, 451-1066, 457-731, 459-731, 468-750, 474-722, 485-715, 493-1145, 495-711, 497-784, 510-1125, |
| | 515-904, 533-1187, 535-1208, 545-816, 561-814, 561-1041, 563-906, 563-977, 563-978, 563-1043, 563-1045, 564-842, |
| | 564-906, 566-1008, 568-929, 569-929, 591-1213, 593-849, 595-1237, 609-1231, 631-1078, 639-861, 642-1310, |
| | 649-946, 659-965, 663-1224, 664-1111, 665-1250, 670-925, 682-864, 682-918, 688-911, 688-976, 690-929, 697-1354, |
| | 703-1314, 705-1294, 706-960, 715-1300, 718-1148, 742-1024, 754-868, 754-964, 757-1183, 758-1072, 760-1007, |
| | 760-1451, |
| | 764-974, 769-993, 788-1311, 790-976, 794-1064, 803-1051, 803-1064, 803-1096, 803-1309, 804-1081, 804-1106, |
| | 813-1072, 817-1054, 817-1388, 824-1203, 829-1087, 830-1410, 830-1583, 830-1623, 844-1386, 846-1315, 846-1386, |
| | 846-1576, 849-1339, 874-964, 885-1113, 889-1185, 893-1445, 894-1144, 906-1219, 908-1297, 916-1209, 918-1057, |
| | 918-1350, 920-1194, 928-1125, 929-1459, 931-1131, 937-1543, 937-1600, 938-1398, 942-1074, 942-1525, |
| | 944-1187, 945-1509, 947-1238, 959-1186, 963-1297, 971-1574, 972-1569, 975-1260, 976-1640, 978-1232, 978-1247, |
| | 980-1534, 991-1279, 995-1539, 996-1564, 997-1249, 1015-1601, 1018-1595, 1019-1289, 1019-1296, 1020-1649, |
| | 1021-1482, 1031-1332, 1034-1659, 1036-1297, 1040-1305, 1047-1585, 1049-1292, 1049-1668, 1066-1329, |
| | 1071-1305, 1084-1602, 1088-1342, 1090-1277, 1098-1520, 1104-1394, 1104-1681, 1111-1673, 1115-1661, 1122-1649, |
| | 1141-1796, 1142-1769, |
| | 1144-1787, 1162-1424, 1179-1411, 1181-1395, 1187-1295, 1187-1379, 1187-1411, 1187-2111, 1191-1441, 1197-1726, |
| | 1199-1489, 1207-1683, 1207-1796, 1213-1450, 1214-1449, 1219-1460, 1226-1501, 1230-1523, 1236-1517, |
| | 1240-1488, 1262-1491, 1267-1459, 1267-1545, 1269-1606, 1273-1532, 1273-1910, 1277-1569, 1280-1496, 1289-1711, |
| | 1299-1875, 1300-1487, 1308-1533, 1308-1589, 1312-1781, 1316-1732, 1325-1550, 1325-1587, 1328-1638, |
| | 1330-1971, 1332-1584, 1341-1990, 1342-1601, 1345-1682, 1347-1579, 1349-1678, 1349-1897, 1351-1602, 1353-1614, |
| | 1353-1635, 1372-1647, 1375-1642, 1386-2043, 1388-1698, 1402-2058, 1407-1659, 1410-2083, 1411-1654, |
| | 1413-2056, 1425-2090, 1428-1651, 1428-1662, 1428-1729, 1428-2121, 1431-2081, 1432-1738, 1433-1675, 1435-1746, |
| | 1448-2048, 1448-2099, 1449-1676, 1449-1730, 1461-2078, 1477-2096, 1482-1777, 1483-1697, 1485-1732, |
| | 1495-1776, 1497-1742, 1506-1819, 1509-1984, 1518-2122, 1521-1987, 1526-2023, 1526-2121, 1535-2130, |
| | 1536-1737, 1546-2120, 1547-1849, 1565-1820, 1565-2021, 1569-1791, 1569-1795, 1569-1800, 1570-2125, 1570-2144, |
| | 1581-1840, 1602-1884, 1608-1875, 1609-2130, 1622-2130, 1630-2130, 1645-2088, 1645-2108, 1647-2129, |
| | 1648-1878, 1653-2125, 1653-2144, 1663-1911, 1664-1968, 1674-1919, 1678-2134, 1681-2130, 1687-2130, 1689-1921, |
| | 1698-1957, 1700-2123, 1700-2130, 1702-2129, 1705-2126, 1706-2130, 1712-2130, 1720-2129, 1728-2134, |
| | 1729-2131, 1747-2121, 1747-2134, 1748-1977, 1754-2143, 1761-1983, 1783-1994, 1787-2127, 1795-2045, 1796-2063, |
| | 1803-2022, 1805-2143, 1809-2130, 1813-2017, 1814-2121, 1816-2132, 1818-2052, 1823-2074, 1826-2054, |
| | 1828-2074, 1834-2058, 1835-2129, 1840-2144, 1841-2129, 1842-1970, 1858-2129, 1867-2087, 1875-2089, 1898-2049, |
| | 1899-2115, 1899-2123, 1903-2144, 1912-2130, 1914-2144, 1922-2142, 1938-2144, 1948-2144, 1949-2140, |
| | 1950-2144, 1964-2144, 1967-2138, 1986-2144, 2040-2129 |
| 39/7505114CB1/1216 | 1-256, 1-576, 1-1216, 22-301, 22-337, 22-390, 22-428, 22-436, 22-441, 22-442, 22-452, 22-518, 22-519, 22-575, 22-598, |
| | 22-612, 22-615, 22-620, 22-631, 22-635, 22-650, 22-651, 67-224, 376-576, 441-823, 453-897, 462-1161, 467-1079, |
| | 507-1161, 518-1161, 558-913, 561-1161, 565-1161, 610-1161, 638-750, 638-1115, 660-1161, 667-1161, 730-1161, |
| | 741-1161, 750-1138, 769-1161, 774-1161, 777-1161, 887-1161, 892-1209 |
| 40/7506452CB1/818 | 1-273, 1-277, 15-818, 105-324, 109-350, 111-351, 111-356, 111-369, 111-745, 116-342, 116-349, 116-350, 116-357, |
| | 119-336, 119-370, 119-392, 123-770, 124-324, 128-347, 131-350, 133-355, 134-338, 137-457, 213-386, 467-805, |
| | 467-818, 482-737, 516-818, 599-790, 709-818, 712-810 |
| 41/7506730CB1/833 | 1-463, 1-833, 54-293, 101-748, 101-751, 103-259, 138-403, 138-463, 461-719, 513-778 |
| 42/7505046CB1/1505 | 1-264, 1-415, 1-426, 1-520, 1-522, 1-543, 1-1190, 1-1207, 2-419, 22-362, 25-302, 28-497, 46-531, 46-533, 83-398, |
| | 83-572, 129-432, 133-452, 173-449, 200-405, 296-543, 538-1128, 554-1084, 564-1172, 569-817, 569-1060, 569-1068, |
| | 569-1115, 569-1124, 569-1185, 574-1113, 574-1154, 579-987, 579-1151, 587-1089, 587-1090, 591-1119, 596-1161, |
| | 599-1174, 601-870, 616-1120, 616-1183, 621-1190, 632-825, 643-944, 656-900, 657-1190, 666-1129, 669-1222, |
| | 671-965, 674-968, 680-933, 698-1126, 702-1188, 702-1206, 710-1185, 714-1190, 725-1019, 726-1505, 727-1038, |
| | 730-1186, 731-1134, 734-1132, 741-1198, 742-1191, 745-994, 746-1192, 747-1184, 752-997, 754-1072, 758-1184, |
| | 763-1191, 770-1186, 773-1071, 775-1101, 775-1186, 776-1210, 777-1040, 779-1082, 789-1191, 804-1015, |
| | 804-1121, 804-1154, 804-1190, 835-1039, 867-1193, 872-1081, 882-1190, 891-1114, 905-1149, 912-1132, 922-1182, |
| | 941-1191, 979-1173, 979-1232, 1113-1249 |
| 43/7506453CB1/889 | 1-858, 7-265, 7-266, 22-206, 25-264, 26-130, 98-261, 99-191, 101-265, 103-181, 103-243, 103-259, 104-239, 108-201, |
| | 114-244, 115-808, 116-228, 264-431, 264-463, 264-470, 264-471, 264-473, 264-474, 264-490, 264-500, 264-501, |
| | 264-510, 264-706, 264-710, 264-773, 264-782, 264-801, 264-805, 264-809, 264-826, 264-832, 264-834, 264-845, |
| | 264-855, 264-861, 264-862, 265-514, 265-535, 265-559, 266-832, 267-487, 267-542, 268-724, 268-838, 271-504, |
| | 271-786, 271-798, 271-839, 271-859, 271-863, 273-535, 274-754, 276-863, 277-871, 278-440, 280-861, 282-861, |
| | 282-863, 283-535, 283-870, 288-837, 288-857, 288-862, |
| | 290-366, 291-764, 291-802, 292-862, 293-531, 295-789, 296-539, 297-478, 297-813, 297-882, 298-537, 300-552, |
| | 300-839, 300-853, 300-862, 301-868, 303-554, 304-813, 305-508, 305-543, 305-822, 305-848, 305-855, 306-558, |
| | 306-871, 307-871, 309-843, 313-555, 316-565, 316-811, 318-541, 318-546, 318-553, 318-566, 318-587, 318-596, |
| | 319-855, 321-804, 323-728, 323-863, 325-566, 325-889, 326-839, 326-862, 327-551, 327-855, 327-863, 327-864, |
| | 328-617, 329-822, 329-845, 329-857, 330-848, 332-585, 332-596, 339-646, 342-868, 343-587, 344-549, 344-627, |
| | 345-611, 345-626, 346-816, 347-792, 349-579, 349-583, |
| | 349-589, 349-595, 349-864, 351-592, 351-629, 351-861, 354-856, 356-604, 356-616, 356-814, 359-551, 359-597, |
| | 359-605, 360-627, 360-855, 360-864, 361-578, 362-614, 362-643, 364-554, 365-563, 365-596, 365-601, 366-608, |
| | 366-632, 368-600, 370-614, 371-612, 371-655, 371-780, 373-614, 374-601, 374-621, 374-636, 374-822, 375-599, |
| | 376-656, 376-657, 376-754, 376-839, 377-625, 377-839, 378-634, 379-808, 379-846, 382-838, 383-625, 383-645, |
| | 383-678, 385-652, 385-653, 386-861, 387-861, 388-610, 388-613, 388-652, 393-592, 393-610, 393-863, 395-589, |
| | 395-855, 399-859, 403-837, 404-653, 404-666, 406-769, 406-863, 409-849, 409-862, 412-856, 412-864, 413-598, |
| | 415-683, 415-701, 415-863, 416-670, 418-864, 420-652, 420-841, 421-665, 421-862, 421-864, 422-670, 423-637, |
| | 423-864, 424-864, 425-847, 425-863, 425-864, 425-889, 426-661, 426-862, 426-864, 431-863, 433-811, 435-661, |
| | 442-837, 442-841, 443-697, |
| | 446-717, 448-681, 449-635, 451-822, 453-747, 453-839, 454-673, 454-704, 457-650, 458-707, 458-864, 459-692, |
| | 459-696, 459-863, 462-676, 463-652, 463-683, 465-681, 465-701, 466-682, 467-848, 472-695, 472-855, 477-864, |
| | 481-749, 485-863, 486-862, 487-741, 487-871, 488-713, 488-752, 489-839, 491-618, 491-863, 496-752, 502-833, |
| | 502-864, 504-857, 509-848, 512-749, 512-841, 515-862, 520-775, 521-763, 523-785, 527-778, 531-776, 534-734, |
| | 534-744, 534-750, 539-780, 549-778, 549-806, 550-813, 551-815, 551-824, 553-866, 554-863, 557-824, 558-815, |
| | 558-862, 570-825, 570-850, 572-754, 574-814, 584-759, 591-810, 592-863, 593-810, 595-823, 596-862, 599-828, |
| | 602-785, 607-863, 608-863, 620-848, 626-848, 627-863, 628-820, 630-863, 635-855, 635-858, 635-863, 636-863, |
| | 637-828, 637-839, 646-863, 647-863, 650-862, 654-863, 657-863, 668-863, 670-841, 677-841, 684-860, 692-863, |
| | 714-837, 718-849, 720-863, 724-863, 727-863, 731-863, 743-845, 746-862, 747-863, 750-848, 750-863, 773-859 |
| 44/7509967CB1/1066 | 1-249, 1-1061, 5-390, 7-265, 9-332, 10-390, 22-174, 22-206, 25-174, 26-130, 27-375, 34-305, 41-301, 58-420, 95-342, |
| | 97-316, 97-329, 97-330, 97-331, 97-332, 97-338, 97-348, 97-352, 97-363, 97-371, 98-325, 99-191, 99-306, 99-314, |
| | 99-328, 99-339, 99-345, 99-346, 99-356, 100-343, 101-236, 101-282, 101-283, 101-289, 101-296, 101-297, 101-300, |
| | 101-306, 101-314, 101-318, 101-323, 101-325, 101-327, 101-330, 101-331, 101-332, 101-334, 101-336, 101-339, |
| | 101-340, 101-341, 101-342, 101-343, 101-345, 101-346, 101-347, 101-348, 101-349, 101-350, 101-351, 101-354, |
| | 101-355, 101-357, 101-358, 101-359, 101-362, 101-363, 101-373, 101-375, 102-245, 102-302, 102-317, 102-323, |
| | 102-325, 102-330, 102-332, 102-333, 102-335, 102-340, 102-342, 102-343, 102-344, 102-346, 102-347, 102-348, |
| | 102-349, 102-351, 102-355, 102-363, 102-382, 102-384, 103-179, 103-288, 103-289, 103-298, 103-299, 103-304, |
| | 103-308, 103-313, 103-315, 103-317, 103-320, 103-321, 103-322, 103-323, 103-325, 103-329, 103-330, 103-331, |
| | 103-332, 103-333, 103-334, 103-335, 103-336, 103-337, 103-339, 103-340, 103-342, 103-343, 103-344, 103- |
| | 104-308, 104-313, 104-327, 104-328, 104-336, 104-339, 104-343, 104-345, 104-346, 104-349, 104-352, 104-354, |
| | 104-356, 104-357, 104-368, 105-297, 105-302, 105-310, 105-316, 105-317, 105-328, 105-331, 105-334, 105-335, |
| | 105-337, 105-340, 105-344, 105-346, 105-348, 105-349, 105-350, 105-351, 105-353, 105-355, 105-356, 105-357, |
| | 105-358, 105-359, 105-360, 105-362, 105-366, 105-380, 105-381, 106-300, 106-319, 106-333, 106-337, 106-340, |
| | 106-346, 106-347, 106-349, 106-353, 106-355, 106-360, 106-363, 106-365, 106-367, 106-369, 107-346, 108-238, |
| | 108-283, 108-290, 108-301, 108-306, 108-311, 108-313, 108-316, 108-322, 108-324, 108-327, 108-328, 108-329, |
| | 108-332, 108-333, 108-334, 108-335, 108-336, 108-337, 108-338, 108-340, 108-341, 108-342, 108-345, 108-347, |
| | 108-348, 108-349, 108-350, 108-351, 108-352, 108-353, 108-354, 108-355, 108-356, 108-358, 108-359, 108-360, |
| | 108-361, 108-363, 108-364, |
| | 108-365, 108-367, 108-368, 108-369, 108-370, 108-373, 108-376, 108-377, 108-379, 108-381, 108-383, 108-403, |
| | 109-323, 109-338, 109-349, 109-357, 109-359, 109-360, 110-312, 110-331, 110-332, 110-362, 110-375, 111-292, |
| | 111-297, 111-302, 111-303, 111-305, 111-312, 111-315, 111-316, 111-324, 111-325, 111-326, 111-327, 111-328, |
| | 111-330, 111-331, 111-332, 111-333, 111-335, 111-336, 111-337, 111-338, 111-339, 111-340, 111-342, 111-343, |
| | 111-344, 111-345, 111-346, 111-347, 111-348, 111-350, 111-351, 111-352, 111-353, 111-354, 111-355, 111-356, |
| | 111-357, 111-358, 111-359, 111-360, 111-361, 111-362, 111-363, 111-364, 111-367, 111-368, 111-369, 111-370, |
| | 111-371, 111-374, 111-375, 111-380, 111-387, 111-390, 111-392, 111-756, 111-791, 111-871, 113-313, 113-325, |
| | 113-328, 113-330, 113-338, 113-341, 113-342, 113-351, 113-352, 113-353, 113-361, 113-363, 113-365, 113-366, |
| | 113-393, 114-315, 114-342, |
| | 114-343, 114-349, 114-350, 114-355, 114-362, 114-367, 115-327, 115-330, 115-335, 115-339, 115-342, 115-350, |
| | 115-352, 115-355, 115-361, 115-364, 115-367, 115-378, 116-278, 116-299, 116-301, 116-309, 116-310, 116-316, |
| | 116-320, 116-328, 116-329, 116-331, 116-334, 116-337, 116-340, 116-342, 116-345, 116-347, 116-349, 116-350, |
| | 116-351, 116-353, 116-355, 116-356, 116-357, 116-359, 116-360, 116-361, 116-367, 116-371, 116-373, 116-385, |
| | 116-409, 117-359, 117-360, 117-871, 118-310, 118-317, 118-345, 118-346, 118-348, 118-356, 118-357, 118-360, |
| | 118-367, 119-343, 119-361, 119-367, 119-871, 120-314, 120-339, 120-364, 120-369, 121-359, 121-362, 121-364, |
| | 121-374, 121-404, 122-336, 122-355, 122-356, 122-359, 122-365, 122-366, 122-367, 122-368, 122-369, 122-396, |
| | 122-406, 123-335, 123-342, 123-382, 125-288, 125-315, 125-321, 125-326, 125-334, 125-338, 125-339, 125-340, |
| | 125-345, 125-346, 125-347, |
| | 125-349, 125-352, 125-353, 125-354, 125-356, 125-358, 125-360, 125-361, 125-362, 125-366, 125-367, 125-368, |
| | 125-369, 125-370, 125-373, 125-374, 125-375, 125-376, 125-385, 125-386, 125-387, 125-391, 125-392, 125-411, |
| | 125-417, 126-308, 126-330, 129-358, 129-449, 130-350, 130-361, 130-365, 130-376, 131-375, 131-385, 132-323, |
| | 132-346, 132-359, 132-362, 132-372, 132-373, 132-393, 133-332, 133-336, 133-351, 133-359, 133-366, 133-378, |
| | 133-379, 135-366, 136-345, 136-350, 136-357, 136-365, 136-379, 140-362, 140-363, 140-364, 140-379, 140-390, |
| | 141-327, 141-346, 141-348, 141-364, 142-324, 142-335, 142-338, 142-356, 142-362, 142-366, 142-369, 142-370, |
| | 142-383, 142-387, 143-368, 148-356, 148-373, 155-409, 156-377, 156-383, 156-386, 173-443, 175-407, 175-410, |
| | 175-448, 178-437, 197-435, 326-587, 329-577, 358-429, 712-1051, 730-948, 756-1066, 823-1051, 921-1052, 950-1066 |

TABLE 5

| Polynucleotide SEQ | | |
|---|---|---|
| ID NO: | Incyte Project ID: | Representative Library |
| 23 | 7497502CB1 | SINTNOR01 |
| 24 | 7103532CB1 | TONSDIT01 |
| 25 | 7500108CB1 | PITUDIR01 |
| 26 | 7500665CB1 | ADRETUT05 |
| 27 | 3569792CB1 | HNT2UNN03 |
| 28 | 7500100CB1 | ADRETUT05 |
| 29 | 5201851CB1 | ADMEDRV02 |
| 30 | 7500667CB1 | ADRETUT05 |
| 31 | 7744055CB1 | ADRETUT05 |
| 32 | 7502082CB1 | PLACFEB01 |
| 33 | 7502084CB1 | PLACFEB01 |
| 34 | 7502085CB1 | PLACFEB01 |
| 35 | 7502093CB1 | PLACFEB01 |
| 36 | 7502097CB1 | PLACFEB01 |
| 37 | 7502108CB1 | PLACFEB01 |
| 38 | 7500668CB1 | ADRETUT07 |
| 39 | 7505114CB1 | LIVRDIR01 |
| 40 | 7506452CB1 | PITUNOT01 |
| 41 | 7506730CB1 | UTRSNOT02 |
| 42 | 7505046CB1 | SCORNON02 |
| 43 | 7506453CB1 | PITUNOT03 |
| 44 | 7509967CB1 | PITUNOT01 |

TABLE 6

| Library | Vector | Library Description |
|---|---|---|
| ADMEDRV02 | PCR2-TOPOTA | Library was constructed using pooled cDNA from different donors. |
| | | cDNA was generated using mRNA isolated from the |
| | | following: aorta, para-aortic soft tissue, fetal femur, untreated |
| | | epidermal keratinocytes, neck muscle, supraglottic soft |
| | | tissue, calf muscle, retroperitoneal soft tissue, sacral bone |
| | | giant cell tumor, treated breast skin fibroblast cells, abdominal |
| | | skin, untreated T-lymphocyte cell line (Jurkat cell line), fetal small |
| | | intestine, fetal colon, colon tumor (grade 3 colonic |
| | | adenocarcinoma) small intestine, colon, ascending colon, diseased |
| | | descending colon tissue (chronic ulcerative colitis, |
| | | moderate to severe), cecal tumor (grade 1 neuroendocrine carcinoma), |
| | | diseased ileum tissue (Crohn's disease), diseased |
| | | small intestine (focal reactive foveolar hyperplasia consistent with |
| | | bile reflux), ascending colon, fetal stomach, diseased |
| | | gallbladder (moderate chronic cholecystitis and cholelithiasis), esophagus, |
| | | diseased gallbladder (acute hemorrhagic cholecystitis with cholelithiasis), |
| | | esophagus tumor (invasive grade 3 |
| | | adenocarcinoma), stomach, diseased gallbladder (chronic cholecystitis |
| | | and cholelithiasis), diseased gallbladder (acute |
| | | necrotizing cholecystitis with cholelithiasis (clinically hydrops), |
| | | endometrium, diseased cervix tissue (mild chronic |
| | | cervicitis with focal squamous metaplasia), uterus tumor (leiomyoma), |
| | | diseased ovary (polycystic ovarian disease), |
| | | myometrium, uterus, endometrial tumor (grade 3 adenosquamous |
| | | carcinoma) ovary, fetal penis, testis, untreated prostate |
| | | epithelial cells (PrEC Cells), testicle tumor (embryonal carcinoma), |
| | | seminal vesicle, diseased prostate (adenofibromatous |
| | | hyperplasia), fetal spleen, spleen, thymus, diseased tonsil tissue |
| | | (reactive lymphoid hyperplasia). from diseased spleen |
| | | (idiopathic thrombocytopenic purpura), spleen tumor |
| | | (malignant lymphoma, diffuse large |
| | | cell type, B-cell phenotype with abundant reactive T-cells), thymus, |
| | | diseased tonsil tissue (lymphoid hyperplasia), pelvic |
| | | lymph node (matched with Hodgkin's disease, nodular sclerosing type), |
| | | a treated chronic myelogenous leukemia precursor |
| | | cell line (K562 Cells), axillary lymph node tumor (metastatic |
| | | adenocarcinoma), fetal liver, fetal pancreas, pancreas, liver |
| | | tumor (metastatic grade 2 (of 4) neuroendocrine carcinoma), |
| | | fetal kidney, renal pyramid, kidney tumor (renal cell |
| | | carcinoma, clear cell type), diseased kidney tissue (chronic |
| | | interstitial nephritis), ureter tumor (transitional cell carcinoma), |
| | | kidney cortex, ureter tumor (invasive grade 3 (of 3) transitional cell |
| | | carcinoma), pooled lung, adrenal gland, benign parotid |
| | | tumor (sebaceous lymphadenoma), parotid, thyroid, diseased |
| | | thyroid (adenomatous hyperplasia), diseased breast |
| | | (proliferative fibrocystic changes), breast, submandibular gland, |
| | | adrenal tumor (pheochromocytoma), and hyperplastic parathyroid. |
| ADRETUT05 | pINCY | Library was constructed using RNA isolated from |
| | | adrenal tumor tissue removed from a 52-year-old Caucasian female |
| | | during a unilateral adrenalectomy. Pathology |
| | | indicated a pheochromocytoma. |
| ADRETUT07 | pINCY | Library was constructed using RNA isolated from adrenal |
| | | tumor tissue removed from a 43-year-old Caucasian female |
| | | during a unilateral adrenalectomy. Pathology indicated |
| | | pheochromocytoma. |
| HNT2UNN03 | PSPORT1 | This normalized NT2 cell line library was constructed from |
| | | independent clones from an untreated NT2 cell line library. |
| | | Starting RNA was made from the NT2 cell line derived from a |
| | | human teratocarcinoma, which exhibited properties |
| | | characteristic of a committed neuronal precursor at an early |
| | | stage of development. The cells were untreated. The library |
| | | was normalized in two rounds using conditions adapted from |
| | | Soares et al., PNAS (1994) 91: 9228-9232 and Bonaldo et al., |
| | | Genome Research 6 (1996): 791, except that a significantly |
| | | longer (48 hours/round) reannealing hybridization was used. |
| LIVRDIR01 | pINCY | The library was constructed using RNA isolated from diseased |
| | | liver tissue removed from a 63-year-old Caucasian female |
| | | during a liver transplant. Patient history included primary biliary |
| | | cirrhosis diagnosed in 1989. Serology was positive for |
| | | anti-mitochondrial antibody. |
| PITUDIR01 | PCDNA2.1 | This random primed library was constructed using RNA |
| | | isolated from pituitary gland tissue removed from a 70-year-old |
| | | female who died from metastatic adenocarcinoma. |
| PITUNOT01 | PBLUESCRIPT | Library was constructed using RNA obtained from Clontech (CLON |
| | | 6584-2, lot 35278). The RNA was isolated from the |
| | | pituitary glands removed from a pool of 18 male and female |
| | | Caucasian donors, 16 to 70 years old, who died from trauma. |
| PITUNOT03 | PSPORT1 | Library was constructed using RNA isolated from pituitary tissue |
| | | of a 46-year-old Caucasian male, who died from colon |
| | | cancer. Serologies were negative. Patient history included arthritis, |
| | | peptic ulcer disease, and tobacco use. Patient |
| | | medications included Tagamet and muscle relaxants. |
| PLACFEB01 | pINCY | Library was constructed using pooled cDNA from two different donors. cDNA |
| | | was generated using RNA isolated from |
| | | placenta tissue removed from a Caucasian fetus (donor A), who |
| | | died after 16 weeks' gestation from fetal demise and |
| | | hydrocephalus; and a Caucasian male fetus (donor B), who died |
| | | after 18 weeks' gestation from fetal demise. Patient history |
| | | included umbilical cord wrapped around the head (3 times) and the |
| | | shoulders (1 time) in donor A. Serology was positive |
| | | for anti-CMV in donor A. Family history included multiple pregnancies |
| | | and live births, and an abortion in donor A. |
| SCORNON02 | PSPORT1 | This normalized spinal cord library was constructed from 3.24M |
| | | independent clones from the a spinal cord tissue library. |
| | | RNA was isolated from the spinal cord tissue removed from a |
| | | 71-year-old Caucasian male who died from respiratory |
| | | arrest. Patient history included myocardial infarction, gangrene, and end |
| | | stage renal disease. The normalization and |
| | | hybridization conditions were adapted from Soares et |
| | | al.(PNAS (1994) 91: 9228). |
| SINTNOR01 | PCDNA2.1 | This random primed library was constructed using RNA isolated |
| | | from small intestine tissue removed from a 31-year-old |
| | | Caucasian female during Roux-en-Y gastric bypass. Patient |
| | | history included clinical obesity. |
| TONSDIT01 | pINCY | Library was constructed using RNA isolated from the tonsil tissue |
| | | of a 6-year-old Caucasian male during |
| | | adenotonsillectomy. Pathology indicated lymphoid hyperplasia |
| | | of the tonsils. The patient presented with an abscess of the |
| | | pharynx. The patient was not taking any medications. Family history |
| | | included hypothyroidism in the grandparent(s) and |
| | | benign skin neoplasm in the sibling(s). |
| UTRSNOT02 | PSPORT1 | Library was constructed using RNA isolated from uterine tissue |
| | | removed from a 34-year-old Caucasian female during a |
| | | vaginal hysterectomy. Patient history included mitral valve disorder. |
| | | Family history included stomach cancer, congenital |
| | | heart anomaly, irritable bowel syndrome, ulcerative colitis, colon |
| | | cancer, cerebrovascular disease, type II diabetes, and depression. |

TABLE 7

| Program | Description | Reference | Parameter Threshold |
|---|---|---|---|
| ABI | A program that removes vector sequences and | Applied Biosystems, Foster City, CA. | |
| FACTURA | masks ambiguous bases in nucleic acid sequences. | | |
| ABI/PARACEL | A Fast Data Finder useful in comparing and | Applied Biosystems, Foster City, CA; | Mismatch <50% |
| FDF | annotating amino acid or nucleic acid sequences. | Paracel Inc., Pasadena, CA. | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability |
| | sequence similarity search for amino acid and | 215: 403-410; Altschul, S. F. et al. (1997) | value = 1.0E−8 or |
| | nucleic acid sequences. BLAST includes five | Nucleic Acids Res. 25: 3389-3402. | less Full Length |
| | functions: blastp, blastn, blastx, tblastn, and tblastx. | | sequences: Probability |
| | | | value = 1.0E−10 or |
| | | | less |
| FASTA | A Pearson and Lipman algorithm that searches for | Pearson, W. R. and D. J. Lipman (1988) Proc. | ESTs: fasta E |
| | similarity between a query sequence and a group of | Natl. Acad Sci. USA 85: 2444-2448; | value = 1.06E−6 |
| | sequences of the same type. FASTA comprises as | Pearson, W. R. (1990) Methods | Assembled ESTs: fasta |
| | least five functions: fasta, tfasta, fastx, tfastx, and | Enzymol. 183: 63-98; and Smith, | Identity = 95% or |
| | ssearch. | T. F. and M. S. Waterman (1981) Adv. | greater and Match |
| | | Appl. Math. 2: 482-489. | length = 200 bases |
| | | | or greater; fastx E |
| | | | value = 1.0E−8 or less |
| | | | Full Length sequences: |
| | | | fastx score = |
| | | | 100 or greater |
| BLIMPS | A BLocks IMProved Searcher that matches a | Henikoff, S. and J. G. Henikoff (1991) | Probability value = |
| | sequence against those in BLOCKS, PRINTS, | Henikoff (1991) Nucleic Acids Res. 19: | 1.0E−3 or less |
| | DOMO, PRODOM, and PFAM databases to search | 6565-6572; Henikoff, J. G. and S. | |
| | for gene families, sequence homology, and | Henikoff (1996) Methods Enzymol. 266: | |
| | structural fingerprint regions. | 88-105; and Attwood, T. K. et al. (1997) J. | |
| | | Chem. Inf. Comput. Sci. 37: 417-424. | |
| HMMER | An algorithm for searching a query sequence | Krogh, A. et al. (1994) J. Mol. Biol. | PFAM, INCY, |
| | against hidden Markov model (HMM)-based | 235: 1501-1531; Sonnhammer, E. L. L. et al. | SMART, or TIGRFAM |
| | databases of protein family consensus sequences, | (1988) Nucleic Acids Res. 26: 320-322; | hits: Probability |
| | such as PFAM, INCY, SMART, and TIGRFAM. | Durbin, R. et al. (1998) Our World View, in a | value = 1.0E−3 |
| | | Nutshell, Cambridge Univ. Press, pp. 1-350. | or less Signal peptide |
| | | | hits: Score = |
| | | | 0 or greater |
| ProfileScan | An algorithm that searches for structural and sequence | Gribskov, M. et al. (1988) CABIOS 4: 61-66; | Normalized quality |
| | motifs in protein sequences that match sequence patterns | Gribskov, M. et al. (1989) Methods Enzymol. | score ≧ GCG- |
| | defined in Prosite. | 183: 146-159; Bairoch, A. et al. (1997) | specified “HIGH” |
| | | Nucleic Acids Res. 25: 217-221. | value for that |
| | | | particular Prosite motif. |
| | | | Generally, score = |
| | | | 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = |
| | CrossMatch, programs based on efficient implementation | Appl. Math. 2: 482-489; Smith, T. F. and | 120 or greater; |
| | of the Smith-Waterman algorithm, useful in searching | M. S. Waterman (1981) J. Mol. Biol. | Match length = |
| | sequence homology and assembling DNA sequences. | 147: 195-197; and Green, P., | 56 or greater |
| | | University of Washington, | |
| | | Seattle, WA. | |
| Consed | A graphical tool for viewing and editing Phrap | Gordon, D. et al. (1998) Genome | |
| | assemblies. | Res. 8: 195-202. | |
| SPScan | A weight matrix analysis program that scans protein | Nielson, H. et al. (1997) Protein Engineering | Score = |
| | sequences for the presence of secretory signal peptides. | 10: 1-6; Claverie, J. M. and S. Audic (1997) | 3.5 or greater |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. | |
| | delineate transmembrane segments on protein sequences | (1998) Proc. Sixth Intl. Conf. on Intelligent | |
| | and determine orientation. | Systems for Mol. Biol., Glasgow et al., eds., | |
| | | The Am. Assoc. for Artificial Intelligence | |
| | | Press, Menlo Park, CA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| | | | | | | | | | | | African | | |
|---|---|---|---|---|---|---|---|---|---|---|---|---|---|
| SEQ | | | | | | | | | | Caucasian | Allele 1 | Asian | Hispanic |
| ID | | | | EST | CB1 | EST | | | Amino | Allele 1 | fre- | Allele 1 | Allele 1 |
| NO: | PID | EST ID | SNP ID | SNP | SNP | Allele | Allele 1 | Allele 2 | Acid | frequency | quency | frequency | frequency |
| 44 | 7509967 | 096527H1 | SNP00141453 | 209 | 317 | C | C | T | I57 | n/a | n/a | n/a | n/a |
| 44 | 7509967 | 097172H1 | SNP00061933 | 199 | 425 | C | C | T | T93 | n/a | n/a | n/a | n/a |
| 44 | 7509967 | 097461H1 | SNP00123377 | 96 | 207 | C | C | G | L21 | n/d | n/d | n/d | n/d |
| 44 | 7509967 | 097473H1 | SNP00141452 | 171 | 297 | G | G | A | V51 | n/a | n/a | n/a | n/a |
| 44 | 7509967 | 098148H1 | SNP00025887 | 44 | 142 | C | C | T | noncoding | n/d | n/d | n/d | n/d |
| 44 | 7509967 | 110743R6 | SNP00025888 | 227 | 351 | C | C | T | H69 | n/a | n/a | n/a | n/a |
| 44 | 7509967 | 110743R6 | SNP00061933 | 302 | 426 | C | C | T | P94 | n/a | n/a | n/a | n/a |
| 44 | 7509967 | 110743R6 | SNP00123377 | 84 | 208 | C | C | G | P21 | n/d | n/d | n/d | n/d |
| 44 | 7509967 | 110743R6 | SNP00141452 | 174 | 298 | G | G | A | G51 | n/a | n/a | n/a | n/a |
| 44 | 7509967 | 110743R6 | SNP00141453 | 194 | 318 | C | C | T | H58 | n/a | n/a | n/a | n/a |
| 44 | 7509967 | 110743T6 | SNP00109110 | 31 | 964 | T | T | C | noncoding | n/d | n/d | n/d | n/d |
| 44 | 7509967 | 110743T6 | SNP00155283 | 235 | 760 | C | C | A | noncoding | n/a | n/a | n/a | n/a |
| 44 | 7509967 | 112721F1 | SNP00109110 | 89 | 963 | T | T | C | noncoding | n/d | n/d | n/d | n/d |
| 44 | 7509967 | 112721F1 | SNP00155283 | 293 | 759 | C | C | A | noncoding | n/a | n/a | n/a | n/a |
| 44 | 7509967 | 1756143H1 | SNP00025888 | 243 | 350 | C | C | T | F68 | n/a | n/a | n/a | n/a |
| 44 | 7509967 | 1756146H1 | SNP00061934 | 229 | 571 | C | C | T | S142 | n/d | 0.77 | 0.71 | 0.65 |
| 44 | 7509967 | 1757095H1 | SNP00155282 | 249 | 606 | C | C | G | L154 | n/a | n/a | n/a | n/a |
| 44 | 7509967 | 1758561H1 | SNP00069331 | 147 | 948 | C | C | T | noncoding | n/a | n/a | n/a | n/a |
| 44 | 7509967 | 1758561H1 | SNP00144521 | 86 | 887 | C | C | G | noncoding | n/a | n/a | n/a | n/a |
| 44 | 7509967 | 1759734H1 | SNP00134847 | 48 | 427 | C | C | T | P94 | n/a | n/a | n/a | n/a |
| 44 | 7509967 | 1759734H1 | SNP00144520 | 149 | 528 | A | A | G | T128 | n/a | n/a | n/a | n/a |
| 44 | 7509967 | 1759948R6 | SNP00025887 | 41 | 144 | C | C | T | noncoding | n/d | n/d | n/d | n/d |
| 44 | 7509967 | 1759948R6 | SNP00025888 | 252 | 352 | C | C | T | A69 | n/a | n/a | n/a | n/a |
| 44 | 7509967 | 1759948R6 | SNP00061933 | 327 | 427 | C | C | T | P94 | n/a | n/a | n/a | n/a |
| 44 | 7509967 | 1759948R6 | SNP00123377 | 109 | 209 | C | C | G | L21 | n/d | n/d | n/d | n/d |
| 44 | 7509967 | 1759948R6 | SNP00141452 | 199 | 299 | G | G | A | V51 | n/a | n/a | n/a | n/a |
| 44 | 7509967 | 1759948R6 | SNP00141453 | 219 | 319 | C | C | T | P58 | n/a | n/a | n/a | n/a |
| 44 | 7509967 | 1759948T6 | SNP00109110 | 25 | 965 | T | T | C | noncoding | n/d | n/d | n/d | n/d |
| 44 | 7509967 | 1759948T6 | SNP00155283 | 229 | 761 | C | C | A | noncoding | n/a | n/a | n/a | n/a |
| 44 | 7509967 | 1760118H1 | SNP00093224 | 221 | 723 | A | A | C | noncoding | n/a | n/a | n/a | n/a |
| 44 | 7509967 | 5914804H1 | SNP00025887 | 21 | 148 | C | C | T | T1 | n/d | n/d | n/d | n/d |
| 44 | 7509967 | 5914804H1 | SNP00123377 | 86 | 213 | C | C | G | R23 | n/d | n/d | n/d | n/d |
| 44 | 7509967 | 5914837H1 | SNP00134847 | 90 | 436 | C | C | T | T97 | n/a | n/a | n/a | n/a |
| 44 | 7509967 | 6032626H1 | SNP00025889 | 97 | 785 | A | A | G | noncoding | n/a | n/a | n/a | n/a |

1. An isolated polypeptide selected from the group consisting of:
a) a polypeptide comprising an amino acid sequence selected from the group consisting of SEQ ID NO:1-22,
b) a polypeptide comprising a naturally occurring amino acid sequence at least 90% identical to an amino acid sequence selected from the group consisting of SEQ ID NO:2-7, SEQ ID NO:9, SEQ ID NO:16, and SEQ ID NO:19-21,
c) a polypeptide comprising a naturally occurring amino acid sequence at least 99% identical to the amino acid sequence of SEQ ID NO:1,
d) a polypeptide comprising a naturally occurring amino acid sequence at least 95% identical to the amino acid sequence of SEQ ID NO:22,
e) a polypeptide consisting essentially of a naturally occurring amino acid sequence at least 90% identical to an amino acid sequence selected from the group consisting of SEQ ID NO:17-18,
f) a biologically active fragment of a polypeptide having an amino acid sequence selected from the group consisting of SEQ ID NO:1-22, and
g) an immunogenic fragment of a polypeptide having an amino acid sequence selected from the group consisting of SEQ ID NO:1-22.

2. An isolated polypeptide of claim 1 comprising an amino acid sequence selected from the group consisting of SEQ ID NO:1-22.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:23-44.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-22.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:23-44,
b) a polynucleotide comprising a naturally occurring polynucleotide sequence at least 90% identical to a polynucleotide sequence selected from the group consisting of SEQ ID NO:23-30 and SEQ ID NO:32-42,
c) a polynucleotide comprising a naturally occurring polynucleotide sequence at least 96% identical to the polynucleotide sequence of SEQ ID NO:31,
d) a polynucleotide comprising a naturally occurring polynucleotide sequence at least 94% identical to the polynucleotide sequence of SEQ ID NO:43,
e) a polynucleotide comprising a naturally occurring polynucleotide sequence at least 91% identical to the polynucleotide sequence of SEQ ID NO:44,
f) a polynucleotide complementary to a polynucleotide of a),
g) a polynucleotide complementary to a polynucleotide of b),
h) a polynucleotide complementary to a polynucleotide of c),
i) a polynucleotide complementary to a polynucleotide of d),
j) a polynucleotide complementary to a polynucleotide of e), and
k) an RNA equivalent of a)-j).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (cenceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-22.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-99. (canceled)
